(19) United States
(12) Patent Application Publication
(10) Pub. No.: US 2009/0131502 A1
(43) Pub. Date: May 21, 2009

(54) Biologically active compounds

(76) Inventors: Martin Quibell, Cambridge (GB); John Paul Watts, Cambridge (GB); Yikang Wang, Cambridge (GB); Lee Patient, Cambridge (GB); Jonathan R. Heal, Cambridge (GB)

Correspondence Address: EDWARDS ANGELL PALMER & DODGE LLP, P.O. BOX 55874, BOSTON, MA, 02205, US

(21) Appl. No.: 12/072,414

(22) Filed: Feb. 25, 2008

Related U.S. Application Data
(63) Continuation of application No. PCT/GB2006/003155, filed on Aug. 23, 2006.

(30) Foreign Application Priority Data
Aug. 23, 2005 (GB) 0517279.6

(51) Int. Cl. A61K 31/407; A61P 19/00; A61P 33/06; A61P 35/00; G01N 33/53; C07D 487/04; C07D 491/04

(52) U.S. Cl. 514/414; 548/453; 435/7.93; 514/421

(57) ABSTRACT

The present invention relates to compounds of formula (I), and pharmaceutically acceptable salts thereof. The invention further relates to pharmaceutical compositions comprising compounds of formula (I), and the use of such compounds in the treatment of a disease selected from osteoporosis, Paget's disease, Chagas's disease, malaria, gingival diseases, hypercalaemia, metabolic bone disease and diseases involving matrix or cartilate degradation.

### RELATED APPLICATIONS

This is a continuation patent application that claims priority to PCT patent application number PCT/GB2006/003155, filed on Aug. 23, 2006, which claims priority to GB patent application to 0517279.6, filed on Aug. 23, 2005, the entirety of which are herein incorporated by reference.

### FIELD OF INVENTION

The present invention relates to compounds that are inhibitors of a broad range of cystein proteinases, pharmaceutical compositions containing said compounds, and their use of therapy. More specifically, but not exclusively, the invention relates to compounds that are inhibitors of cathepsin K and related cystein proteinases of the CA clan. Such compounds are particularly useful for the in vivo therapeutic treatment of diseases in which participation of a cysteine proteinase in implicated.

### BACKGROUND TO THE INVENTION

Proteinases form a substantial group of biological molecules which to date constitute approximately 2% of all the gene products identified following analysis of several completed genome sequencing programmes. Proteinases have evolved to participate in an enormous range of biological processes, mediating their effect by cleavage of peptide amide bonds within the myriad of proteins found in nature. This hydrolytic action is performed by initially recognizing, then binding to, particular three-dimensional electronic surfaces displayed by a protein, which align the bond for cleavage precisely within the proteinase catalytic site. Catalytic hydrolysis then commences through nucleophilic attack of the amide bond to be cleaved either via an amino acid side-chain of the proteinase itself, or through the action of a water molecule that is bound to and activated by the proteinase. Proteinases in which the attacking nucleophile is the thiol side-chain of a Cys residue are known as cysteine proteinases. The general classification of ‘cysteine proteinase’ contains many members found in a wide range of organisms from viruses, bacteria, protozoa, plants and fungi to mammals.

Cathepsin K and indeed many other crucial proteinases belong to the papain-like CAC1 family. Cysteine proteinases are classified into ‘clans’ based upon a similarity in the three-dimensional structure to a conserved arrangement of catalytic residues within the proteinase primary sequence. Additionally, ‘clans’ may be further classified into ‘families’ in which each proteinase shares a statistically significant relationship with other members when comparing the portions of amino acid sequence which constitute the parts reasonable for the proteinase activity (see Barrett, A. J. et al., in ‘Handbook of Proteolytic Enzymes’, Eds. Barrett, A. J., Rawlings, N. D., and Woessner, J. F. Publ. Academic Press, 1998, for a thorough discussion).

To date, cysteine proteinases have been classified into five clans, CA, CB, CC, CD and CE (Barrett, A. J. et al, 1998). A proteinase from the tropical papaya fruit ‘papain’ forms the foundation of clan CA, which currently contains over 80 distinct and complete entries in various sequence databases, with many more expected from the current genome sequencing efforts. Proteinases of clan CA/family C1 have been implicated in a multitude of house-keeping roles and disease processes. e.g. human proteinases such as cathepsin K (osteoporosis, osteoarthritis), cathepsin S (multiple sclerosis, rheumatoid arthritis, autoimmune disorders), cathepsin L (metastases), cathepsin B (metastases, arthritis), cathepsin F (antigen processing), cathepsin V (T-cell selection), dipeptidyl peptidase I (granulocyte serine proteinase activation) or parasitic proteinases such as falcipain (malaria parasite Plasmodium falciparum) and cruzipain (Trypanosoma cruzi infection). Recently a bacterial proteinase, staphylopain (S. aureus infection) has also been tentatively assigned to clan CA.

X-ray crystallographic structures are available for a range of the above mentioned proteinases in complex with a range of inhibitors e.g. papain (PDB entries, 1pad, 1pe6, 1pip, 1pop, 4pad, 5pad, 6pad, 1ppp, 1the, 1csb, 1huc), cathepsin K (1au0, 1au2, 1au3, 1au4, 1atk, 1mem, 1bgo, 1ayw, 1ayu, 1 n16, 1n1j, 1q6k, 1snk, 1tu6), cathepsin L (1cs8, 1mhw), cathepsin S (1glo, 1ms6, 1npz), cathepsin V (1fh0), dipeptidyl peptidase I (1jqp, 1k3b), cathepsin B (1gmy, 1csb), cathepsin F (1m6d), cruzain (a recombinant form of cruzipain see Eakin, A. E. et al, 268(9), 6115-6118, 1993) (1ewp, 1aim, 2aim, 1F29, 1F2A, 1F2B, 1F2C), staphylopain (1cv8). Each of the structures displays a similar overall active-site topology, as would be expected by their ‘clan’ and ‘family’ classification and such structural similarity exemplifies one aspect of the difficulties involved in discovering a selective inhibitor of cathepsin K suitable for human use. However, subtle differences in terms of the depth and intricate shape of the active site groove of each CAC1 proteinase are evident, which may be exploited for selective inhibitor design. Additionally, many of the current substrate-based inhibitor complexes of CAC1 family proteinases show a series of conserved hydrogen bonds between the inhibitor and the proteinase backbone, which contribute significantly to inhibitor potency. Primarily a bidentate hydrogen-bond is observed between the proteinase Gly66 (C═O)/inhibitor N—H and the proteinase Gly66(NH)/inhibitor (C═O), where the inhibitor (C═O) and (NH) are provided by an amino acid residue NHCHRCO that constitutes the S2 sub-site binding element within the inhibitor (see Berger, A. and Schecter, I. Philos. Trans. R. Soc. Lond. [Biol.], 257, 249-264, 1970 for a description of proteinase binding site nomenclature). A further hydrogen-bond between the proteinase main-chain (C═O) of asparagine or aspartic acid (158 to 163, residue number varies between proteinases) and an inhibitor (N—H) is often observed, where the inhibitor (N—H) is provided by the SI sub-site binding element within the inhibitor. Thus, the motif X—NHCHRCO—NH—Y is widely observed amongst the prior art substrate-based inhibitors of CAC1 proteinases.

Cathepsin K is thought to be significant in diseases involving excessive loss of bone or cartilage. Bone consists of a protein matrix incorporating hydroxyapatite crystals. About 90% of the structural protein of the matrix is type I collagen, with the remainder comprising various non-collagenous proteins such as osteocalcin, proteoglycans, osteopontin, osteonectin, thrombospondin, fibronectin and bone sialoprotein.

Skeletal bone is not a static structure but continually undergoes a cycle of bone resorption and replacement. Bone resorption is carried out by osteoclasts, which are multinuclear cells of haematopoietic lineage. Osteoclasts adhere to the bone surface and form a tight sealing zone. The membrane on the apical surface of the osteoclasts is folded so as to create a closed extracellular compartment between the osteoclast and the bone surface, which is acidified by proton pumps in the osteoclast membrane. Proteolytic enzymes are secreted into the compartment from the osteoclast. The high acidity in the compartment causes the hydroxyapatite at the surface of the bone to be dissolved and the proteolytic enzymes break down the protein matrix causing a resorption lacuna to be formed. Following bone resorption, osteoblasts produce a new protein matrix that is subsequently mineralised.

In disease states such as osteoporosis and Paget's disease, the bone resorption and replacement cycle is disrupted leading to a net loss of bone with each cycle. This leads to weakening of the bone and therefore to increased risk of bone fracture.

Cathepsin K is expressed at a high level in osteoclasts and is therefore thought to be essential for bone resorption. Thus, selective inhibition of cathepsin K is likely to be effective in the treatment of diseases involving excessive bone loss. These include osteoporosis, gingival diseases such as gingivitis and periodontitis, Paget's disease, hypercalaemia of malignancy and metabolic bone disease.

In addition to osteoclasts, high levels of cathepsin K are also found in chondroclasts from the synovium of osteoarthritic patients. It therefore appears that cathepsin K inhibitors will be of use in the treatment of diseases involving matrix or cartilage degradation, in particular osteoarthritis and rheumatoid arthritis.

Elevated levels of cathepsin K are also found in metastatic neoplastic cells which suggests that cathepsin K inhibitors may also be useful for treating certain neoplastic diseases.

In the prior art, the development of cysteine proteinase inhibitors for human use has recently been an area of intense activity (e.g. see Bromme, D. and Kaleta, J., Curr. Pharm. Des., 8, 1639-1658, 2002; Kim, W. and Kang, K., Expert Opin Ther. Patents, 12(3), 419-432, 2002; Leung-Toung, R. et al. Curr. Med. Chem., 9, 979-1002, 2002; Lecaille, F. et al., Chem. Rev., 102, 4459-4488, 2002; Hernandez, A. A. and Roush, W. R, Curr. Opin Chem. Biol., 6, 459-465, 2002). Considering the CAC1 family members, particular emphasis has been placed upon the development of inhibitors of human cathepsins, primarily cathepsin K (osteoporosis), cathepsin S (autoimmune disorders), cathepsin L (metastases), cathepsin B (metastases, arthritis), cathepsin F (antigen processing), cathepsin V (T-ell selection) and dipeptidyl peptidase I (granulocyte serine proteinase activation), through the use of peptide and peptidomimetic nitriles (e.g. see WO-A-03041649, WO-A-03037892, WO-A-03029200, WO-A-02051983, WO-A-02020485, US-A-20020086996, WO-A-01096285, WO-A-0109910, WO-A-0051998, WO-A-0119816, WO-A-9924460, WO-A-0049008, WO-A-0048992, WO-A-0049007, WO-A-0130772, WO-A-0055125, WO-A-0055126, WO-A-0119808, WO-A-0149288, WO-A-0147886), linear and cyclic peptide and peptidomimetic ketones (e.g. see Veber, D. F. and Thompson, S. K., Curr. Opin. Drug Discovery Dev., 3(4), 362-369, 2000, WO-A-02092563, WO-A-02017924, WO-A-01095911, WO-A-0170232, WO-A-0178734, WO-A-0009653, WO-A-0069855, WO-A-0029408, WO-A-0134153 to WO-A-0134160, WO-A-0029408, WO-A-9964399, WO-A-9805336, WO-A-9850533), ketoheterocycles (e.g. see WO-A-02080920, WO-A-03042197, WO-A-WO-A-03024924, WO-A-0055144, WO-A-0055124), monobactams (e.g. see WO-A-0059881, WO-A-9948911, WO-A-0109169), α-ketoamides (e.g. see WO-A-03013518), cyanoamides (WO-A-01077073, WO-A-01068645), dihydro pyrimidines (e.g. see WO-A-02032879) and cyanoaminopyrimidines (e.g. see WO-A-03020278, WO-A-03020721).

The prior art describes potent in vitro inhibitors, but also highlights the many difficulties in developing a human therapeutic. For example, WO-A-9850533 and WO-A-0029408 describe compounds that may be referred to as cyclic ketones and are inhibitors of cysteine proteinases with a particular reference towards papain family proteinases and as a most preferred embodiment, cathepsin K. WO-A-9850533 describes compounds subsequently detailed in the literature as potent inhibitors of cathepsin K with good oral bioavailability (Witherington, J., ‘Tetrahydrofurans as Selective Cathepsin K Inhibitors’, RSC meeting, Burlington House, London, 1999). The compounds of WO-A-9850533 were reported to bind to cathepsin K through the formation of a reversible covalent bond between the tetrahydrofuran carbonyl and the active site catalytic cysteine residue (Witherington, J., 1999).

Additionally, the same cyclic ketone compounds are described in WO-A-9953039 as part of a wide-ranging description of inhibitors of cysteine proteinases associated with parasitic diseases, with particular reference to the treatment of malaria by inhibition of falcipain. However, subsequent literature describes the cyclic ketone compounds of WO-A-9850533 to be unsuitable for further development or for full pharmacokinetic evaluation due to a physiochemical property of the inhibitors, the poor chiral stability of the α-aminoketone chiral centre (Marquis, R. W. et al, J. Med. Chem., 44(5). 725-736, 2001). WO-A-0069855 describes compounds that may also be referred to as cyclic ketones with particular reference towards inhibition of cathepsin S.

The compounds of WO-A-0069855 are considered to be an advance on compounds of WO-A-9850533 due to the presence of the β-substituent on the cyclic ketone ring system that provides chiral stability to the α-carbon of the cyclic ketone ring system. However, the compounds of WO-A-0069855 and indeed those of WO-A-9850533 describe a requirement for the presence of the potential hydrogen-bonding motif X—NHCHRCO—NH—Y that is widely observed amongst the prior art substrate-based inhibitors of CAC1 proteinases. Additionally, within these substrate-based inhibitors, the central X—NHCHRCO—NH—Y part of the inhibitor motif occupies the S2 binding pocket of the proteinase and is considered to be an indispensable feature for achieving potency and selectivity for the inhibitors.

Our earlier patent applications (WO-A-02057270, WO-A-04007501) describe bicyclic compounds in which the chirality of the α-aminoketone is stabilised (for a review of energetic considerations within fused ring systems see (a) Toromanoff, E. Tetrahedron Report No 96, X, 2809-2931, 1980; (b) Eliel, E. L. et. al. Stereochemistry of Organic Compounds, Wiley: New York, 1-1267, 1994). These compounds do not contain the X—NHCHRCO—NH—Y motif and yet the compounds are highly potent inhibitors across a broad range of CAC1 cysteine proteinases. However, bicyclic compounds of our earlier patent applications (WO-A-02057270, WO-A-04007501) retained the central X—NHCHRCO—NH—Y part of the inhibitor motif that occupies the S2 binding pocket of the proteinase and this is considered to be an indispensable feature for achieving potency and selectivity for inhibitor compounds. In particular, certain of the compounds are potent and selective inhibitors of a range of mammalian and parasitic CAC1 proteinases.

The present inventors have now discovered new bicyclic compounds that additionally diverge from the central X—NHCHRCO—NH—Y part of the motif that was previously believed to be an indispensable feature for achieving potency and selectivity for inhibitor compounds. The compounds of the present invention no longer contain this central motif and yet surprisingly the compounds are highly potent inhibitors across a broad range of CAC1 cysteine proteinases. In particular, some compounds of the present invention are potent and selective inhibitors of cathepsin K.

### STATEMENT OF INVENTION

A first aspect of the invention relates to compounds of general formula (I), and pharmaceutically acceptable salts thereof,

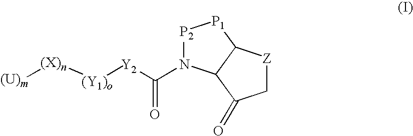

wherein:
Z is O,

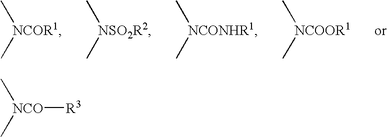

where R¹ and R² are each independently a hydrocarbyl group, and R³ is a saturated heterocycle defined by

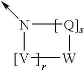

where
Q and V are each independently selected from

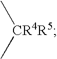

W is selected from

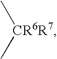

O, S,

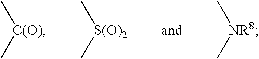

‘r’ and ‘s’ are each independently 1 or 2;
P₁ is

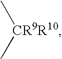

where R⁹ and R¹⁰ are each independently selected from H, alkyl, cycloalkyl, Ar-alkyl, Ar, halogen, alkoxy, hydroxyl and NR⁴⁶R⁴⁷, wherein R⁴⁶ and R⁴⁷ are each independently H or alkyl;
P₂ is O,

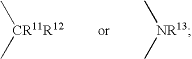

Y₂ is O, S or

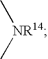

or where (U)ₘ, (X)ₙ and (Y₁)ₒ are absent, Y₂ is OR⁴⁸, SR⁴⁸ or —NR¹⁴R⁴⁴, where R⁴⁸ is alkyl, and R¹⁴ and R⁴⁴ are each independently selected from H and alkyl, or R¹⁴ and R⁴⁴ are linked to form a cyclic group together with the nitrogen to which they are attached;
each Y₁ is independently

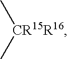

and ‘o’ is 0, 1, 2 or 3;
or when ‘o’ is 1, Y₁ may additionally be selected from

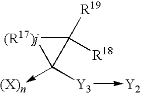

where Y₃ is methylene or absent;
R¹⁷ is selected from

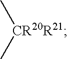

‘j’ is 1, 2, 3 or 4, where when ‘j’ is 2, 3 or 4, R¹⁷ may additionally be selected from O, S, O₂, NR²² and —N(R²²)C(O)—;
or when ‘o’ is 1, 2, or 3 and (U)ₘ and (X)ₙ are absent, the terminal Y₁ group is selected from CR¹⁵R¹⁶R⁴² and

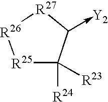

R²⁵ is selected from

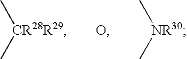

R²⁶ is selected from

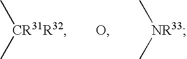

except when R²⁵ is O, then R²⁶ is selected from

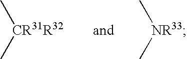

R²⁷ is selected from

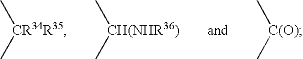

each X is independently

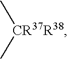

O, S,

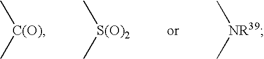

‘n’ is 0, 1 or 2, provided that when (Y₁)ₒ is absent, (X)ₙ is CR³⁷R³⁸ or is absent, and also provided that when ‘n’ is 2, (X)ₙ contains a minimum of one

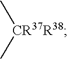

and when (U)ₘ is absent and n is 1 or 2, the terminal X group is CR³⁷R³⁸R⁴³;
each U is independently a 5- to 7-membered monocyclic or a 8- to 11-membered bicyclic ring which is either saturated or unsaturated and which includes up to four heteroatoms as shown below:

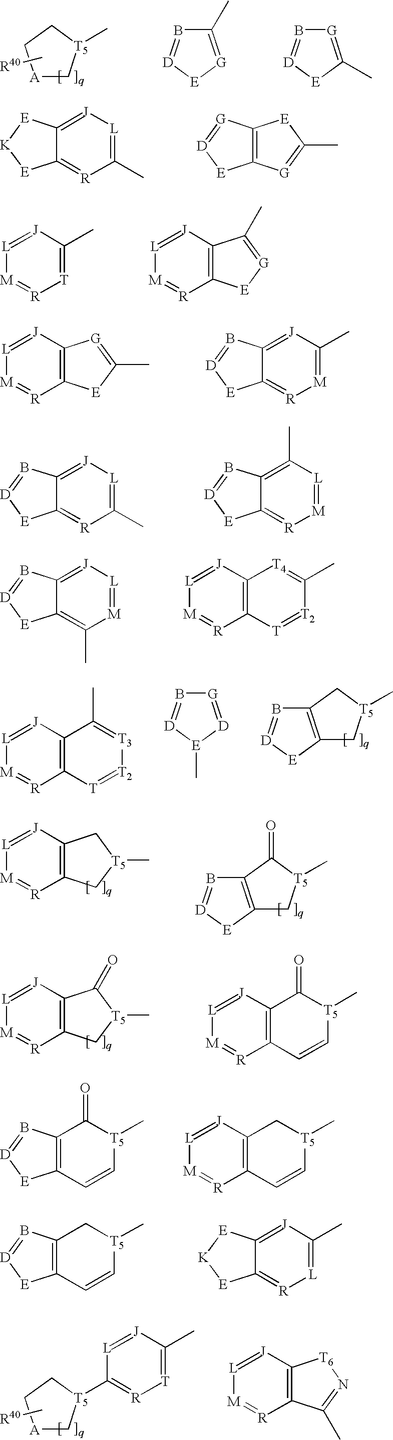

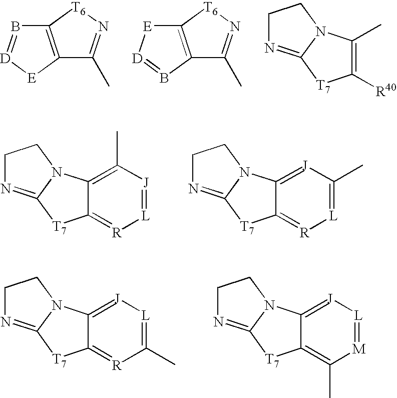

wherein R⁴⁰ is:
   H, haloalkyl, alkyl, cycloalkyl, Ar-alkyl, Ar, OH, O-alkyl, O-cycloalkyl, O-allyl, OAr, S-alkyl, SH, S-cycloalkyl, S—Ar-alkyl, SAr, SO₂-alkyl, NHCO-alkyl, SO₂H, SO₂-cycloalkyl, SO₂—Ar-alkyl, SO₂Ar, NH-alkyl, NH₂, NH-cycloalkyl, NH—Ar-alkyl, NHAr, N(alkyl)₂, NH₂, NH(alkyl), N(cycloalkyl)₂ or N(Ar-alkyl)₂ or NAr₂; or, when part of a CHR⁴⁰ or CR⁴⁰ group, R⁴⁰ may be halogen;
A is selected from:
   CH₂,

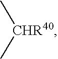

O, s,

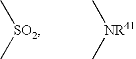

and N-oxide

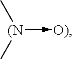

where R⁴⁰ is as defined above; and R⁴¹ is selected from H, alkyl, cycloalkyl, Ar and Ar-alkyl;
B, D and G are each independently selected from:

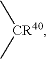

where R⁴⁰ is as defined above, N and N-oxide

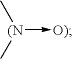

E is selected from:
   CH₂,

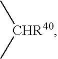

O, S,

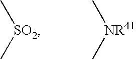

and N-oxide

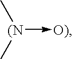

where R⁴⁰ and R⁴¹ are defined as above;
K is selected from:
   CH₂,

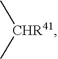

where R⁴¹ is defined as above;
J, L, M, R, T, T₂, T₃ and T₄ are independently selected from:
   CR⁴⁰ where R⁴⁰ is as defined above, N and N-oxide

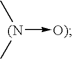

T₅ is selected from:
   CH and N;
T₆ is selected from:

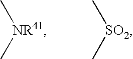

OC(O),

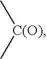

and N(r)⁴¹)C(O);
T₇ is selected from:

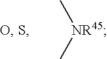

‘q’ is 1, 2 or 3;
‘m’ is 0 or 1;
R⁴⁻⁷, R¹¹⁻¹², R¹⁵⁻¹⁶, R¹⁸⁻²¹, R²³⁻²⁴, R²⁸⁻²⁹, R³¹⁻³², R³⁴⁻³⁵, R³⁷⁻³⁸ and R⁴²⁻⁴³ are each independently selected from H, alkyl, cycloalkyl, Ar-alkyl, Ar and halogen; and R⁸, R¹³, R²², R³⁰, R³³, R³⁶, R³⁹ and R⁴⁵ are each independently selected from H, alkyl, cycloalkyl, Ar-alkyl and Ar.

A second aspect of the invention relates to a pharmaceutical or veterinary composition comprising a compound of formula (I) and a pharmaceutically acceptable or veterinarily acceptable diluent, excipient and/or carrier.

A third aspect of the invention relates to a process for preparing a pharmaceutical or veterinary composition as defined above, said process comprising admixing a compound of the invention with a pharmaceutically acceptable or veterinarily acceptable diluent, excipient and/or carrier.

A fourth aspect of the invention relates to compound of formula (I) for use in medicine.

A fifth aspect of the invention relates to the use of a compound of formula (I) in the preparation of a medicament for treating a disease selected from osteoporosis, Paget's disease, Chagas's disease, malaria, gingival diseases, hypercalaemia, metabolic bone disease and diseases involving matrix or cartilage degradation.

A sixth aspect of the invention relates to a method of inhibiting a cysteine proteinase in a cell, said method comprising contacting said cell with a compound of formula (X).

A seventh aspect of the invention relates to method of inhibiting a cysteine proteinase in a subject, said method comprising administering to the subject a pharmacologically effective amount of a compound of formula (I).

An eighth aspect of the invention relates to a method of treating a disease selected from osteoporosis, Paget's disease, Chagas's disease, malaria, gingival diseases, hypercalaemia, metabolic bone disease and diseases involving matrix or cartilage degradation, in a subject, said method comprising administering to the subject a pharmacologically effective amount of a compound of formula (I).

A ninth aspect of the invention relates to the use of a compound according to the invention in an assay for identifying further candidate compounds capable of inhibiting one or more cysteine proteinases.

A tenth aspect of the invention relates to the use of a compound of formula (I) in the validation of a known or putative cysteine proteinase as a therapeutic target.

An eleventh aspect of the invention relates to a process of preparing a compound of formula I.

### DETAILED DESCRIPTION

As used herein, the term “hydrocarbyl” refers to a group comprising at least C and H. If the hydrocarbyl group comprises more than one C then those carbons need not necessarily be linked to each other. For example, at least two of the carbons may be linked via a suitable element or group. Thus, the hydrocarbyl group may contain heteroatoms. Suitable heteroatoms will be apparent to those skilled in the art and include, for instance, sulphur, nitrogen, oxygen, phosphorus and silicon. Where the hydrocarbyl group contains one or more heteroatoms, the group may be linked via a carbon atom or via a heteroatom to another group, i.e. the linker atom may be a carbon or a heteroatom. Preferably, the hydrocarbyl group is an aryl, heteroaryl, alkyl, cycloalkyl, aralkyl, heterocycloalkyl, or alkenyl group. More preferably, the hydrocarbyl group is an aryl, heteroaryl, alkyl, cycloalkyl or aralkyl group. Suitable substituents include, for example, one or more R⁴⁰, NO₂, CN, CF₃ and/or halo groups.

‘Halogen’ as applied herein encompasses F, Cl, Br, I.

‘Heteroatom’ as applied herein encompasses O, S, P and N, more preferably, O, S and N.

The term ‘alkyl’ as applied herein includes stable straight and branched chain aliphatic carbon chains which may be optionally substituted. Preferred examples include methyl, ethyl, n-propyl, isopropyl, n-butyl, isobutyl, t-butyl, pentyl, isopentyl, hexyl, heptyl and any simple isomers thereof. Suitable substituents include, for example, one or more R⁴⁰, NO₂, CN, CF₃ and/or halo groups. Additionally, where the alkyl group contains two or more contiguous carbon atoms, an alkene group (—CH═CH—) or alkyne group (—C≡C—) may be present. Furthermore, the alkyl group may optionally contain one or more heteroatoms (as defined above) for example, to give ethers, thioethers, sulphones, sulphonamides, substituted amines, amidines, guanidines, carboxylic acids, carboxamides. If the heteroatom is located at a chain terminus then it is appropriately substituted with one or two hydrogen atoms. For example, the group CH₃—CH₂—O—CH₂—CH₂— is defined within ‘alkyl’ as a C₄ alkyl that contains a centrally positioned heteroatom whereas the group CH₃—CH₂—CH₂—CH₂— is defined within ‘alkyl’ as an unsubstituted C₄ alkyl.

Preferably, the alkyl group is a C₁₋₂₀ alkyl group, more preferably a C₁₋₁₅ group, even more preferably a C₁₋₁₂ alkyl group, more preferably still, a C₁₋₄ alkyl group, more preferably a C₁₋₃ alkyl group.

As used herein, the term “cycloalkyl” refers to a cyclic alkyl group (i.e. a carbocyclic ring) which may be substituted (mono- or poly-) or unsubstituted. Suitable substituents include, for example, one or more R⁴⁰, NO₂, CN, CF₃ and/or halo groups. Preferably, the cycloalkyl group is a C₃₋₁₂ cycloalkyl group, more preferably a C₃₋₆-cycloalkyl. Examples include cyclopropyl, cyclobutyl, cyclopentyl, cyclohexyl and the like. In addition, the carbocyclic ring itself may optionally contain one or more heteroatoms, for example, to give a heterocycloalkyl group such as tetrahydrofuran, pyrrolidine, piperidine, piperazine or morpholine.

As used herein, the term “aryl” or “Ar” refers to a C₆₋₁₂ aromatic group which may be substituted (mono- or poly-) or unsubstituted. Preferably, the aromatic group is a stable 5 or 6-membered monocyclic or a stable 8 to 10 membered bicyclic ring which is unsaturated. Typical examples include phenyl and naphthyl etc. Suitable substituents include, for example, one or more R⁴⁰, NO₂, CN, CF₃ and/or halo groups.

As used herein, the term “heteroaryl” refers to a C₂₋₁₂ aromatic, substituted (mono- or poly-) or unsubstituted group, which comprises one or more heteroatoms. Preferably, the heteroaryl group is a C₄₋₁₂ aromatic group comprising one or more heteroatoms selected from N, O and S. Suitable heteroaryl groups include pyrrole, pyrazole, pyrimidine, pyrazine, pyridine, quinoline, thiophene, 1,2,3-triazole, 1,2,4-triazole, thiazole, oxazole, iso-thiazole, iso-oxazole, imidazole, furan and the like. Suitable substituents include, for example, one or more R⁴⁰, NO₂, CN, CF₃ and/or halo groups.

As used herein, the term “aralkyl” or “Ar-alkyl” includes, but is not limited to, a group having both aryl and alkyl functionalities, which may be optionally substituted by one or more R⁴⁰, NO₂, CN, CF₃ and/or halo groups. By way of example, the term includes groups in which one of the hydrogen atoms of the alkyl group is replaced by an aryl group, e.g. a phenyl group. Typical aralkyl groups include benzyl, phenethyl and the like.

The present invention includes all salts, hydrates, solvates, complexes and prodrugs of the compounds of this invention. The term “compound” is intended to include all such salts, hydrates, solvates, complexes and prodrugs, unless the context requires otherwise.

Abbreviations and symbols commonly used in the peptide and chemical arts are used herein to describe compounds of the present invention, following the general guidelines presented by the IUPAC-IUB Joint Commission on Biochemical Nomenclature as described in Eur. J. Biochem., 15, 9-, 1984. Compounds of formula (I) and the intermediates and starting materials used in their preparation are named in accordance with the IUPAC rules of nomenclature in which the characteristic groups have decreasing priority for citation as the principle group.

In a preferred embodiment of the invention, R¹ and R² are each independently selected from alkyl, cycloalkyl, Ar-alkyl and Ar, each of which may be optionally substituted by one or more R⁴⁰, NO, CN, CF₃ and/or halo groups.

In a preferred embodiment of the invention, R¹ is selected from alkyl and aryl, each of which may be optionally substituted by one or more R⁴⁰, NO₂, CN, CF₃ and/or halo groups.

In a preferred embodiment of the invention, P¹ is

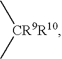

where R⁹ and R¹⁰ are each independently selected from H, alkyl, cycloalkyl, Ar-alkyl, Ar, halogen, alkoxy and NR⁴⁶R⁴⁷, wherein R⁴⁶ and R⁴⁷ are each independently H or alkyl.

In one preferred embodiment of the invention, P₁ is

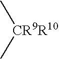

and R⁹ and R¹⁰ are each independently H, alkyl, alkoxy, NR⁴⁶R⁴⁷ or halogen.

Preferably, P₁ is CH-halogen, CH₂, CH(OMe), CH(NH₂) or CH(NHMe).

In one particularly preferred embodiment, P₁ is CH₂.

In one preferred embodiment of the invention, P₂ is

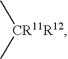

O or NR¹³, and R¹¹⁻¹³ are each independently H or alkyl.

More preferably, P₂ is CH₂, O or NH.

Even more preferably, P₂ is CH₂.

In one preferred embodiment of the invention, Z is O or NCOR¹.

In one preferred embodiment of the invention, Z is O.

In one preferred embodiment of the invention, Z is NCOR¹.

In a more preferred embodiment of the invention, Z is O or NCOAr.

In an even more preferred embodiment of the invention, Z is O or NCOPh.

In one preferred embodiment of the invention, Y₂ is O, NH or S.

In one preferred embodiment of the invention, Y₁ is

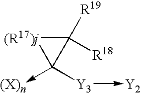

In a more preferred embodiment of the invention, R¹⁷ is CH₂, j is 2 and R¹⁹ and R¹⁸ are both H.

In one preferred embodiment of the invention, Y₃ is absent.

In one preferred embodiment of the invention, R¹⁷ is CH₂, j is 2 and R¹⁹ and R¹⁸ are both H and Y₃ is absent.

In another preferred embodiment of the invention, R¹⁷ is CH₂, j is 2 and R¹⁹ and R¹⁸ are both H and Y₃ is CH₂.

In one preferred embodiment of the invention, R¹⁷ is CH₂, j is 1 and R¹⁹ and R¹⁸ are both H and Y₃ is absent.

In another preferred embodiment of the invention, R¹⁷ is CH₂, j is I and R¹⁹ and R¹⁸ are both H and Y₃ is CH₂.

In one preferred embodiment of the invention, Y₁ is CR¹⁵R¹⁶ and o is 0, 1, 2 or 3.

In one preferred embodiment of the invention, (Y₁)ₒ is cyclobutyl and o is 1.

In one preferred embodiment of the invention, (Y₁)ₒ is cyclopropyl and o is 1.

In another preferred embodiment, (Y₁)ₒ is CHEt or CHⁱPr and o is 1.

In another preferred embodiment, (Y₁)ₒ is CH₂ and o is 1.

In another preferred embodiment, (Y₁)ₒ is CHEtOH₂, i.e. o is 2, where one Y₁ is CHEt and the other is CH₂.

In one preferred embodiment of the invention, X is CR³⁷R³¹.

In one preferred embodiment of the invention, (X)ₙ is CH₂O, i.e. n is 2, where one X is CH₂ and the other is O.

In another preferred embodiment of the invention, n is 1 and X is O.

In another preferred embodiment, (X)ₙ is CH₂ and n is 1 or 2.

In another preferred embodiment, (X)ₙ is CHEt and n is 1.

In another preferred embodiment, (X)ₙ is CHEtCH₂, i.e. n is 2, where one X is CHEt and the other is CH₂.

In one preferred embodiment R⁴⁰ is H, alkyl, cycloalkyl, Ar-alkyl, Ar, OH, O-alkyl, O-cycloalkyl, O-alkyl, OAr, S-alkyl, SH, S-cycloalkyl, S—Ar-alkyl, SAr, SO₂-alkyl, NHCO-alkyl, SO₂H, SO₂-cycloalkyl, SO₂—Ar-alkyl, SO₂Ar, NH-alkyl, NH₂, NH-cycloalkyl, NH—Ar-alkyl, NHAr, N(alkyl)₂, NH₂, NH(alkyl), N(cycloalkyl)₂ or N(Ar-alkyl)₂ or NAr₂; or, when part of a CHR⁴⁰ or CR⁴⁰ group, R⁴⁰ may be halogen;

In a more preferred embodiment of the invention, U is

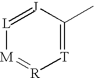

and J, L, M, R and T are each independently CR⁴⁰.

In one highly preferred embodiment of the invention, U is phenyl and m is 1.

In one preferred embodiment, m is 1 and J, L, M, R and T are each independently CR⁴⁰, wherein each R⁴⁰ is independently selected from H, alkyl, halo, alkoxy and haloalkyl.

In a more preferred embodiment, m is 1, J, L, M, R and T are each independently CRC, wherein each R⁴⁰ is independently selected from H, methyl, ethyl, Cl, F, ethoxy, isopropyloxy and CF₃.

In a further preferred embodiment, m is 1, one of J, L, M, R and T is N and the remainder are each independently CR⁴⁰. Preferably, each R⁴⁰ is independently selected from H, alkyl, halo, alkoxy and haloalkyl, more preferably, H, methyl, ethyl, Cl, F, ethoxy, isopropyloxy and CF₃.

In one highly preferred embodiment, J is N and L, M, R and T are each independently R⁴⁰.

In another preferred embodiment of the invention, m is 1 and U is

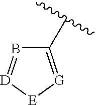

Preferably, E is S and B, D and G are each independently CR⁴⁰. More preferably, E is S and B, D and G are all CH.

In a further preferred embodiment, m is 1, and U is selected from the following:

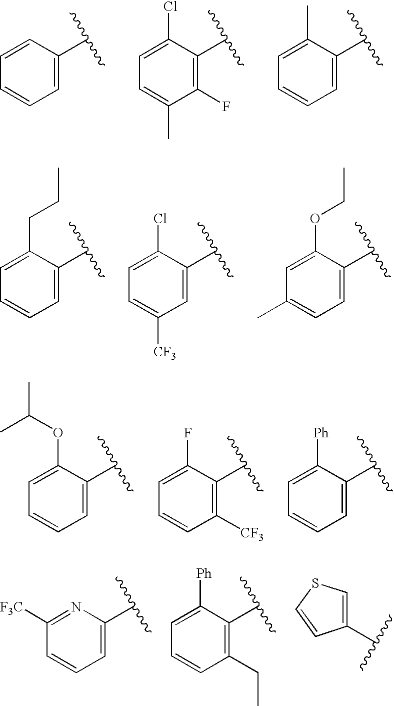

In one preferred embodiment of the invention, P₂ is

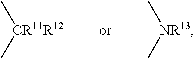

and the stereochemistry is (3aS,6aR) or (3aR,6aS).

In another preferred embodiment of the invention, P₂ is O, and the stereochemistry is (3aS,6aS) or (3aR,6aR).

In another preferred embodiment of the invention, P₂ is

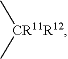

Z is O and the stereochemistry is (3aS,6aR).

In another preferred embodiment of the invention, P₂ is O, Z is O, and the stereochemistry is (3aS,6aS).

In another preferred embodiment of the invention, P₂ is

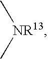

Z is O, and the stereochemistry is (3aR,6aS).

In another preferred embodiment of the invention, P₂ is

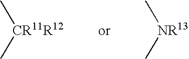

### and Z is

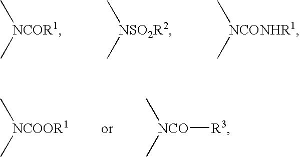

and the stereochemistry is (3aR,6aS).

In another preferred embodiment of the invention, P₂ is O, Z is

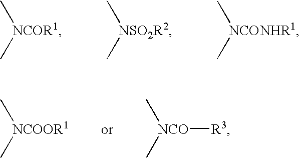

and the stereochemistry is (3aS,6aS).

In one particularly preferred embodiment of the invention, m is 0, (X)ₙ is CR³⁷R³⁸R⁴³ and n is 1.

In an even more preferred embodiment of the invention, n is 1, m is 0 and X is CH₃, CH(alkyl)₂ or C(alkyl)₃. More preferably still, n is 1, m is 0 and X is CH₃, CH₂Me, CH(Me)₂ or CMe₃.

In one preferred embodiment of the invention, o is 1 or 2 and each Y₁ is independently

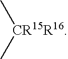

More preferably, R¹⁵ and R¹⁶ are each independently H or alkyl.

In one particularly preferred embodiment of the invention, (Y₁)ₒ is CHⁱPr, CHMe, CH₂, or CH(Me)CH₂.

In another preferred embodiment of the invention, ‘o’ is 1, 2, or 3, (U)ₘ and (X)ₙ are absent, and the terminal Y₁ group is

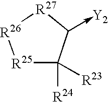

More preferably, R²⁷ is CO, R²⁶ is O, R²⁵ is CH₂ and R²³ and R²⁴ are both CH₃.

Even more preferably still, o is 1.

In another preferred embodiment of the invention, (U)ₘ, (X)ₙ and (Y₁)ₒ are absent, and Y₂ is —NR¹⁴R⁴⁴, where R¹⁴ and R⁴⁴ are linked to form a cyclic group together with the nitrogen to which they are attached.

In one highly preferred embodiment of the invention, R¹⁴ and R⁴⁴ are linked to together with the nitrogen to which they are attached to form a pyrrolidine group.

In another particularly preferred embodiment of the invention, R¹ is alkyl optionally substituted by one or more NHCO-alkyl groups.

Even more preferably, R¹ is

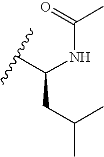

One preferred embodiment of the invention relates to a compound of formula (I) in which Z is O, P₁, P₂ are methylene, Y₂ is O, (Y₁)ₒ is cyclobutyl, Y₃ is absent, ‘o’ is one, (X)ₙ is O and —CH₂— combining to form —OCH₂—, ‘n’ is two, (U)ₘ is phenyl and ‘m’ is one, thus named:—

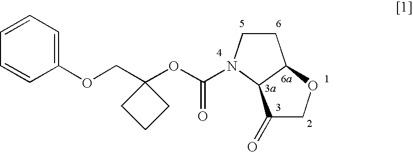

### (3aS,6aR)-3-Oxo-hexahydro-furo[3,2-b]pyrrole-4-carboxylic acid 1-phenoxymethyl-cyclobutyl ester [1]

Another preferred embodiment of the invention relates to a compound of formula (I) in which Z is O, P₁, P₂ are methylene, Y₂ is NH, (Y₁)ₒ is cyclobutyl, Y₃ is absent, ‘o’ is one, (X)ₙ is O and —CH₂— combining to form —OCH₂—, ‘n’ is two, (U)ₘ is phenyl and ‘m’ is one, thus named:—

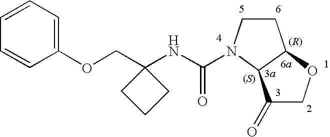

### [2](3aS,6aR)-3-Oxo-hexahydro-furo[3,2-b]pyrrolecarboxylic acid (1-phenoxymethyl-cyclobutyl)-amide [2]

Another preferred embodiment of the invention relates to a compound of formula (I) in which Z is O, P₁, P₂ are methylene, Y₂ is S, (Y₁)ₒ is cyclobutyl, Y₃ is absent, ‘o’ is one, (X)ₙ is O and —CH₂— combining to form —OCH₂—, ‘n’ is two, (U)ₘ is phenyl and ‘m’ is one, thus named:—

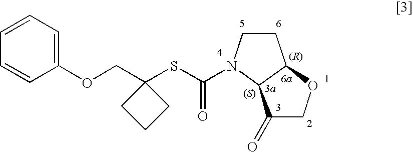

### (3aS,6aR)-3-oxo-hexahydro-furo[3,2-b]pyrrole-4-carbothioic acid S-(1-phenoxy methyl-cyclobutyl)ester [3]

Another preferred embodiment of the invention relates to a compound of formula (I) in which Z is O, P₁ is methylene, P₂ is O Y₂ is O, (Y₁)ₒ is cyclobutyl, Y₃ is absent, ‘o’ is one, (X)ₙ is O and —CH₂— combining to form —OCH₂—, ‘n’ is two, (U)ₘ is phenyl and ‘m’ is one, thus named:—

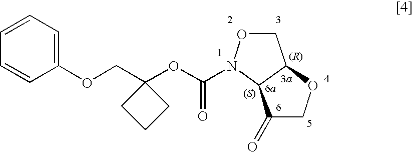

### (3aS,6aS)-6-Oxo-tetrahydro-furo[3,2-c]isoxazole-1-carboxylic acid 1-phenoxymethyl-cyclobutyl ester [4]

Yet another preferred embodiment of the invention relates to a compound of formula (I) in which Z is O, P₁ is methylene, P₂ is NH, Y₂ is O, (Y₁)ₒ is cyclobutyl, Y₃ is absent, ‘o’ is one, (X)ₙ is O and —CH₂— combining to form —OCH₂—, ‘n’ is two, (U)ₘ is phenyl and ‘m’ is one, thus named:—

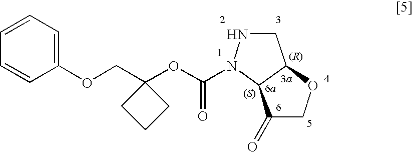

### (3aR,6aS)-6-Oxo-hexahydro-furo[3,2-c]pyrazole-1-carboxylic acid 1-phenoxymethyl-cyclobutyl ester [5]

Another preferred embodiment of the invention relates to a compound of formula (I) in which Z is

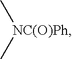

P₁, P₂ are methylene, Y₂ is O, (Y₁)ₒ is cyclobutyl, Y₃ is absent, ‘o’ is one, (X)ₙ is O and —CH₂— combining to form —OCH₂—, ‘n’ is two, (U)ₘ is phenyl and ‘m’ is one, thus named:—

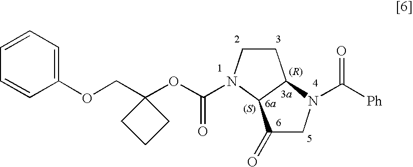

### (3aR,6aS)-4-Benzoyl-6-oxo-hexahydro-pyrrolo[3,2-b]pyrrole-1-carboxylic acid 1-phenoxymethyl-cyclobutyl ester [6]

Another preferred embodiment of the invention relates to a compound of formula (I) in which Z is

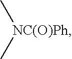

P₁ is methylene, P₂ is O, Y₂ is O, Y₂ is O, (Y₁)ₒ is cyclobutyl, Y₃ is absent, ‘o’ is one, (X)ₙ is O and —CH₂— combining to form —OCH₂—, ‘n’ is two, (U)ₘ is phenyl and ‘m’ is one, thus named:—

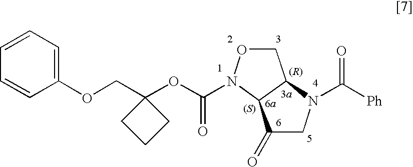

### (3aS,6aS)-4-Benzoyl-oxo-hexahydro-2-oxa-1,4-diaza-pentalene-1-carboxylic acid 1-phenoxymethyl-cyclobutyl ester [7]

Another preferred embodiment of the invention relates to a compound of formula (I) in which Z is

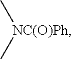

P₁ is methylene, P₂ is NH, Y₂ is O, (Y₁)ₒ is cyclobutyl, Y₃ is absent, ‘o’ is one, (X)ₙ is O and —CH₂— combining to form —OCH₂—, ‘n’ is two, (U)ₘ is phenyl and ‘m’ is one, thus named:—

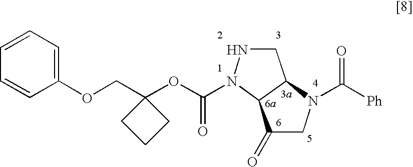

### (3aR,6aS)-4-Benzoyl-6-oxo-hexahydro-pyrrolo[3,2-c]pyrazole-1-carboxylic acid 1-phenoxymethyl-cyclobutyl ester [8]

In one especially preferred embodiment of the invention, the compound of formula (I) is selected from the following:
(3aS,6aR)-3-Oxo-hexahydro-furo[3,2-b]pyrrole-4-carboxylic acid 1-phenoxymethyl-cyclobutyl ester [1];
(3aS,6aR)-3-Oxo-hexahydro-furo[3,2-b]pyrrole-4-carboxylic acid (1-phenoxymethyl-cyclobutyl)-amide [2];
(3aS,6aR)-3-Oxo-hexahydro-furo[3,2-b]pyrrole-4-carbothioic acid S-(1-phenoxy methyl-cyclobutyl)ester [3];
(3aS,6aS)-6-Oxo-tetrahydro-furo[3,2-]isoxazole-1-carboxylic acid 1-phenoxymethyl-cyclobutyl ester [4];
(3aR,6aS)-6-Oxo-hexahydro-furo[3,2-c]pyrazole-1-carboxylic acid 1-phenoxymethyl -cyclobutyl ester [5];
(3aR,6aS)-4-Benzoyl-6-oxo-hexahydro-pyrrolo[3,2-b]pyrrole-1-carboxylic acid 1-phenoxymethyl-cyclobutyl ester [6];
(3aS,6aS)-4-Benzoyl-6-oxo-hexahydro-2-oxa-1,4-diaza-pentalene-1-carboxylic acid 1-phenoxymethyl-cyclobutyl ester [7];
(3aR,6aS)-4-Benzoyl-6-oxo-hexahydro-pyrrolo[3,2-c]pyrazole-1-carboxylic acid 1-phenoxymethyl-cyclobutyl ester [8];
(3aR,6aS)-4-Benzoyl-6-oxo-hexahydro-pyrrolo[3,2-b]pyrrole-1-carboxylic acid 1-isopropyl-2-methyl-propyl ester [9];
(3aR,6aS)-4-(2S-Acetylamino-4-methyl-pentanoyl)-6-oxo-hexahydro-pyrrolo[3,2-b]pyrrole-1-carboxylic acid 1-isopropyl-2-methyl-propyl ester [10];
(3aR,6aS)-4-(2S-Acetylamino-4-methyl-pentanoyl)-6-oxo-hexahydro-pyrrolo[3,2-b]pyrrole-1-carboxylic acid isobutyl ester [11];
(3aR,6aS)-4-(2S-Acetylamino-4-methyl-pentanoyl)-6-oxo-hexahydro-pyrrolo[3,2-b]pyrrole-1-carboxylic acid isopropyl ester [12];
(3aR,6aS)-4-(2S-Acetylamino-4-methyl-pentanoyl)-6-oxo-hexahydro-pyrrolo[3,2-b]pyrrole-1-carboxylic acid 2,2-dimethyl-propyl ester [1,3];
(3aR,6aS)-4-(2S-Acetylamino-4-methyl-pentanoyl)-6-oxo-hexahydro-pyrrolo[3,2-b]pyrrole-1-carboxylic acid diethylamide [14];
(3aR,6aS)-4-(2S-Acetylaminomethyl-pentanoyl)-6-oxo-hexahydro-pyrrolo[3,2-b]pyrrole-1-carboxylic acid sec-butylamide [15];
(3aR,6aS)—N-(3-Methyl-1-[3-oxo-4-(pyrrolidine-1-carbonyl)-hexahydro-pyrrolo[3,2-b]pyrrole-1-carbonyl]-butyl)-acetamide [16];
(3aR,6aS)-4-Benzoyl-6-oxo-hexahydro-pyrrolo[3,2-b]pyrrole-1-carboxylic acid 4,4-dimethyl-2-oxo-tetrahydro-furan-3R-yl ester [17];
(3aR,6aS)-4-Benzoyl-6-oxo-hexahydro-pyrrolo[3,2-b]pyrrole-1-carboxylic acid 4,4-dimethyl-2-oxo-tetrahydro-furan-3S-yl ester [18];
(3aS,6aR)-3-Oxo-hexahydro-furo[3,2-b]pyrrole-4-carboxylic acid 2,2-dimethyl-propyl ester [19];
(3aR,6aS)-1-Benzylcyclobutyl 4-benzoyl-6-oxohexahydropyrrolo[3,2-b]pyrrole-1(2H)-carboxylate;
(3aR,6aS)-1-Phenethylcyclobutyl 4-benzoyl-6-oxohexahydropyrrolo[3,2-b]pyrrole 1(2H)-carboxylate;
(3aR,6aS)-1-(Thiophen-3-yl)butan-2-yl 4-benzoyl-6-oxohexahydropyrrolo[3,2-b]pyrrole-1(2H)-carboxylate;
(3aR,6aS)-(1-(Phenoxymethyl)cyclobutyl)methyl 4-benzoyl-6-oxohexahydropyrrolo[3,2-b]pyrrole-1(2H)-carboxylate;
(3aR,6aS)-1-(Thiophen-2-yl)butan-2-yl 4-benzoyl-6-oxohexahydropyrrolo[3,2-b]pyrrole-1(2H)-carboxylate;
(3aR,6aS)-1-Isopropylcyclopropyl 4-benzoyl-6-oxohexahydropyrrolo[3,2-b]pyrrole-1(2H)-carboxylate;
(3aR,6aS)-5-Methyl-1-(thiophen-2-yl)hexan-3-yl 4-benzoyl-6-oxohexahydropyrrolo[3,2-b]pyrrole-1(2H)-carboxylate;
(3aR,6aS)-5,5-Dimethylhexan-3-yl 4-benzoyl-6-oxohexahydropyrrolo[3,2-b]pyrrole-1(2H)-carboxylate;
(3aR,6aS)-3-Methyl-1-phenylbutyl 4-benzoyl-6-oxohexahydropyrrolo[3,2-b]pyrrole-1(2H)-carboxylate;
(3aS,6aR)-1-Benzylcyclobutyl 3-oxotetrahydro-2H-furo[3,2-b]pyrrole-4(5H)-carboxylate;
(3aS,6aR)-1-Phenethylcyclobutyl 3-oxotetrahydro-2H-furo[3,2-b]pyrrole-4(5H)-carboxylate;
(3aS,6aR)-1-(Thiophen-3-yl)butan-2-yl 3-oxotetrahydro-2H-furo[3,2-b]pyrrole-4(5H)-carboxylate;
(3aS,6aR)-(1-(Phenoxymethyl)cyclobutyl)methyl 3-oxotetrahydro-2H-furo[3,2-b]pyrrole-4(5H)-carboxylate;
(3aS,6aR)-1-(Thiophen-2-yl)butan-2-yl 3-oxotetrahydro-2H-furo[3,2-b]pyrrole-4(5H)-carboxylate;
(3aS,6aR)-1-Isopropylcyclopropyl 3-oxotetrahydro-2H-furo[3,2-b]pyrrole-4(5H)-carboxylate;
(3aS,6aR)-5-Methyl-1-(thiophen-2-yl)hexan-3-yl 3-oxotetrahydro-2H-furo[3,2-b]pyrrole-4(5H)-carboxylate;
(3aS,6aR)-3-Methyl-1-phenylbutyl 3-oxotetrahydro-2H-furo[3,2-b]pyrrole-4(5H)-carboxylate;
(3aS,6aR)-5,5-Dimethylhexan-3-yl 3-oxotetrahydro-2H-furo[3,2-b]pyrrole-4(5H)-carboxylate;
(3aS,6aR)-4-Ethylbiphenyl-3-yl 3-oxotetrahydro-2H-furo[3,2-b]pyrrole-4(5H)-carboxylate;
(3aR,6aS)-4-Benzoyl-6-oxo-N-(1-(thiophen-3-yl)butan-2-yl)hexahydropyrrolo[3,2-b]pyrrole-1(2H)-carboxamide;
(3aR,6aS)-6-Oxo-4-(pyrrolidine-1-carbonyl)-N-(1-(thiophen-3-yl)butan-2-yl)hexahydropyrrolo[3,2-b]pyrrole-1(2H)-carboxamide;
(3aR,6aS)-4-Benzoyl-6-oxo-N-(1-(thiophen-2-yl)butan-2-yl)hexahydropyrrolo[3,2-b]pyrrole-1(2H)-carboxamide;
(3aR,6aS)-6-Oxo-4-(pyrrolidine-1-carbonyl)-N-(1-(thiophen-2-yl)butan-2-yl)hexahydropyrrolo[3,2-b]pyrrole-1(2H)-carboxamide;
(3aR,6aS)-4-Benzoyl-6-oxo-N-((1-(phenoxymethyl)cyclobutyl)methyl)hexahydropyrrolo[3,2-b]pyrrole-1(2H)-carboxamide;
(3aR,6aS)-4-Benzoyl-N-(5-methyl-1-(thiophen-2-yl)hexan-3-yl)-6-oxohexahydropyrrolo[3,2-b]pyrrole-1(2H)-carboxamide;
(3aR,6aS)-4-Benzoyl-N-(6-chloro-2-fluoro-3-methylbenzyl)-6-oxohexahydropyrrolo[3,2-b]pyrrole-1(2H)-carboxamide;
(3aR,6aS)—N-(6-Chloro-2-fluoro-3-methylbenzyl)-6-oxo-4-(pyrrolidine-1-carbonyl)hexahydropyrrolo[3,2-b]pyrrole-1(2H)-carboxamide;
(3aR,6aS)-4-Benzoyl-N-(biphenyl-2-yl)-6-oxohexahydropyrrolo[3,2-b]pyrrole-1(2H)-carboxamide;
(3aR,6aS)-4-Benzoyl-N-(2-ethoxyphenyl)-6-oxohexahydropyrrolo[3,2-b]pyrrole-1(2H)-carboxamide;
(3aR,6aS)-4-Benzoyl-6-oxo N-(2-propylphenyl)hexahydropyrrolo[3,2-b]pyrrole-1(2H)-carboxamide;
(3aR,6aS)-4-Benzoyl-N-(2-chloro-5-(trifluoromethyl)phenyl)-6-oxohexahydropyrrolo[3,2-b]pyrrole-1(2H)-carboxamide;
(3aS,6aR)-3-Oxo-N-(1-(thiophen-3-yl)butan-2-yl)tetrahydro-2H-furo[3,2-b]pyrrole-4(5H)-carboxamide;
(3aS,6aR)-3-Oxo-N-(1-(thiophen-2-yl)butan-2-yl)tetrahydro-2H-furo[3,2-b]pyrrole-4(5H)-carboxamide;
(3aS,6aR)-3-Oxo-N-((1-(phenoxymethyl)cyclobutyl)methyl)tetrahydro-2H-furo[3,2-b]pyrrole-4(5H)-carboxamide;
(3aS,6aR)—N-(5-Methyl-1-(thiophen-2-yl)hexan-3-yl)-3-oxotetrahydro-2H-furo[3,2-b]pyrrole-4(5H)-carboxamide;
(3aS,6aR)—N-(6-Chloro-2-fluoro-3-methylbenzyl)-3-oxotetrahydro-2H-furo[3,2-b]pyrrole-4(5H)-carboxamide;
(3aS,6aR)—N-(Biphenyl-2-yl)-3-oxotetrahydro-2H-furo[3,2-b]pyrrole-4(5H carboxamide;
(3aS,6aR)—N-(2-Ethoxyphenyl)-3-oxotetrahydro-2H-furo[3,2-b]pyrrole-4(5H)-carboxamide;
(3aS,6&R)-3-Oxo-N-(2-propylphenyl)tetrahydro-2H-furo[3,2-b]pyrrole-4(5H)-carboxamide;
(3aS,6aR)—N-(2-Chloro-5-(trifluoromethyl)phenyl)-3-oxotetrahydro-2H-furo[3,2-b]pyrrole-4(5H)-carboxamide;
(3aR,6aS)—S-6-Chloro-2-fluoro-3-methylbenzyl 4-benzoyl-6-oxohexahydropyrrolo[3,2-b]pyrrole-1(2H)-carbothioate;
(3R,3aR,6aR)—S-6-chloro-2-fluoro-3-methylbenzyl 3-hydroxytetrahydro-2H-furo[3,2-b]pyrrole-4(5H)-carbothioate;
(3aR,6aS)-2-Ethoxy-4-methylphenyl 4-benzoyl-6-oxohexahydropyrrolo[3,2-b]pyrrole-1(2H)-carboxylate;
(3aR,6aS)-2-Isopropoxyphenyl 4-benzoyl-6-oxohexahydropyrrolo[3,2-b]pyrrole-1(2H)-carboxylate;
(3aR,6aS)-2-Propylphenyl 4-benzoyl-6-oxohexahydropyrrolo[3,2-b]pyrrole-1(2H)-carboxylate;
(3aR,6aS)-(2-Methyl-6-(trifluoromethyl)pyridin-3-yl)methyl 4-benzoyl-6-oxohexahydropyrrolo[3,2-b]pyrrole-1(2H)-carboxylate;
(3aR,6aS)-2-Fluoro-6-(trifluoromethyl)benzyl 4-benzoyl-6-oxohexahydropyrrolo[3,2-b]pyrrole-1(2H)-carboxylate;
(3aR,6aS)-6-Chloro-2-fluoro-3-methylbenzyl 4-benzoyl-6-oxohexahydropyrrolo[3,2-b]pyrrole-1(2H)-carboxylate;
(3aS,6aR)-2-Propylphenyl 3-oxotetrahydro-2H-furo[3,2-b]pyrrole-4(5H)-carboxylate;
   and pharmaceutically acceptable salts thereof.

### Pharmaceutical Compositions

A further aspect of the invention relates to a pharmaceutical composition comprising a compound of the invention admixed with one or more pharmaceutically acceptable diluents, excipients or carriers. Other active materials may also be present, as may be considered appropriate or advisable for the disease or condition being treated or prevented.

Even though the compounds of the present invention (including their pharmaceutically acceptable salts, esters and pharmaceutically acceptable solvates) can be administered alone, they will generally be administered in admixture with a pharmaceutical carrier, excipient or diluent, particularly for human therapy. The pharmaceutical compositions may be for human or animal usage in human and veterinary medicine.

Examples of such suitable excipients for the various different forms of pharmaceutical compositions described herein may be found in the “Handbook of Pharmaceutical Excipients, 2^{nd} Edition, (1994), Edited by A Wade and P J Weller. The carrier, or, if more than one be present, each of the carriers, must be acceptable in the sense of being compatible with the other ingredients of the formulation and not deleterious to the recipient.

Acceptable carriers or diluents for therapeutic use are well known in the pharmaceutical art, and are described, for example, in Remington's Pharmaceutical Sciences, Mack Publishing Co. (A. R. Gennaro edit. 1985).

Examples of suitable carriers include lactose, starch, glucose, methyl cellulose, magnesium stearate, mannitol, sorbitol and the like. Examples of suitable diluents include ethanol, glycerol and water.

The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), solubilising agent(s).

Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol.

Examples of suitable lubricants include sodium oleate, sodium stearate, magnesium stearate, sodium benzoate, sodium acetate, sodium chloride and the like.

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

According to a further aspect of the invention, there is provided a process for the preparation of a pharmaceutical or veterinary composition as described above, the process comprising bringing the active compound(s) into association with the carrier, for example by admixture.

In general, the formulations are prepared by uniformly and intimately bringing into association the active agent with liquid carriers or finely divided solid carriers or both, and then if necessary shaping the product. The invention extends to methods for preparing a pharmaceutical composition comprising bringing a compound of general formula (I) in conjunction or association with a pharmaceutically or veterinarily acceptable carrier or vehicle.

### Salts/Esters

The compounds of the invention can be present as salts or esters, in particular pharmaceutically and veterinarily acceptable salts or esters.

Pharmaceutically acceptable salts of the compounds of the invention include suitable acid addition or base salts thereof. A review of suitable pharmaceutical salts may be found in Berge et al, J Pharm Sci, 66, 1-19 (1977). Salts are formed, for example with strong inorganic acids such as mineral acids, e.g. hydrohalic acids such as hydrochloride, hydrobromide and hydroiodide, sulphuric acid, phosphoric acid sulphate, bisulphate, hemisulphate, thiocyanate, persulphate and sulphonic acids; with strong organic carboxylic acids, such as alkanecarboxylic acids of 1 to 4 carbon atoms which are unsubstituted or substituted (e.g., by halogen), such as acetic acid; with saturated or unsaturated dicarboxylic acids, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid. Salts which are not pharmaceutically or veterinarily acceptable may still be valuable as intermediates.

Preferred salts include, for example, acetate, trifluoroacetate, lactate, gluconate, citrate, tartrate, maleate, malate, pantothenate, adipate, alginate, aspartate, benzoate, butyrate, digluconate, cyclopentanate, glucoheptanate, glycerophosphate, oxalate, heptanoate, hexanoate, fumarate, nicotinate, palmoate, pectinate, 3-phenylpropionate, picrate, pivalate, proprionate, tartrate, lactobionate, pivolate, camphorate, undecanoate and succinate, organic sulphonic acids such as methanesulphonate, ethanesulphonate, 2-hydroxyethane sulphonate, camphorsulphonate, 2-naphthalenesulphonate, benzenesulphonate, p-chlorobenzenesulphonate and p-toluenesulphonate; and inorganic acids such as hydrochloride, hydrobromide, hydroiodide, sulphate, bisulphate, hemisulphate, thiocyanate, persulphate, phosphoric and sulphonic acids.

Esters are formed either using organic acids or alcohols/hydroxides, depending on the functional group being esterified. Organic acids include carboxylic acids, such as alkanecarboxylic acids of 1 to 12 carbon atoms which are unsubstituted or substituted (e.g., by halogen), such as acetic acid; with saturated or unsaturated dicarboxylic acid, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid. Suitable hydroxides include inorganic hydroxides, such as sodium hydroxide, potassium hydroxide, calcium hydroxide, aluminium hydroxide. Alcohols include alkanealcohols of 1-12 carbon atoms which may be unsubstituted or substituted, e.g. by a halogen).

### Enantiomers/Tautomers

In all aspects of the present invention previously discussed, the invention includes, where appropriate all enantiomers, diastereoisomers and tautomers of the compounds of the invention. The person skilled in the art will recognise compounds that possess optical properties (one or more chiral carbon atoms) or tautomeric characteristics. The corresponding enantiomers and/or tautomers may be isolated/prepared by methods known in the art.

Compounds of the invention containing a chiral centre may be used as a racemic mixture, an enantiomerically enriched mixture, or the racemic mixture may be separated using well-known techniques and an individual enantiomer may be used alone.

### Stereo and Geometric Isomers

Some of the compounds of the invention may exist as stereoisomers and/or geometric isomers—e.g. they may possess one or more asymmetric and/or geometric centres and so may exist in two or more stereoisomeric and/or geometric forms. The present invention contemplates the use of all the individual stereoisomers and geometric isomers of those inhibitor agents, and mixtures thereof. The terms used in the claims encompass these forms, provided said forms retain the appropriate functional activity (though not necessarily to the same degree).

The present invention also includes all suitable isotopic variations of the agent or a pharmaceutically acceptable salt thereof. An isotopic variation of an agent of the present invention or a pharmaceutically acceptable salt thereof is defined as one in which at least one atom is replaced by an atom having the same atomic number but an atomic mass different from the atomic mass usually found in nature. Examples of isotopes that can be incorporated into the agent and pharmaceutically acceptable salts thereof include isotopes of hydrogen, carbon, nitrogen, oxygen, phosphorus, sulphur, fluorine and chlorine such as 2K, ³H, ¹³C, ¹⁴C, ¹⁵N, ¹⁷O, ¹⁸O, ³¹P, ³²P, ³⁵S, ¹⁸F and ³⁶Cl, respectively. Certain isotopic variations of the agent and pharmaceutically acceptable salts thereof, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the agent of the present invention and pharmaceutically acceptable salts thereof of this invention can generally be prepared by conventional procedures using appropriate isotopic variations of suitable reagents.

### Prodrugs

The invention further includes the compounds of the present invention in prodrug form, i.e. covalently bonded compounds which release the active parent drug according to general formula (I) in vivo. Such prodrugs are generally compounds of the invention wherein one or more appropriate groups have been modified such that the modification may be reversed upon administration to a human or mammalian subject Reversion is usually performed by an enzyme naturally present in such subject, though it is possible for a second agent to be administered together with such a prodrug in order to perform the reversion in vivo Examples of such modifications include ester (for example, any of those described above), wherein the reversion may be carried out be an esterase etc. Other such systems will be well known to those skilled in the art.

A prodrug may for example constitute a ketal or hemiketal derivative of the exocyclic ketone functionality present in the tetrahydro-furo[3,2-b]pyrrol-3-one or tetrahydro-furo[3,2-c]isoxazol-6-one or tetrahydro-furo[3,2-c]pyrazol-6-one or hexahydro-pyrrolo[3,2-b]pyrrol-3-one or hexahydro-2-oxa-1,4-diaza-pentalen-6-one or hexahydro-pyrrolo[3,2-c]pyrazol-6-one scaffold.

### Solvates

The present invention also includes solvate forms of the compounds of the present invention. The terms used in the claims encompass these forms.

### Polymorphs

The invention further relates to the compounds of the present invention in their various crystalline forms, polymorphic forms and (an)hydrous forms. It is well established within the pharmaceutical industry that chemical compounds may be isolated in any of such forms by slightly varying the method of purification and or isolation form the solvents used in the synthetic preparation of such compounds.

If different structural isomers are present, and/or one or more chiral centres are present, all isomeric forms are intended to be covered. Enantiomers are characterised by the absolute configuration of their chiral centres and described by the R- and S-sequencing rules of Cahn, Ingold and Prelog. Such conventions are well known in the art (e.g. see ‘Advanced Organic Chemistry’, 3^{rd} edition, ed. March, J., John Wiley and Sons, New York, 1985). It is also intended to include compounds of general formula (a) where any hydrogen atom has been replaced by a deuterium atom.

### Assays

Another aspect of the invention relates to the use of a compound of the invention as defined hereinabove in an assay for identifying further candidate compounds that influence the activity of one or cysteine proteinases.

Preferably, the assay is capable of identifying candidate compounds that are capable of inhibiting one or more CAC1 cysteine proteinases.

More preferably, the assay is a competitive binding assay.

Preferably, the candidate compound is generated by conventional SAR modification of a compound of the invention.

As used herein, the term “conventional SAR modification” refers to standard methods known in the art for varying a given compound by way of chemical derivatisation.

Thus, in one aspect, the identified compound may act as a model (for example, a template) for the development of other compounds. The compounds employed in such a test may be free in solution, affixed to a solid support, borne on a cell surface, or located intracellularly. The abolition of activity or the formation of binding complexes between the compound and the agent being tested may be measured.

The assay of the present invention may be a screen, whereby a number of agents are tested. In one aspect, the assay method of the present invention is a high through-put screen.

This invention also contemplates the use of competitive drug screening assays in which neutralising antibodies capable of binding a compound specifically compete with a test compound for binding to a compound.

Another technique for screening provides for high throughput screening (HTS) of agents having suitable binding affinity to the substances and is based upon the method described in detail in WO 84/03564.

It is expected that the assay methods of the present invention will be suitable for both small and large-scale screening of test compounds as well as in quantitative assays.

Preferably, the competitive binding assay comprises contacting a compound of the invention with a cysteine proteinase in the presence of a known substrate of said enzyme and detecting any change in the interaction between said cysteine proteinase and said known substrate.

A further aspect of the invention provides a method of detecting the binding of a ligand to a cysteine proteinase, said method comprising the steps of:
(i) contacting a ligand with cysteine proteinase in the presence of a known substrate of said enzyme;
(ii) detecting any change in the interaction between said enzyme and said known substrate;
   and wherein said ligand is a compound of the invention.

One aspect of the invention relates to a process comprising the steps of:
(a) performing an assay method described hereinabove;
(b) identifying one or more ligands capable of binding to a ligand binding domain; and
(c) preparing a quantity of said one or more ligands.

Another aspect of the invention provides a process comprising the steps of:
(a) performing an assay method described hereinabove;
(b) identifying one or more ligands capable of binding to a ligand binding domain; and
(c) preparing a pharmaceutical composition comprising said one or more ligands.

Another aspect of the invention provides a process comprising the steps of:
(a) performing an assay method described hereinabove;
(b) identifying one or more ligands capable of binding to a ligand binding domain;
(c) modifying said one or more ligands capable of binding to a ligand binding domain;
(d) performing the assay method described hereinabove;
(e) optionally preparing a pharmaceutical composition comprising said one or more ligands.

The invention also relates to a ligand identified by the method described hereinabove.

Yet another aspect of the invention relates to a pharmaceutical composition comprising a ligand identified by the method described hereinabove.

Another aspect of the invention relates to the use of a ligand identified by the method described hereinabove in the preparation of a pharmaceutical composition for use in the treatment of one or more disorders selected from osteoporosis, Paget's disease, Chagas's disease, malaria, gingival disease such as gingivitis or periodontitis, hypercalaemia, metabolic bone disease and diseases involving matrix or cartilage degradation, such as osteoarthritis, rheumatoid arthritis and neoplastic diseases.

The above methods may be used to screen for a ligand useful as an inhibitor of one or more cysteine proteinases.

Compounds of general formula (I) are useful both as laboratory tools and as therapeutic agents. In the laboratory certain compounds of the invention are useful in establishing whether a known or newly discovered cysteine proteinase contributes a critical or at least significant biochemical function during the establishment or progression of a disease state, a process commonly referred to as ‘target validation’.

According to a further aspect of the invention, there is provided a method of validating a known or putative cysteine proteinase as a therapeutic target, the method comprising:

(a) assessing the in vitro binding of a compound as described above to an isolated known or putative cysteine proteinase, providing a measure of potency; and optionally, one or more of the steps of:
(b) assessing the binding of the compound to closely related homologous proteinases of the target and general house-keeping proteinases (e.g. trypsin) to provides a measure of selectivity;
(c) monitoring a cell-based functional marker of a particular cysteine proteinase activity, in the presence of the compound; and
(d) monitoring an animal model-based functional marker of a particular cysteine proteinase activity in the presence of the compound.

The invention therefore provides a method of validating a known or putative cysteine proteinase as a therapeutic target. Differing approaches and levels of complexity are appropriate to the effective inhibition and ‘validation’ of a particular target. In the first instance, the method comprises assessing the in vitro binding of a compound of general formula (I) to an isolated known or putative cysteine proteinase, providing a measure of ‘potency’. An additional assessment of the binding of a compound of general formula (I) to closely related homologous proteinases of the target and general house-keeping proteinases (e.g. trypsin) provides a measure of ‘selectivity’. A second level of complexity may be assessed by monitoring a cell-based functional marker of a particular cysteine proteinase activity, in the presence of a compound of general formula (I). For example, an ‘osteoclast resorption assay’ has been utilised as a cell-based secondary in vitro testing system for monitoring the activity of cathepsin K and the biochemical effect of proteinase inhibitors (e.g. see WO-A-9850533). An ‘MHC-II processing—T-cell activation assay’ has been utilised as a cell-based secondary in vitro testing system for monitoring the activity of cathepsin S and the biochemical effect of proteinase inhibitors (Shi, G-P., et al, Immunity, 10, 197-206, 1999). When investigating viral or bacterial infections such a marker could simply be a functional assessment of viral (e.g. count of mRNA copies) or bacterial loading and assessing the biochemical effect of proteinase inhibitors. A third level of complexity may be assessed by monitoring an animal model-based functional marker of a particular cysteine proteinase activity, in the presence of a compound of general formula (I). For example, murine models of Leishmania infection, P. vinckei infection, malaria (inhibition of falcipain) and T. cruzi infection (cruzipain), indicate that inhibition of cysteine proteinases that play a key role in pathogen propagation is effective in arresting disease symptoms, ‘validating’ said targets.

The invention therefore extends to the use of a compound of general formula (I) in the validation of a known or putative cysteine proteinase as a therapeutic target.

### Therapeutic Use

Compounds of general formula (I) are useful for the in vivo treatment or prevention of diseases in which participation of a cysteine proteinase is implicated in particular, compounds of general formula I are inhibitors of a wide range of CAC1 cysteinyl proteinases for example cathepsin K, cathepsin S, cathepsin L, cathepsin F, cathepsin B, cathepsin V, cruzipains, falcipains and leismania mexzcana CPB proteinase.

According to a further aspect of the invention, there is provided a compound of general formula (a) for use in medicine, especially for preventing or treating diseases in which the disease pathology may be modified by inhibiting a cysteine proteinase.

According to a further aspect of the invention, there is provided the use of a compound of general formula (I) in the preparation of a medicament for preventing or treating diseases in which the disease pathology may be modified by inhibiting a cysteine proteinase.

Certain cysteine proteinases function in the normal physiological process of protein degradation in animals, including humans, e.g. in the degradation of connective tissue. However, elevated levels of these enzymes in the body can result in pathological conditions leading to disease. Thus, cysteine proteinases have been implicated in various disease states, including but not limited to, infections by Pneumocystis carinii, Trypsanoma cruzi, Trypsanoma brucei brucel and Crithidia fusiculata; as well as in osteoporosis, osteoarthritis, rheumatoid arthritis, multiple sclerosis, chronic pain, autoimmunity, schistosomiasis, malaria, tumour metasasis, metachromatic leukodystrophy, muscular dystrophy, amytrophy, and the like (see WO-A-9404172 and EP-A-0603873 and references cited therein). Additionally, a secreted bacterial cysteine proteinase from S. Aureus called staphylopain has been implicated as a bacterial virulence factor (Potempa, J., et al. J. Biol. Chem., 262(6), 2664-2667, 1998).

The invention is useful in the prevention and/or treatment of each of the disease states mentioned or implied above. The present invention also is useful in a methods of treatment or prevention of diseases caused by pathological levels of cysteine proteinases, particularly cysteine proteinases of the papain superfamily, which methods comprise administering to an animal, particularly a mammal, most particularly a human, in need thereof a compound of the present invention. The present invention particularly provides methods for treating diseases in which cysteine proteinases are implicated, including infections by Pneumocystis carinii, Trypsanoma cruzi, Trypsanoma brucei, Leishmania mexicana, Clostridium histolyticum, Staphylococcus aureus, foot-and-mouth disease virus and Crithidia fusiculata; as well as in osteoporosis, osteoarthritis, rheumatoid arthritis, multiple sclerosis, chronic pain, autoimmunity, schistosomiasis, malaria, tumour metastasis, metachromatic leukodystrophy, muscular dystrophy, amytrophy.

Inhibitors of cathepsin K, particularly cathepsin K-specific compounds, are useful for the treatment of osteoporosis, Paget's disease, gingival diseases such as gingivitis and periodontitis, hypercalaemia of malignancy, metabolic bone disease, diseases involving matrix or cartilage degradation, in particular osteoarthritis and rheumatoid arthritis and neoplastic diseases.

Preferred features for each aspect of the invention are as for each other aspect mutatis mutandis.

### Administration

The pharmaceutical compositions of the present invention may be adapted for rectal, nasal, intrabronchial, topical (including buccal and sublingual), vaginal or parenteral (including subcutaneous, intramuscular, intravenous, intraarterial and intradermal), intraperitoneal or intrathecal administration. Preferably the formulation is an orally administered formulation. The formulations may conveniently be presented in unit dosage form, i.e., in the form of discrete portions containing a unit dose, or a multiple or sub-unit of a unit dose. By way of example, the formulations may be in the form of tablets and sustained release capsules, and may be prepared by any method well known in the art of pharmacy.

Formulations for oral administration in the present invention may be presented as: discrete units such as capsules, gellules, drops, cachets, pills or tablets each containing a predetermined amount of the active agent; as a powder or granules; as a solution, emulsion or a suspension of the active agent in an aqueous liquid or a non-aqueous liquid; or as an oil-in-water liquid emulsion or a water-in-oil liquid emulsion; or as a bolus etc. Preferably, these compositions contain from 1 to 250 mg and more preferably from 10-100 mg, of active ingredient per dose.

For compositions for oral administration (e.g. tablets and capsules), the term “acceptable carrier” includes vehicles such as common excipients e.g. binding agents, for example syrup, acacia, gelatin, sorbitol, tragacanth, polyvinylpyrrolidone (Povidone), methylcellulose, ethylcellulose, sodium carboxymethylcellulose, hydroxypropylmethylcellulose, sucrose and starch; fillers and carriers, for example corn starch, gelatin, lactose, sucrose, microcrystalline cellulose, kaolin, mannitol, dicalcium phosphate, sodium chloride and alginic acid; and lubricants such as magnesium stearate, sodium stearate and other metallic stearates, glycerol stearate stearic acid, silicone fluid, talc waxes, oils and colloidal silica Flavouring agents such as peppermint, oil of wintergreen, cherry flavouring and the like can also be used. It may be desirable to add a colouring agent to make the dosage form readily identifiable. Tablets may also be coated by methods well known in the art

A tablet may be made by compression or moulding, optionally with one or more accessory ingredients. Compressed tablets may be prepared by compressing in a suitable machine the active agent in a free flowing form such as a powder or granules, optionally mixed with a binder, lubricant, inert diluent, preservative, surface-active or dispersing agent. Moulded tablets may be made by moulding in a suitable machine a mixture of the powdered compound moistened with an inert liquid diluent. The tablets may be optionally be coated or scored and may be formulated so as to provide slow or controlled release of the active agent.

Other formulations suitable for oral administration include lozenges comprising the active agent in a flavoured base, usually sucrose and acacia or tragacanth; pastilles comprising the active agent in an inert base such as gelatin and glycerin, or sucrose and acacia; and mouthwashes comprising the active agent in a suitable liquid carrier.

Other forms of administration comprise solutions or emulsions which may be injected intravenously, intraarterially, intrathecally, subcutaneously, intradermally, intraperitoneally or intramuscularly, and which are prepared from sterile or sterilisable solutions. Injectable forms typically contain between 10-1000 mg, preferably between 10-250 mg, of active ingredient per dose.

The pharmaceutical compositions of the present invention may also be in form of suppositories, pessaries, suspensions, emulsions, lotions, ointments, crearns, gels, sprays, solutions or dusting powders.

An alternative means of transdermal administration is by use of a skin patch. For example, the active ingredient can be incorporated into a cream consisting of an aqueous emulsion of polyethylene glycols or liquid paraffin. The active ingredient can also be incorporated; at a concentration of between 1 and 10% by weight, into an ointment consisting of a white wax or white soft paraffin base together with such stabilisers and preservatives as may be required.

### Dosage

A person of ordinary skill in the art can easily determine an appropriate dose of one of the instant compositions to administer to a subject without undue experimentation. Typically, a physician will determine the actual dosage which will be most suitable for an individual patient and it will depend on a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. The dosages disclosed herein are exemplary of the average case. There can of course be individual instances where higher or lower dosage ranges are merited, and such are within the scope of this invention.

In accordance with this invention, an effective amount of a compound of general formula (I) may be administered to inhibit the proteinase implicated with a particular condition or disease. Of course, this dosage amount will further be modified according to the type of administration of the compound. For example, to achieve an “effective amount” for acute therapy, parenteral administration of a compound of general formula (I) is preferred. An intravenous infusion of the compound in 5% dextrose in water or normal saline, or a similar formulation with suitable excipients, is most effective, although an intramuscular bolus injection is also useful. Typically, the parenteral dose will be about 0.01 to about 100 mg/kg; preferably between 0.1 and 20 mg/kg, in a manner to maintain the concentration of drug in the plasma at a concentration effective to inhibit a cysteine proteinase. The compounds may be administered one to four times daily at a level to achieve a total daily dose of about 0.4 to about 400 mg/kg/day. The precise amount of an inventive compound which is therapeutically effective, and the route by which such compound is best administered, is readily determined by one of ordinary skill in the art by comparing the blood level of the agent to the concentration required to have a therapeutic effect. Prodrugs of compounds of the present invention may be prepared by any suitable method. For those compounds in which the prodrug moiety is a ketone functionality, specifically ketals and/or hemiketals, the conversion may be effected in accordance with conventional methods.

The compounds of this invention may also be administered orally to the patient, in a manner such that the concentration of drug is sufficient to inhibit bone resorption or to achieve any other therapeutic indication as disclosed herein. Typically, a pharmaceutical composition containing the compound is administered at an oral dose of between about 0.1 to about 50 mg/kg in a manner consistent with the condition of the patient. Preferably the oral dose would be about 0.5 to about 20 mg/kg.

No unacceptable toxicological effects are expected when compounds of the present invention are administered in accordance with the present invention. The compounds of this invention, which may have good bioavailability, may be tested in one of several biological assays to determine the concentration of a compound which is required to have a given pharmacological effect.

### Combinations

In a particularly preferred embodiment, the one or more compounds of the invention are administered in combination with one or more other active agents, for example, existing drugs available on the market. In such cases, the compounds of the invention may be administered consecutively, simultaneously or sequentially with the one or more other active agents.

Drugs in general are more effective when used in combination. In particular, combination therapy is desirable in order to avoid an overlap of major toxicities, mechanism of action and resistance mechanism(s). Furthermore, it is also desirable to administer most drugs at their maximum tolerated doses with minimum time intervals between such doses. The major advantages of combining chemotherapeutic drugs are that it may promote additive or possible synergistic effects through biochemical interactions and also may decrease the emergence of resistance in early tumor cells which would have been otherwise responsive to initial chemotherapy with a single agent. An example of the use of biochemical interactions in selecting drug combinations is demonstrated by the administration of leucovorin to increase the binding of an active intracellular metabolite of 5-fluorouracil to its target, thymidylate synthase, thus increasing its cytotoxic effects.

Beneficial combinations may be suggested by studying the inhibitory activity of the test compounds with agents known or suspected of being valuable in the treatment of a particular disorder. This procedure can also be used to determine the order of administration of the agents, i.e. before, simultaneously, or after delivery. Such scheduling may be a feature of all the active agents identified herein.

### Synthesis

To those skilled in the practices of organic chemistry, compounds of general formula (I) may be readily synthesised by a number of chemical strategies, performed either in solution or on the solid phase (see Atherton, E. and Sheppard, R. C. In ‘Solid Phase Peptide Synthesis: A Practical Approach’, Oxford University Press, Oxford, U.K. 1989, for a general review of solid phase synthesis principles). The solid phase strategy is attractive in being able to generate many thousands of analogues, typically on a 5-100 mg scale, through established parallel synthesis methodologies (e.g. see (a) Bastos, M.; Maeji, N. J.; Abeles, R. H. Proc. Natl. Acad. Sci. USA, 92, 6738-6742, 1995). The solution phase strategy is attractive in being able to generate larger quantities of preferred analogues, typically on a multi-gram to multi-kilogram scale.

Another aspect of the invention therefore relates to a process of preparing a compound of formula I as defined in claim 1, said process comprising the step of converting a compound of formula II to a compound of formula I,

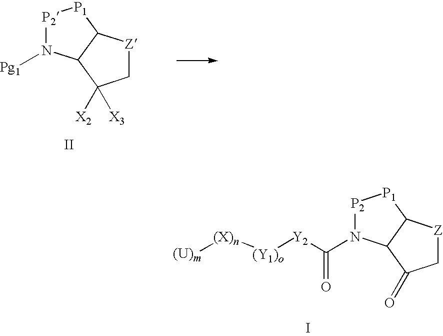

wherein

### P₂′ is O,

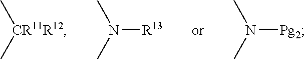

### Z′ is O,

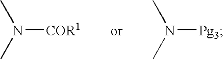

X₂ and X₃ together form ═O, or are each independently OR, where R′ is H or alkyl;
Pg₁, Pg₂ and Pg₃ are each independently amine protecting groups; and
P₁, P₂, Z, Y₂, Y₁, X, U, R¹, R¹¹⁻¹³, m, n and o are as defined above.

In one preferred embodiment, Pg₁, Pg₂ and Pg₃ are each independently selected from 9-fluorenylmethoxycarbonyl (Fmoc), tert-butoxycarbonyl (Boc), benzyloxycarbonyl (Cbz), allyloxycarbonyl (Alloc) and trichloroethoxycarbonyl (Treoc).

Preferably, the process of the invention comprises the steps of:
(i) converting a compound of formula III to a compound of formula IV;
(ii) attaching said compound of formula IV to a solid phase resin via a linker to form an intermediate species of formula V;
(iii) removing protecting group Pg₁ from said intermediate species of formula V and converting to an intermediate species of formula VI; and
(iv) removing said compound of formula I from the solid phase resin

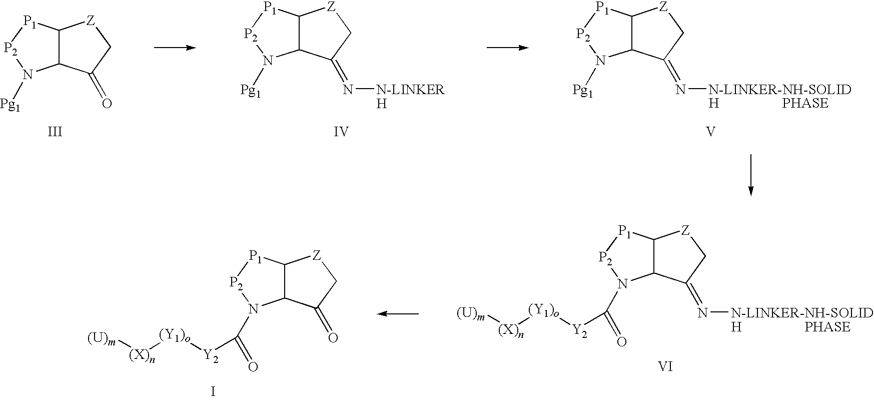

In one particularly preferred embodiment of the invention, Pg₁ is Fmoc.

In one preferred embodiment of the invention, the process comprises removing protecting group Pg₁ and reacting the intermediate so produced with a compound selected from:

(U)ₘ(X)ₙ(Y₁)ₒ—O(CO)Cl;

(U)ₘ(X)ₙ(Y₁)ₒ—S(CO)Cl;

(U)ₘ(X)ₙ(Y₁)ₒ—N═C═O; and

(U)ₘ(X)ₙ(Y₁)ₒ—NH(CO)Cl.

In one preferred embodiment of the invention, Z′ is

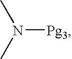

and said process further comprises the step of removing said Pg₃ group and reacting the compound so produced with a compound selected from:
R¹COOH;
R²SO₂Cl;
R¹N═C═O;
R¹OCOCl; and
R³COCl;
   where R¹, R² and R³ are as defined above.

In one particularly preferred embodiment of the invention, the process comprises attaching a compound of formula (15) to a solid phase resin to form an intermediate species of formula (16), and subsequently converting to a species of formula (17)

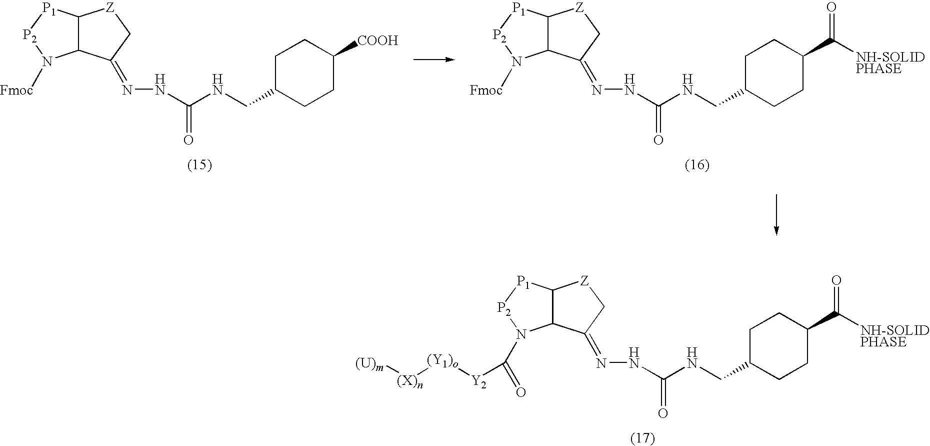

In one preferred embodiment of the invention, the process comprises the step of:

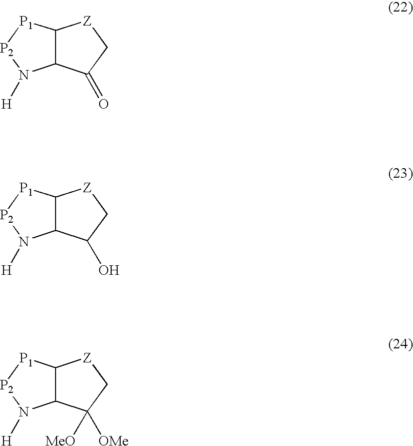

reacting a compound of formula (22), (23) or (24), where Z is O,

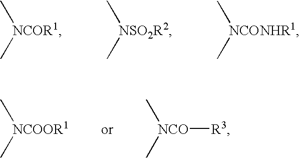

with a compound selected from:

(U)ₘ(X)ₙ(Y₁)ₒ—O(CO)Cl;

(U)ₘ(X)ₙ(Y₁)ₒ—S(CO)Cl;

(U)ₘ(X)ₙ(Y₁)ₒ—N—C═O; and

(U)ₘ(X)ₙ(Y₁)ₒ—NH(CO)Cl;

where P₁, P₂, U, X, Y₁, m, n and o are as defined above.

In one preferred embodiment of the invention, the process comprises the steps of:

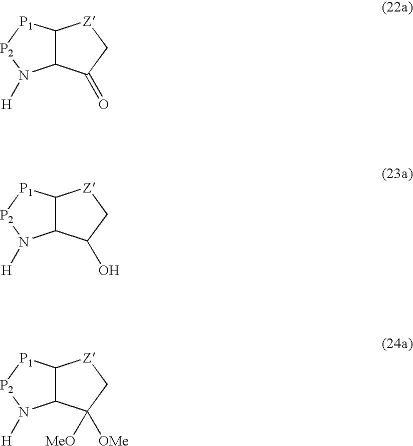

reacting a compound of formula (22a), (23a) or (24a), where Z′ is

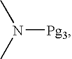

with a compound selected from:

(U)ₘ(X)ₙ(Y₁)ₒ—O(CO)Cl;

(U)ₘ(X)ₙ(Y₁)ₒ—S(CO)Cl;

(U)ₘ(X)ₙ(Y₁)ₒ—N═C═O; and

(U)ₘ(X)ₙ(Y₁)ₒ—NH(CO)Cl;

where P₁, P₂, U, X, Y₁, m, n and o are as defined above; and
converting said

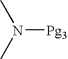

group to a group selected from

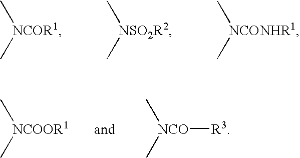

In more detail, one strategy for the synthesis of compounds of general formula (I) comprises:—
(a) Preparation of an appropriately functionalised and protected bicyclic ketone or bicyclic alcohol building block in solution;
(b) Attachment of the building block (a) to the solid phase through a linker that is stable to the conditions of synthesis, but readily labile to cleavage at the end of a synthesis (see James, I. W., Tetrahedron, 55(Report No 489), 4855-4946, 1999, for examples of the ‘linker’ function as applied to solid phase synthesis);
(c) Solid phase organic chemistry (see Brown, P D. J. Chem. Soc., Perkin Trans. 1, 19, 3293-3320, 1998), to construct the remainder of the molecule;
(d) Compound cleavage from the solid phase into solution; and
(e) Cleavage work-up and compound analysis.

The first stage in a synthesis of compounds of general formula (I) is the preparation in solution of a functionalised and protected building block. Synthesis of the protected tetrahydro-furo[3,2-b]pyrrol-3-ones such as (9a) has been previously detailed (see Quibell, M. et. al., Bioorg. Med. Chem. 12, 5689-5710, 2004). The corresponding protected tetrahydro-furo[3,2-c]isoxazol-6-ones such as (10a) and protected tetrahydro-furo[3,2-c]pyrazol-6-ones such as (11a) may be accessed following the general schemes detailed in Quibell, M. et. al., Bioorg. Med. Chem. 12, 5689-5710, 2004, through the use of the analogous 5-oxo-proline and 5-aza-proline starting acids.

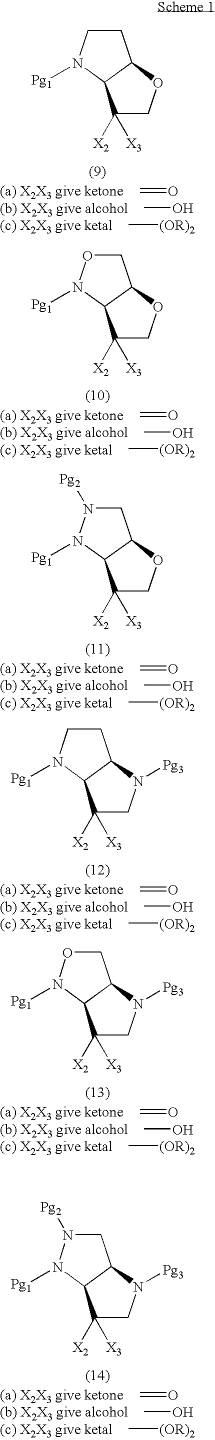

Synthesis of the protected hexahydro-pyrrolo[3,2-b]pyrrol-3-ones such as (12a) has been previously detailed (see Quibell, M. et. al., Bioorg. Med. Chem. 1, 609-625, 2005) whilst protected hexahydro-pyrrolo[3,2-c]pyrazol-6-ones such as (13a) and protected hexahydro-2-oxa-1,4-diaza-pentalen-6-ones such as (14a) have been detailed (see Wang, Y. et. al., Bioorg. Med. Chem. Lett. A, 1327-1331, 2005). The analogous bicyclic alcohol intermediates (9b-14b) are also readily available (see (i) Quibell, M et. al., Bioorg. Med. Chem. 1, 609-625, 2005. (ii) Wang, Y. et. al., Bioorg. Med. Chem. Lett. X, 1327-1331, 2005. (Hii) WO-A-02057270).

‘Pg₁’, ‘Pg₂’ and ‘Pg₃’ denote suitable amine protecting groups which include but are not limited to the 9-fluorenylmethoxycarbonyl (Fmoc, see Atherton, E. and Sheppard, R. C. In ‘Solid Phase Peptide Sythesis: A Practical Approach’, Oxford University Press, Oxford, U.K. 1989), tert-butoxycarbonyl (Boc), benzyloxycarbonyl (Cbz), allyloxycarbonyl (Alloc) and trichloroethoxycarbonyl (Treoc) for example. Alternatively, any ‘Pg₁’, ‘Pg₂’ or ‘Pg₃’ may also denote a substituent group covalently bonded to the nitrogen e.g. ‘Pg₃’ may be the final —C(O)R¹ acyl group such as benzoyl or the final —SO₂R² sulphonyl group such as phenylsulphonyl; ‘Pg₂’ may be the final -R¹³ group; ‘Pg₁’ may be the (U)ₘ—(X)ₙY₁)ₒ—Y₂ group.

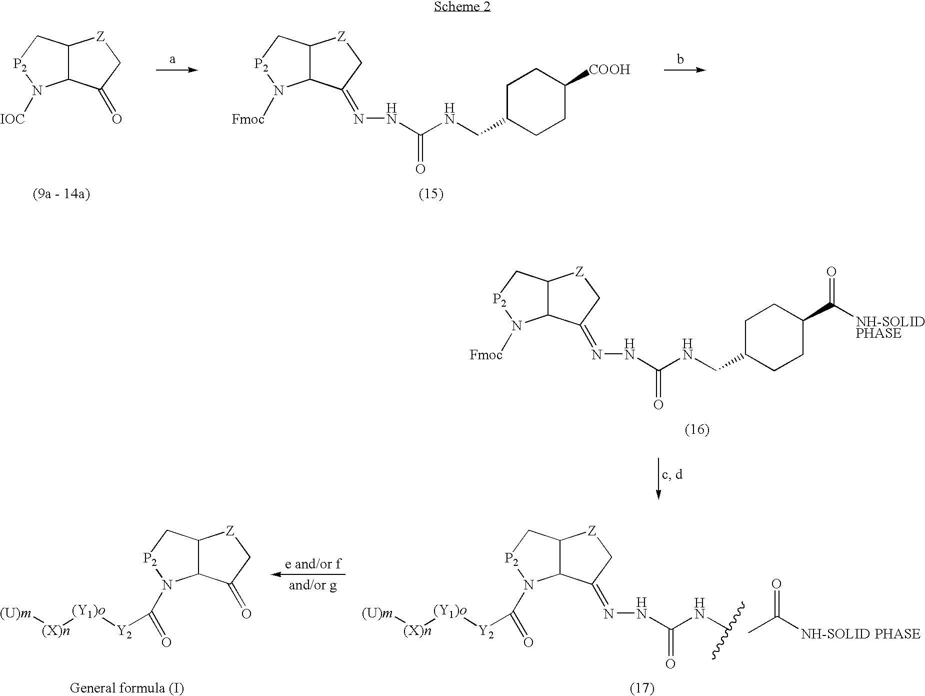

Scheme 2. (a) (9a-14a) in 90% EtOH/H₂O/NaOAc/4-[[(hydrazinocarbonyl)amino]methyl]-cyclohexane carboxylic acid.trifluoroacetate, reflux. (b) 3 eq construct (15)/3 eq HBTU/3 eq HOBt/6 eq NMM, NH₂-SOLID PHASE, DMF, RT, o/nl (c) 20% piperidine/DMF, RT, 30 min. (d) Range of chemistries to couple U—X—Y₁—Y₂. (e) Where Z is O and P₂ is

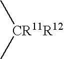

### or O or

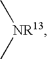

then 95% TFA/H₂O. (f) Where Z is

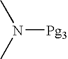

### and P₂ is

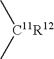

### or O or

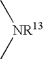

and ‘Pg₃’=Boc then 35% TFA in DCM. (g) range of chemistries to couple R¹COOH or R²SO₂Cl or R¹OCOCl or R¹N═C═O or R³COCl, then 95% TFA/H₂O.

The protected building blocks (9a,b-14a,b) may be utilised in a solid phase synthesis of compounds of general formula (I) following steps (b) to (e). Preferred protecting group combinations include ‘Pg₁’=Fmoc/‘Pg₂’-Boc, or ‘Pg₁’=Fmoc/‘Pg₂’=R¹³, or ‘Pg₁’=Pmoc/‘Pg₂’=R¹³/‘Pg₃’=Boc, or ‘Pg₁’=Fmoc/‘Pg₃’=Boc. General utilisation of ketone building blocks (9a-14a) in a solid phase syntheses is exemplified in Scheme 1.

Within step (b), reversible solid phase linkage of a ketone, has previously been described by a variety of methods (e.g. see (i) James, I. W., 1999, (ii) Lee, A., Huang, L., Ellman, J. A., J. Am. Chem. Soc, 121(43), 9907-9914, 1999, (iii) Murphy, A. M., et al, J. Am. Chem. Soc, 114, 3156-3157, 1992). A suitable method amenable to the reversible linkage of an alkyl ketone functionality such as (9a-14a) is through a combination of the previously described chemistries utilising the semicarbazide, 4-[[(hydrazinocarbonyl)amino]methyl]cyclohexane carboxylic acid. trifluoroacetate (Murphy, A. M., et al, J. Am. Chem. Soc, 11, 3156-3157, 1992), (see (i) WO-A-02057270, (ii) WO-A-04007501, (iii) Quibell, M. et. al., Bioorg. Med. Chem. 12, 5689-5710, 2004, (iv) Quibell, M. et. al., Bioorg. Med. Chem. 13, 609-625, 2005).

Alternatively, the analogous bicyclic alcohols of intermediates (9b-14b) may be utilised where the secondary alcohol may be attached to the solid phase through the acid labile dihydropyran linker that is well known in the literature (e.g. see (a) Thompson, L. A. and Ellman, J. A, Tet. Lea., 5, 9333, 1994. (b) Kick, E. K. and Ellman, J. A. J. Med. Chem., 38, 1427, 1995). Solid phase synthesis then proceeds as detailed in Scheme 1 and following acidolytic cleavage of the secondary alcohol into solution, final oxidation (e.g. Dess-Martin periodinane in DCM or solid supported oxidants e.g. see Ley, S. V. et al, J. Chem. Soc. Perkin Trans. 1., 3815-4195, 2000) provides compounds of general formula (I).

Within step (c), preferred solid phase chemistry for introduction of the (U)ₘ—(X)ₙ—(Y₁)ₒ. Y₂-group is through the use of chloroformates (Y₂ is O, (U)ₘ—(X)ₙ—(Y₁)ₒ—SC(O)Cl (18)), chlorothiolformates (Y₂ is S, (U)ₘn—(X)ₙ—(Y₁)ₒ—SC(O)Cl (19)), isocyanates (Y₂ is NH, (U)ₘ—(X)ₙ—(Y₁)ₒ—N═C═O (20)) or carbamoyl chlorides (Y₂ is NR¹⁴, (U)ₘ—(X)ₙ—(Y₁)ₒ—NHCOCl (21)) as a single step reaction. Where Z=‘Pg₃’, orthogonal removal with approximately 35% TFA in DCM then liberates the secondary amine functionality of the right-hand ring, which may be acylated with a range of R¹COOH carboxylic acids, R²SO₂Cl sulphonyl chlorides, R¹N═C═O isocyanates, R¹OCOCl chloroformates or R³COCl carbamoylchlorides to provide

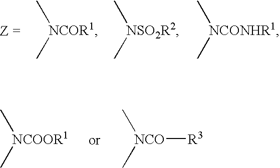

respectively. Finally, loaded constructs e.g. (17) may be treated with 95% TFA/H₂O to release compounds of general formula (I) from the solid phase.

A second strategy for the synthesis of compounds of general formula (I) comprises:—
(a) Preparation of an appropriately functionalised and protected bicyclic intermediate (9a,b,c-14a,b,c) building block in solution. Preferred protecting groups for solution phase chemistry are the 9-fluorenylmethoxycarbonyl (Fmoc), Nα-tert-butoxycarbonyl (Boc), Nα-benzyloxycarbonyl (Cbz) and Nα-allyloxycarbonyl group (Alloc).
(b) Standard organic chemistry methods for the conversion of building block obtained in step (a) towards compounds of general formula (I).

In the simplest example, the entire left hand portion of a compound of general formula (I) (i.e. the (U)ₘ—(X)ₙ—(Y₁)ₒ—Y₂— group) can be prepared in solution by traditional organic chemistry methods and coupled (e.g. via chloroformates (U)ₘ—(X)ₙ—(Y₁)ₒ—OC(O)Cl (18), chlorothiolformates (U)ₘ—(X)ₙ—(Y₁)ₒ—SC(O)Cl (19), isocyanates (U)ₘ—(X)ₙ—(Y₁)ₒN═C═O (20)) or carbamoyl chlorides (U)ₘ—(X)ₙ—(Y₁)ₒ—NHCOCl (21) to ketone, alcohol or ketal intermediates such as compounds (22), (23) and (24) where the ‘Z’ variant is already in place i.e. Z=O,

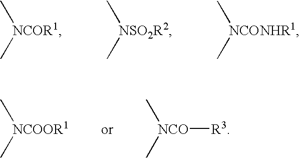

Then oxidation of the alcohol intermediate (e.g. Dess-Martin periodinane in DCM) or acidolytic cleavage of the ketal intermediate provides compounds of general formula (I). The alcohol oxidation route is particularly useful when the compound of general formula (I) contains a substituent that is labile to trifluoroacetic acid, this being the final reagent used in each of the solid phase Schemes 1 and 2.

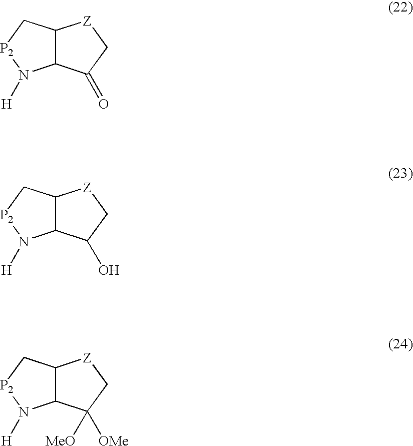

Variations upon this synthetic strategy involve coupling of the entire left hand portion of a compound of general formula (I) (i.e. the (U)ₘ—(X)ₙ—(Y₁)ₒ—Y₂— group) to intermediates (22), (23) or (24) where Z=‘Pg3’, followed by conversion of Z=‘Pg₃’ into Z=

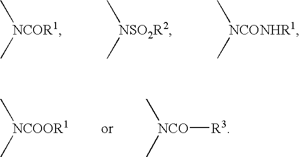

Examples of these different coupling tactics have been detailed previously (see (i) Quibell, M. et. al., Bioorg. Med. Chem. 13, 609-625, 2005. (ii) Wang, Y. et. al., Bioorg. Med. Chem. Lett. 15, 1327-1331, 2005) and the optimum synthetic route is dependant upon the specific substituent combinations of the target compound of general formula (I).

The invention extends to novel intermediates as described above, and to processes for preparing compounds of general formula (I) from each of their immediate precursors. In turn, processes for preparing intermediates from their immediate precursors also form part of the invention.

The present invention is further described by way of example.

### EXAMPLES

### Experimental Procedures

### Solution Phase Chemistry—General Methods

All solvents were purchased from ROMIL Ltd (Waterbeach, Cambridge, UK) at SpS or Hi-Dry grade unless otherwise stated. General peptide synthesis reagents were obtained from Chem-Impex Intl. Inc. (Wood Dale Ill. 60191. USA). Thin layer chromatography (TLC) was performed on pre-coated plates (Merck aluminium sheets silica 60 F254, part no. 5554). Visualisation of compounds was achieved under ultraviolet light (254 nm) or by using an appropriate staining reagent. Flash column purification was performed on silica gel 60 (Merck 9385) or Isolute Flash silica cartridge. All analytical HPLC were obtained on Phenomenex Jupiter C₄, 5μ, 300 A, 250×4.6 mm, using mixtures of solvent A=0.1% aq trifluoroacetic acid (TFA) and solvent B=90% acetonitrile /10% solvent A on automated Agilent systems with 215 and/or 254 mm UV detection. Unless otherwise stated a gradient of 10-90% B in A over 25 minutes at 1.5 mL 1 min was performed for full analytical HPLC analysis. HPLC-MS analysis was performed on an Agilent 1100 series LC/MSD, using automated Agilent HPLC systems, with a gradient of 10-90% B in A over 10 minutes on Phenomenex Columbus C₈, 5μ, 300 A, 50×2.0 mm at 0.4 mL/min. Nuclear magnetic resonance (NMR) were obtained on a Bruker DPX₄₀₀ (400 MHz 1H frequency; QXI probe) or Bruker DPX500 (500 MHz 1H frequency) in the solvents and temperature indicated (298K unless otherwise stated). Chemical shifts are expressed in parts per million (3) and are referenced to residual signals of the solvent. Coupling constants (J) are expressed in Hz. High resolution mass spectrometry was performed on a Micromass QTOF 1.

### Solid Phase Chemistry—General Methods

Example inhibitors were prepared through a combination of solution and solid phase Fmoc-based chemistries (see (i) Grabowska, U. et al, J. Comb. Chem. 2(5). 475-490, 2000 for a detailed description of solid phase multipin methodologies and (ii) WO-A-02057270 and (iii) WO-A-04007501 for general applications towards bicyclic ketones). An appropriately protected and functionalised building block was prepared in solution (e.g. intermediates (9a-14a)), then reversibly attached to the solid phase through an appropriate linker followed by rounds of coupling/deprotection/chemical modification (e.g. see Scheme 1). Example inhibitors were then released (cleaved) from the solid phase, analysed, purified and assayed for inhibition against a range of proteinases.

Generally, multipins (polyamide 1.3→10 μpmole loadings, see www.mimotopes.com) were used for the solid phase synthesis, although any suitable solid phase surface could be chosen. In general, the 1.3 μmole gears were used to provide small scale crude examples for preliminary screening, whilst the 10 μmole crowns were used for scale-up synthesis and purification of preferred examples. Standard coupling and Fmoc deprotection methods were employed (see Grabowska, U. et al, J. Comb. Chem. 2(5). 475-490, 2000 for a thorough description of solid phase multipin methodologies).

### Preparation of Initial Assembly

Building block-linker constructs (e.g. (15), typically 10 mg to 1000 mg) were carboxyl activated with 2-(1H-benzotriazole-1-yl)-1,1,3,3-tetramethyluronium hexafluoro phosphate (HBTU, 1 mole equivalent), 1-hydroxybenzotriazole.hydrate (HOBT, 1 mole equivalent) and N-methylmorpholine , 2 mole equivalents) in dimethylformamide (DMF, typically 1 to 10 mL) for 5 minutes. Amino functionalised DA/MDA crowns or lanterns or HEMA gears (101 mole per crown/8 μmole per lantern/1.3 μmole per gear, 0.33 mole equivalent of total surface amino functionalisation compared to activated construct) were added, followed by additional DME to cover the solid phase surface. The loading reaction was left overnight. Following overnight loading, crowns/lanterns/gears were taken through standard cycles washing, Fmoc deprotection and loading quantification (see Grabowska, U. et al, J. Comb. Chem. 2(5). 475-490, 2000) to provide loaded building block-linker constructs (e.g. free secondary amine analogue of intermediate (16)).

### Coupling Cycles

(i) The coupling of chloroformates R¹OCOCl or (U)ₘ—(X)ₙ—(Y₁)ₒ—OC(O)Cl (10 or 20 mole equivalent) was performed with N-methylmorpholine , 5 or 10 mole equivalents) in anhydrous THF and the reaction left overnight. Following overnight coupling, crowns/lanterns/gears were then washed (3 min. each) with 4×DMF, 4× acetonitrile and dried in vacuo.

(ii) The coupling of chlorothiolformates (U)ₘ—(X)ₙ(Y₁)ₒ—SC(O)Cl (10 or 20 mole equivalent) was performed with N-methylmorpholine (NMM, 5 or 10 mole equivalents) in anhydrous THF and the reaction left overnight. Following overnight coupling, crowns/lanterns/gears were then washed (3 min. each) with 4×DMF, 4× acetonitrile and dried in vacuo.

(iii) The coupling of isocyanates R¹N═C═O or (U)ₘ—(X)ₙ—(Y₁)ₒ—N═C═O (10 or 20 mole equivalent) was performed in DMF and the reaction left overnight. Following overnight coupling, crowns/lanterns/gears were then washed (3 min. each) with 4×DMF, 4× acetonitrile and dried in vacuo.

(iv) The coupling of carbamoylchlorides R³COCl or (U)ₘ—(X)ₙ(Y₁)ₒ—NR¹⁴C(O)Cl (10 or 20 mole equivalent) was performed with N-methylmorpholine (NMM, 5 or 10 mole equivalents) in DMF and the reaction left overnight. Following overnight coupling, crowns/lanterns/gears were then washed (3 min each) with 4×DMF, 4× acetonitrile and dried in vacuo.

(v) The coupling of carboxylic acids R¹COOH (10 or 20 mole equivalent) was performed via carboxyl activated with 2-(1H-benzotriazole-1-yl)-1,1,3,3-tetramethyluronium hexafluoro phosphate (HBTU, 10 or 20 mole equivalent), 1-hydroxybenzotriazole.hydrate (HOBT, 10 or 20 mole equivalent) and N-methylmorpholine (NMM, 20 or 40 mole equivalents) in DMF, with pre-activation for 5 minutes.

(vi) The coupling of sulphonyl chlorides R²SO₂Cl (50 mole equivalent) was performed with dimethylaminopyridine (DMAP, 50 mole equivalent) in anhydrous THF and the reaction left overnight. Following overnight coupling, crowns/lanterns/gears were then washed (3 min. each) with 4×DMF, 4× acetonitrile and dried in vacuo.

Appropriate activated species were dispensed to the appropriate wells of a polypropylene 96-well plate (Beckman, 1 mL wells, 500 μL solution per well for crowns and lanterns or 250 μL solution per well for gears) in a pattern required for synthesis and the coupling reaction left overnight. Following overnight coupling, crowns /lanterns/gears were then washed (3 min. each) with 4×DMF, 4× acetonitrile and dried in vacuo.

### Graduated Acidolytic Cleavage of Boc Protecting Groups

Solid phase assemblies containing Z=N-Pg₃, where ‘Pg₃’=Boc (e.g. analogues of intermediates (16) and (17), Scheme 2) were treated with 35% trifluoroacetic acid (TFA) in DCM for 30 min. The Boc-deprotected crowns/lanterns/gears were then washed (3 min. each) with 1×DCM, 1×DMF, 1×2% v/v N-methylmorpholine in DMF, 4×DMF, 4× acetonitrile and dried in vacuo.

### Full Acidolytic Cleavage Cycle

A mixture of 95% TFA/5% water was pre-dispensed into two polystyrene 96-well plates (Beckman, 1 mL wells, 600 μL solution per well for crowns and lanterns or 300 μL solution per well for gears) in a pattern corresponding to that of the synthesis. The completed multipin assembly was added to the first plate (mother plate), the block covered in tin foil and cleaved for 2 hours. The cleaved multipin assembly was then removed from the first plate and added to the second plate (washing plate) for 15 minutes. The spent multipin assembly was then discarded and the mother/washing plates evaporated on an HT4 GeneVac plate evaporator.

### Analysis and Purification of Cleaved Examples

(a) Ex 1.3 μmole gears. 100 μL dimethylsulphoxide (DMSO) was added to each post cleaved and dried washing plate well, thoroughly mixed, transferred to the corresponding post cleaved and dried mother plate well and again thoroughly mixed. 10 μL of this DMSO solution was diluted to 100 μL with a 90% acetonitrile /10% 0.1% aq TFA mixture. 20 μL aliquots were analysed by HPLC-MS and full analytical HPLC. In each case the crude example molecules gave the expected [M+H]⁺ ion and an HPLC peak at >80% (by 215 n UV analysis). This provided an approximately 10 mM DMSO stock solution of good quality crude examples for preliminary proteinase inhibitory screening.
(b) Ex 10 μmole crowns or 8 μmole lanterns. 500 μL of a 90% acetonitrile/10% 0.1% aq TFA mixture was added to each washing plate well, thoroughly mixed, transferred to the corresponding mother plate well and again thoroughly mixed. 5 μL of this solution was diluted to 100 μL with a 90% acetonitrile/10% 0.1% aq TFA mixture. 20 μL aliquots were analysed by HPLC-MS and full analytical HPLC. In each case the crude example molecules gave the expected [M+H]⁺ ion and an HPLC peak at >80% (by 215 mm UV analysis). The polystyrene blocks containing crude examples were then lyophilised.
(c) Individual examples (ex (b)) were re-dissolved in a 1:1 mixture of 0.1% aq TFA /acetonitrile (1 mL) and purified by semi-preparative HPLC (Phenomenex Jupiter C₄, 5μ, 300 A, 250×10 mm, a 25-90% B in A gradient over 25 min., 4.0 mL/min, 215 nm UV detection). Fractions were lyophilised into pre-tarred glass sample vials to provide purified examples (typically 2 to 4 mg, 40 to 80% yield).
(d) Purified examples were dissolved in an appropriate volume of DMSO to provide a 10 mM stock solution, for accurate proteinase inhibitory screening.

EXAMPLES 1-51 were prepared using the general solid phase descriptions above and are inhibitors of cathepsin K with Ki <50 μM;

### Example 1

### (3aR,6aS)-4-Benzoyl-6-oxo-hexahydro-pyrrolo[3,2-b]pyrrole-1-carboxylic acid 1-isopropyl-2-methyl-propyl ester [9]

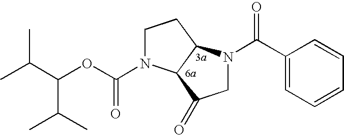

Through the use of 2,4-dimethyl-pentan-3-chloroformate.

HPLC-MS Rt=3.35 mins (>85%), 373.2 [M+H]⁺.

### Example 2

### (3aR,6aS)-4-(2S-Acetylamino-4-methyl-pentanoyl)-6-oxo-hexahydro-pyrrolo[3,2-b]pyrrole-1-carboxylic acid 1-isopropyl-2-methyl-propyl ester [10]

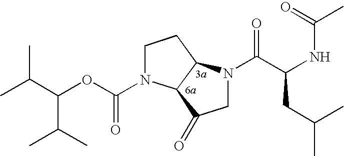

Through the use of 2,4-dimethyl-pentan-3-chloroformate.

HPLC-MS Rt=7.00 mins (>90%), 424.3 [M+H]⁺.

### Example 3

### (3aR,6aS)-4-(2S-Acetylamino-4-methyl-pentanoyl)-6-oxo-hexahydro-pyrrolo[3,2-b]pyrrole-1-carboxylic acid isobutyl ester [11]

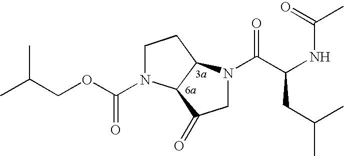

Through the use of isobutylchloroformate.

HPLC-MS Rt=5.84 mins (>90%), 382.3 [M+H]⁺.

### Example 4

### (3aR,6aS)-4-(2S-Acetylamino-4-methyl-pentanoyl)-6-oxo-hexahydro-pyrrolo[3,2-b]pyrrole-1-carboxylic acid isopropyl ester [12]

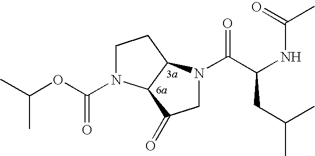

Through the use of isopropylchloroformate.

HPLC-MS Rt=5.01 mins (>90%), 368.2 [M+H]⁺.

### Example 5

### (3aR,6aS)-4-(2S-Acetylamino-4-methyl-pentanoyl)-6-oxo-hexahydro-pyrrolo[3,2-b]pyrrole-1-carboxylic acid 2,2-dimethyl-propyl ester [13]

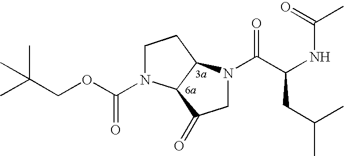

Through the use of 2,2-dimethylpropyl-3-chloroformate.

HPLC-MS Rt=6.47 mins (>90%), 396.3 [M+H]⁺.

### Example 6

### (3aR,6aS)-4-(2S-Acetylamino-4-methyl-pentanoyl)-6-oxo-hexahydro-pyrrolo[3,2-b]pyrrole-1-carboxylic acid diethylamide [14]

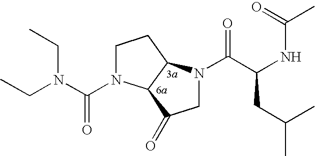

Through the use of diethylcarbamoyl chloride.

HPLC-MS Rt=4.60 mins (>85%), 381.3 [M+H]⁺.

### Example 7

### (3aR,6aS)-4-(2S-Acetylamino-4-methyl-pentanoyl)-6-oxo-hexahydro-pyrrolo[3,2-b]pyrrole-1-carboxylic acid sec-butylamide [15]

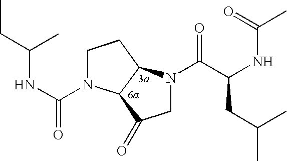

Through the use of sec-butylisocyanate.

HPLC-MS Rt=4.69 mins (>85%), 381.3 [H+H]⁺.

### Example 8

### (3aR,6aS)—N-{3-Methyl-1-[3-oxo-4-pyrrolidine-1-carbonyl)-hexahydro-pyrrolo[3,2-b]pyrrole-1-carbonyl]-butyl}-acetamide [16]

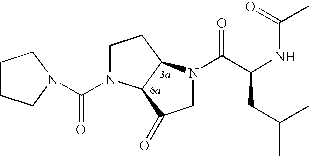

Through the use of 1-pyrrolidinecarbonyl chloride.

HPLC-MS Rt=4.13 mins (>85%), 379.3 [M+H]⁺.

### Example 9

### (3aR,6aS)-4-Benzoyl-6-oxo-hexahydro-pyrrolo[3,2-b]pyrrole-1-carboxylic acid 4,4-dimethyl-2-oxo-tetrahydro-furan-3R-yl ester [17]

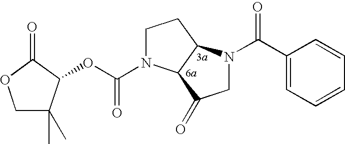

Through the use of (R)-pantolactone chloroformate.

HPLC-MS Rt=3.54 mins (>90%), 387.1 [M+H]⁺.

### Example 10

### (3aR,6aS)-4-Benzoyl-6-oxo-hexahydro-pyrrolo[3,2-b]pyrrole-1-carboxylic acid 4,4-dimethyl-2-oxo-tetrahydro-furan-3S-yl ester [18]

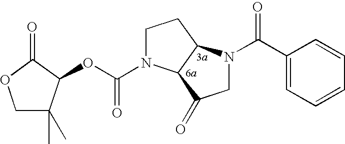

Through the use of (S)-pantolactone chloroformate.

HPLC-MS Rt=3.68 mins (>90%), 387.2 [M+H]⁺.

### Example 11

### (3aS,6aR)-3-Oxo-hexahydro-furo[3,2-b]pyrrole-4-carboxylic acid 2,2-dimethyl-propyl ester [19]

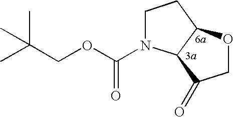

Through the use of 2,2-dimethylpropyl-3-chloroformate.

HPLC-MS Rt=4.85 mins (>90%), 242.1 [M+H]⁺.

### Preparation of Alcohols

### 3-Methyl-1-phenylbutan-1-ol (ALCOHOL 1)

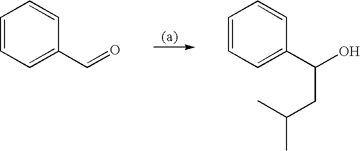

(a) Isobutylmagnesium Bromide, THF.

Isobutylmagnesium bromide (2M in diethyl ether, 49.4 mL, 98.9 mmol) was added dropwise to a solution of benzaldehyde (8.75 g, 82.4 mmol) in tetrahydrofuran (50 mL) at 0° C. with stirring under an atmosphere of nitrogen over 35 minutes then allowed to warm to ambient temperature over 18 hours. Saturated ammonium chloride solution (90 mL) was added then the mixture extracted with ethyl acetate (100 mL then 2×50 mL), then reduced in vacuo. The yellow-red residue was purified by flash chromatography over silica, eluting with ethyl acetate:hexane mixtures 20:80 to give 3-methyl-1-phenylbutan-1-ol as a pale yellow oil (6.77 g, 50%). TLC (R_{f}=0.40, EtOAc:hexane 3:7).

5-Methyl-1-(thiophen-2-yl)hexan-3-ol (ALCOHOL 2)

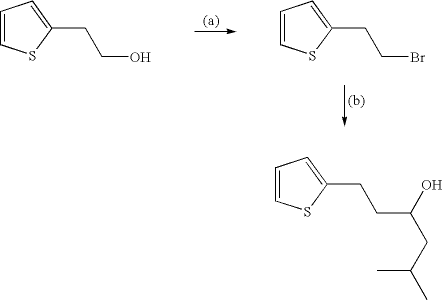

(a) PBr₃, CCl₄; (b) Mg, 3-methylbutanal, ThIF, I₂.

Phosphorus tribromide (2.0 mL, 21.1 mmol) was added to a stirred solution of 2-(thiophen-2-yl)ethanol (2.25 g, 17.6 mmol) in carbon tetrachloride (162 mL) then the mixture heated at 65° C. for 20 minutes. The mixture was allowed to cool to ambient temperature then ice added. The organic layer was separated then the aqueous layer extracted with dichloromethane (2×30 mL). The combined organic layers were washed with brine, then dried (NaSO₄), filtered and reduced in vacuo. The residue was purified by flash chromatography over silica, eluting with ethyl acetate:hexane mixtures 0:100 to 0.5:95.5 to give 2-(2-bromoethyl)thiophene as a brown oil (650 mg, 19%).

One iodine crystal was added to a stirred suspension of magnesium turnings (45 mg, 1.85 mmol) in tetrahydrofuran (10 mL) then the mixture was heated at reflux. A solution of 2-(2-bromoethyl)thiophene (300 mg, 1.57 mmol) in tetrahydrofuran (3 mL) was added dropwise over 10 minutes then the mixture heated at reflux for 4 hours. The mixture was cooled to 0° C. then 3-methylbutanal (0.16 mL, 1.57 mmol) was added over 30 minutes then stirred at ambient temperature for 18 hours. Aqueous hydrochloric acid was added until pH<7 then the mixture was extracted with ethyl acetate (40 mL, 20 mL then 10 mL), then dried (NaSO₄), filtered and reduced in vacuo. The residue was purified by flash chromatography over silica, eluting with ethyl acetate:hexane mixtures 2:98 to 4:96 to give 5-methyl-1-(thiophen-2-yl)hexan-3-ol as a colourless oil (93 mg, 30%). HPLC-MS 199.1 [M+H]⁺; δ_{H} (300 M , CDCl₃) 0.90 (6H, m, CH(CH₃)₂), 1.10-1.90 (6H, m, CH₂CH(OH)CH₂), 2.75-3.00 (2H, m, CH₂CH₂C(OH)), 3.65 (1H, brs, OR), 6.75 (1H, m, SCHCHCH), 6.85 (1H, m, SCHCHCH), 7.00 (1H, d, J=4.5 Hz, SCHCHCH).

### 1-Benzylcyclobutanol (ALCOHOL 12)

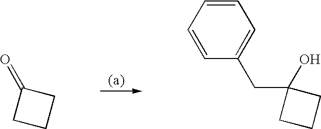

(a) 1M BnMgCl in Et₂O, THF.

Following literature preparation detailed in WO03062192 (pg 36).

1-Phenethylcyclobutanol (ALCOHOL 19)

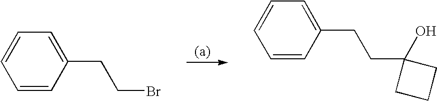

(a) Mg, cyclobutanone, THF, 12.

One iodine crystal was added to a stirred suspension of magnesium turings (45 mg, 1.85 mmol) in tetrahydrofuran (7 mL) then the mixture heated at reflux. (2-Bromoethyl)benzene (500 mg, 2.70 mmol) was added dropwise over 45 minutes then the mixture was heated at reflux for 4 hours. The mixture was cooled to 0° C. then cyclobutanone (189 mg, 2.70 mmol) was added over 30 minutes and the mixture stirred at ambient temperature for 18 hours. Aqueous hydrochloric acid was added until pH<7 then the mixture was extracted with ethyl acetate (3×20 mL), then dried (NaSO₄), filtered and reduced in vacuo. The residue was purified by flash chromatography over silica, eluting with ethyl acetate:hexane mixtures 0:100 to 1.5:98.5 to give 1-phenethylcyclobutanol as a white solid (200 mg, 62%). TLC (R_{f}=0.30, EtOAc hexane 1:4), analytical HPLC single main peak, R_{f}=12.78 min., HPLC-MS 159.2 [M-OH]⁺; δ_{H} (300 MHz D₆-DMSO) 1.40-2.00 (8H, m, CH₂C(OB) and CH₂CH₂CH₂), 2.60-2.65 (2H, m, SCCH₂), 5.00 (1H, s, OR), 7.10-7.30 (5H, m, aromatic CH).

### 5,5-Dimethylhexan-3-ol (ALCOHOL 28)

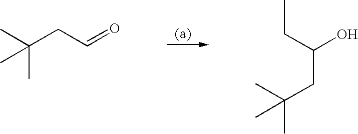

(a) EtMgBr, Et₂O.

Ethylmagnesium bromide (3M in diethyl ether, 13.0 mL, 38.9 mmol) was added dropwise to a stirred solution of 3,3-dimethylbutanal (3.0 g, 30.0 mmol) in diethyl ether (40 mL) at −10° C. under an atmosphere of nitrogen over 2 hours. The mixture was allowed to warm to ambient temperature over 18 hours then poured into saturated aqueous ammonium chloride solution (10 mL), then extracted with ethyl acetate (30 mL then 2×20 mL) then dried (NaSO₄), filtered and reduced in vacuo. The residue was purified by flash chromatography over silica, eluting with ethyl acetate:hexane mixtures 1:9 to give 5,5-dimethylhexan-3-ol as a colourless oil (500 mg, 13%). TLC (R_{f}=0.35, EtOAc:hexane 1:3); OH (300 M , CDCl₃) 0.85-1.40 (7H, m, CH₃CH₂CHCH₂), 0.90 (9H, s, C(CH₃)₃), 1.60 (1H, s, O₁H), 3.55-3.60 (1H, m, CHOH); δ_{C} (75 MHz, CD₃OH) 10.31 (CH₃CH₂), 30.20 (C(CH₃)₃), 30.47 (C(CH₃)₃), 32.60 (CH₃CH₂), 51.08 ((CH₃)₃CCH₂), 71.15 (CHOH).

### 4-Ethylbiphenyl-3-ol (ALCOHOL 29)

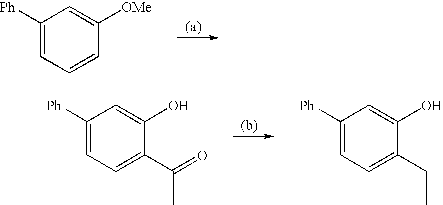

(a) AcCl, AlCl₃, DCM; (b) Zn/Hg, conc. HCl.

A solution of 3-methoxybiphenyl (2.42 g, 13.2 mmol) in dichloromethane (8 mL) was added dropwise to a stirred mixture of aluminium chloride (2.1 g, 15.7 mmol), acetyl chloride (0.94 mL, 13.2 mmol) and dichloromethane (8 mL) then the mixture heated at reflux for 6 hours. The mixture was poured onto ice (50 g) then hydrochloric acid (10%, 10 mL) added. The product was extracted into dichloromethane then solvents removed in vacuo. The residue was purified by fractional distillation at 0.1 mbar, 120-140° C. then recrystallized from hexane to obtain 1-(3-hydroxybiphenyl-4-yl)ethanone as a white needles (1.5 g, 50%). M.p. 90-92° C.

Concentrated hydrochloric acid (0.1 mL) was added to a mixture of zinc amalgam (freshly prepared from 1.2 g zinc and 1.5 g of mercuric chloride) and 1-(3-hydroxybiphenyl-4-yl)ethanone (420 mg, 1.98 mmol) in aqueous hydrochloric acid. The mixture was heated at reflux for 4 hours adding concentrated hydrochloric acid (3 drops) after each hour. The supernatant liquid was collected by decantation then extracted with ethyl acetate. The solvents were removed in vacuo then the residue recrystallised using petroleum ether (b.p. 40-60° C.) to give 4-ethylbiphenyl-3-ol as a white solid (210 mg, 54%). M.p. 62-65° C.

### (1-(Phenoxymethyl)cyclobutyl)methanol (ALCOHOL 30)

Synthesis of (1(benzyloxymethyl)cyclobutyl)methanol was carried out as detailed in WO03062192 (pg 95).

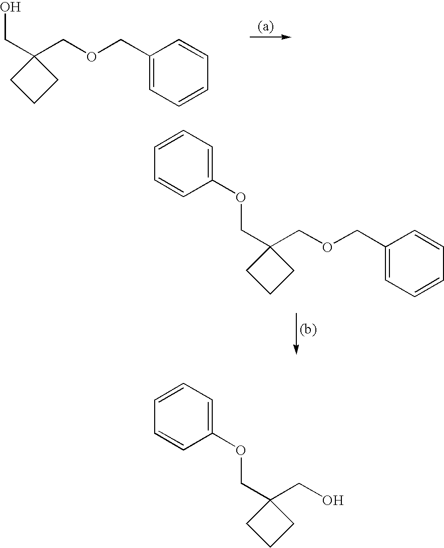

(a) Phenol, DIAD, PPh₃, THF; (b) 10% Pd/C, AcOH, MeOH, H₂.

Phenol (1.9 mg, 20.0 mmol) then triphenylphosphine (5.2 g, 20.0 mmol) were consecutively added to a solution of (1-(benzyloxymethyl)cyclobutyl)methanol (4.0 g, 19.4 mmol) in tetrahydrofuran (40 mL) under an atmosphere of nitrogen. The mixture was sonicated for 3 minutes whilst adding DIAD (4.0 g, 20.0 mmol). The mixture was sonicated for an additional 43 minutes then reduced in vacuo. The residue was purified by flash chromatography over silica, eluting with ethyl acetate:hexane mixtures 5:95 to give ((1-(benzyloxymethyl)cyclobutyl)methoxy)benzene as a yellow oil (3.2 g, 58%).

Hydrogen gas was bubbled through a mixture of 10% palladium on carbon (1.5 g) and ((1-(benzyloxymethyl)cyclobutyl)methoxy)benzene (3.0 g, 10.6 mmol) in methanol (50 mL) containing acetic acid (3 mL, 20.2 mmol) for 20 hours. The mixture was filtered then washed with methanol. The filtrate was reduced in vacuo to give (1-(phenoxymethyl)cyclobutyl)methanol as a colourless oil (1.3 g, 64%). TLC (R_{f}=0.30, EtOAc:hexane 1:3), HPLC-MS 175.1 [M-OH]⁺, 215.1 [M+Na]⁺; 8H (300 z, CDCl₃) 1.79-1.93 (6H, m, (CH₂)₃), 2.07 (1H, s, OH), 3.70 (2H, s, CH₂OH), 3.92 (2H, m, CH₂OPh), 6.80-6.89 (3H, m, aromatic CH), 7.14-7.23 (2H, m, aromatic CH).

### 1-Isopropylcyclopropanol (ALCOHOL 35)

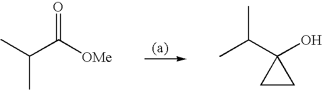

(a) 3M EtMgBr in Et₂O, titanium isopropoxide, Et₂O.

Following literature preparation detailed in Kulinkovich, 0. G. et al, Zhurnal Organicheskoi Khimii, 27(2), 294-8, 1991.

### 1-(Thiophen-3-yl)butan-2-ol (ALCOHOL 40)

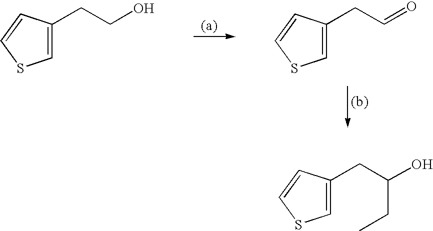

(a) Dess-Martin periodinane, DCM; (b) 3M EtMgBr in Et₂O, THF.

Dess-Martin periodinane (33.1 g, 78.1 mmol) was added to a solution of 2-(thiophen-3-yl)ethanol (10.0 g, 78.1 mmol) in dichloromethane (150 mL) under an atmosphere of nitrogen at 15° C. The mixture was stirred for 18 hours at ambient temperature then saturated aqueous sodium hydrogen carbonate solution was added until pH>7. The mixture was extracted with dichloromethane (200 mL then 100 mL), then dried (Na₂SO₄), filtered and reduced in vacuo to give 2-(thiophen-3-yl)acetaldehyde (8.5 g) which was used without further purification.

Ethylmagnesium bromide (3M in diethyl ether, 19.0 mL, 57.1 mmol) was added dropwise over 2 hours to a solution of 2-(thiophen-3-yl)acetaldehyde (6.0 g, assumed to be 47.6 mmol, prepared as above) in tetrahydrofuran (35 mL) at −10° C. with stirring under an atmosphere of nitrogen. The mixture was allowed to warm to ambient temperature over 18 hours then poured into saturated aqueous ammonium chloride solution, then extracted with ethyl acetate, then dried (NaSO₄), filtered and reduced in vacuo. The residue was purified by flash chromatography over silica, eluting with hexane followed by fractional distillation at 0.5 mm, 63-65° C. to give 1-(thiophen-3-yl)butan-2-ol as a colourless oil (1.6 g, 22%). TLC (R_{f}=0.27, EtOAc:hexane 1:4), HPLC-MS 139.1 [M-OH]⁺.

### 1-(Thiophen-2-yl)butan-2-ol (ALCOHOL 42)

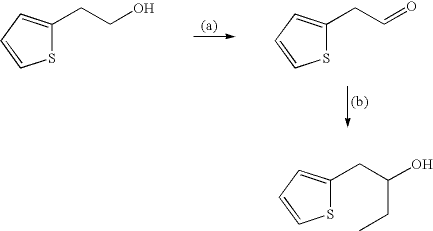

(a) Dess-Martin periodinane, DCM; (b) 3M EtMgBr in Et₂O, THF.

Dess-Martin periodinane (33.1 g, 78.1 mmol) was added to a solution of 2-(thiophen-2-yl)ethanol (10.0 g, 78.1 mmol) in dichloromethane (150 mL) under an atmosphere of nitrogen at 15° C. The mixture was stirred for 18 hours at ambient temperature then saturated aqueous sodium hydrogen carbonate solution was added until pH>7. The mixture was extracted with dichloromethane (2×100 mL), then dried (Na₂SO₄), filtered and reduced in vacuo to give 2-(thiophen-2-yl)acetaldehyde (4 g) which was used without further purification.

Ethylmagnesium bromide (3M in diethyl ether, 19.0 mL, 57.1 mmol) was added dropwise over 2 hours to a solution of 2-(thiophen-2-yl)acetaldehyde (6.0 g, assumed to be 47.6 mmol, prepared as above) in tetrahydrofuran (35 mL) at −10° C. with stirring under an atmosphere of nitrogen. The mixture was allowed to warm to ambient temperature over 18 hours then poured into saturated aqueous ammonium chloride solution (50 mL), then extracted with ethyl acetate (100 mL, 40 mL then 30 mL), then dried (NaSO₄), filtered and reduced in vacuo. The residue was purified by flash chromatography over silica, eluting with hexane followed by fractional distillation at 0.5 mm, 62° C. to give 1-(thiophen-2-yl)butan-2-ol as a colourless oil (500 mg, 7%). TLC (R_{f}=0.30, EtOAc:hexane 1:3), HPLC-MS 139.1 [M-OH]⁺, 157.1 (M+H⁺; δ_{H} (300 z CDCl₃) 0.95 (3H, t, J=6 Hz, CH₃), 1.40-1.60 (2H, m, CH₃CH₂), 1.85 (1H, s, OH), 2.80 (1H, dd, J=13 and 6 Hz, SCCH₂), 2.95 (1H, dd, J=13 and 3 Hz, SCCH₂), 3.60-3.75 (1H, m, CHOH), 6.80 (1H, brs, SCHCHCH), 6.90 (1H, m, SCHCHCH), 7.10 (1H, m, SCHCHCH).

### Example 12

### (3aR,6aS)-1-Benzylcyclobutyl 4-benzoyl-6-oxohexahydropyrrolo[3,2-b]pyrrole-1(2H)-carboxylate

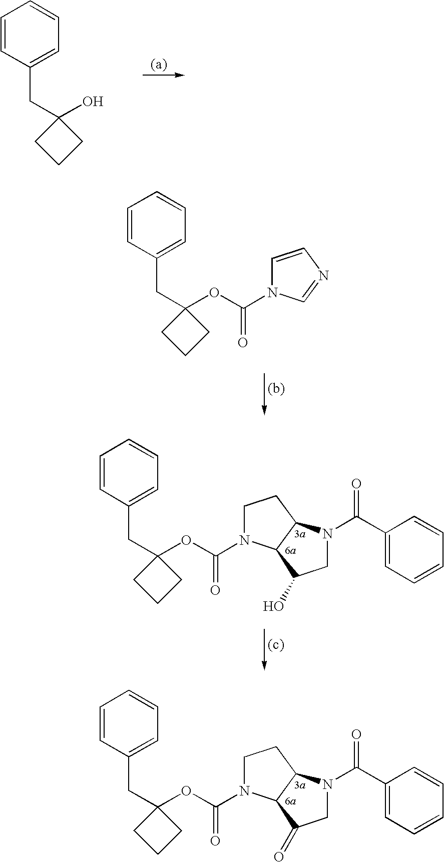

(a) CDI, DCM, 40° C.; (b) ((3S,3aS,6aR)-3-hydroxyhexahydropyrrolo[3,2-b]pyrrol-1(2H)-yl)(phenyl)methanone , DCM, 40-45° C., (c) Dess-Martin periodinane, DCM.

### Step 1

1,1′-Carbonyldiimidazole (114 mg, 0.705 mmol) in dichloromethane (1 mL) was added to 1-benzylcyclobutanol (ALCOHOL 12) (76 mg, 0.470 mmol). The mixture was heated at 40° C. for 17 hours then diluted with dichloromethane (10 mL). The solution was washed with water (7 mL) then dried (Na₂SO₄), filtered and reduced in vacuo to give 1-benzylcyclobutyl 1H-imidazole-1-carboxylate which was used without further purification.

### Step 2

A solution of 1-benzylcyclobutyl 1H-imidazole-1-carboxylate (assumed to be 0.188 mmol, prepared as above) in dichloromethane (1 mL) was added to ((3S,3aS,6aR)-3-hydroxyhexahydropyrrolo[3,2-b]pyrrol-1(2H)-yl)(phenyl)methanone (compound 31, Quibell, M. et al, Bioorg. Med. Chem. 13, 609-625, 2005), (50 mg, 0.215 mmol). The mixture was heated at 40° C. with stirring for 3 days, then at 45° C. for 7 days, then concentrated in vacuo. The residue was purified by short path flash chromatography over silica, eluting with ethyl acetate:heptane mixtures 0:100 to 100:0 to give (3aR,6S,6aS)-1-benzylcyclobutyl 4-benzoyl-6-hydroxyhexahydropyrrolo[3,2-b]pyrrole-1(2H)-carboxylate as a colourless oil (71 mg). TLC (R_{f}=0.24, EtOAc:heptane 3:2), analytical HPLC single main peak, Rₜ=15.25 min., HPLC-MS 421.2 [M+H]⁺, 863.3 [2M+Na]⁺.

### Step 3

(3aR,6S,6aS)-1-Benzylcyclobutyl 4-benzoyl-6-hydroxyhexahydropyrrolo[3,2-b]pyrrole-1(2H)-carboxylate (71 mg, assumed to be 0.17 mmol) in dichloromethane (1 mL) was added to Dess-Martin periodinane (143 mg, 0.34 mmol) under an atmosphere of argon. The mixture was stirred for 18 hours then diluted with dichloromethane (10 mL). The mixture was then washed with a mixture of 0.5M sodium thiosulphate:saturated aqueous sodium hydrogen carbonate (1:1, 8 mL), then saturated aqueous sodium hydrogen carbonate (8 mL), then brine (8 mL), then water (8 mL), then dried (Na₂SO₄), filtered and reduced in vacuo. Preparative thin layer chromatography eluting with ethylacetate:heptane 3:2 gave (3aR,6aS)-1-benzylcyclobutyl 4-benzoyl-6-oxohexahydropyrrolo[3,2-b]pyrrole-1(2H)-carboxylate as a colourless oil (24 mg, 34%). TLC (R_{f}=0.46, EtOAc heptane 3:2), analytical HPLC broad main peak Rₜ=15.18-16.99 min., HPLC-MS 275.1 [M-PhCH₂⁰Bu+H]⁺, 419.2 [M+H]⁺, 437.2 [M+H₂O+H]⁺, 441.2 [M+Na]⁺, 459.2 [M+H₂O+Na]⁺, 859.3 [2M+Na]⁺, 895.3 [2(M+H₂O)+Na]⁺.

### Example 13

### (3aR,6aS)-1-Phenethylcyclobutyl 4-benzoyl-6-oxohexahydropyrrolo[3,2-b]pyrrole-1(2H)-carboxylate

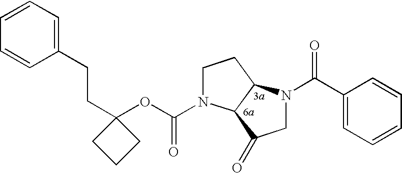

As detailed for EXAMPLE 12 but through use of 1-phenethylcyclobutanol (ALCOHOL 19).

TLC (R_{f}=0.52, EtOAc:heptane 3:2), analytical HPLC broad main peak, Rₜ=16.73-18.02 min., HPLC-MS 275.1 [M-Ph(CH₂)₂^{c}Bu+H]⁺, 451.2 [M+H₂O+H]⁺, 455.2 [M+Na]⁺, 887.3 [2M+Na]⁺, 923.3 [2(M+H₂O)+Na]₃⁺.

### Example 14

### (3aR,6aS)-1-(Thiophen-3-yl)butan-2-yl 4-benzoyl-6-oxohexahydropyrrolo[3,2-b]pyrrole-1(2H)-carboxylate

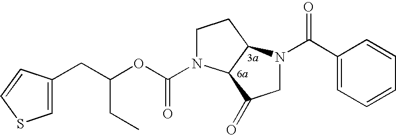

As detailed for EXAMPLE 12 but through use of 1-(thiophen-3-yl)butan-2-ol (ALCOHOL 40).

TLC (R_{f}=0.48, EtOAc:heptane 3:2), analytical HPLC broad main peak, Rₜ=14.58-16.60 min., HPLC-MS 413.2 [M+H]⁺, 431.2 [M+H₂O+H]⁺, 847.2 [2M+Na]⁺, 883.3 [2(M+H₂O)+Na]⁺.

### Example 15

### (3aR,6aS)-(1-(Phenoxymethyl)cyclobutylmethyl 4-benzoyl-6-oxohexahydropyrrolo[3,2-b]pyrrole-1(2M)-carboxylate

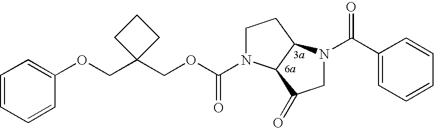

As detailed for EXAMPLE 12 but through use of (1-(phenoxymethyl)cyclobutyl)methanol (ALCOHOL 30) with Step 1 reaction time adjusted to 3 hours and Step 2 reaction time adjusted to 14 days at 40° C.

Analytical HPLC main peaks, Rₜ=18.73-20.25 min., HPLC-MS 449.2 [M+H]⁺, 471.2 [M+H]⁺ hydrate, 471.2 [M+Na]⁺, 919.3 [2M+Na]⁺.

### Example 16

### (3aR,6aS)-1-(Thiophen-2-yl)butan-2-yl 4-benzoyl-6-oxohexahydropyrrolo[3,2-b]pyrrole-1(2H)-carboxylate

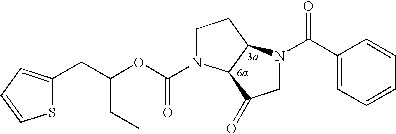

As detailed for EXAMPLE 12 but through use of 1-(thiophen-2-yl)butan-2-ol (ALCOHOL 42) with Step 1 reaction time adjusted to 15 hours and Step 2 reaction time adjusted to 12 days at 40° C.

Analytical HPLC main peaks, Rₜ=17.18-18.64 min., HPLC-MS 413.1 [M+H]⁺, 431.1 [M+H₂O+H]⁺, 847.2 [2M+Na]⁺, 883.3 [2(M+H₂O)+Na]⁺.

### Example 17

### (3aR,6aS)-1-Isopropylcyclopropyl benzoyl-6-oxohexahydropyrrolo[3,2-b]pyrrole-1(2H-carboxylate

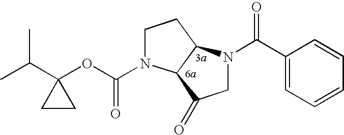

As detailed for EXAMPLE 12 but through use of 1-isopropylcyclopropanol (ALCOHOL 35) with Step 1 reaction time adjusted to 3 hours and Step 2 reaction time adjusted to 12 days at 40° C.

Analytical HPLC main peaks, Rₜ=14.96-16.25 min., HPLC-MS 357.2 [M+H]⁺, 375.2 [M+H₂O+H]⁺, 735.3 [2M+Na]⁺, 771.3 [2(M+H₂O)+Na]⁺.

### Example 18

### (3aR,6aS)-5-Methyl-1-(thiophen-2-yl)hexan-3-yl 4-benzoyl-6-oxohexahydropyrrolo[3,2-b]pyrrole-1(2H)-carboxylate

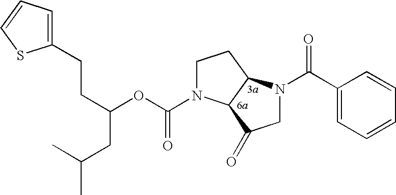

As detailed for EXAMPLE 12 but through use of 5-methyl-1-(thiophen-2-yl)hexan-3-ol (ALCOHOL 2) with Step 1 reaction time adjusted to 3 hours and Step 2 reaction time adjusted to 19 days at 40° C.

Analytical HPLC broad main peak, Rₜ=20.39-21.71 min., HPLC-MS 455.2 [M+H]⁺, 931.3 [M+Na]⁺, 967.4 [2(M+H₂O)+Na]⁺.

### Example 19

### (3aR,6aS)-5,5-Dimethylhexan-3-yl 4-benzoyl-6-oxohexahydropyrrolo[3,2-b]pyrrole-1 (2H)-carboxylate

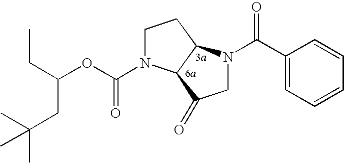

As detailed for EXAMPLE 12 but through use of 5,5-dimethylhexan-3-ol (ALCOHOL 28) with Step 1 reaction time adjusted to 2 hours and Step 2 reaction time adjusted to 17 days at 40° C.

Analytical HPLC main peaks, Rₜ=18.96-19.89 min., HPLC-MS 387.2 [M+H]⁺, 795.4 [2M+Na]⁺.

### Example 20

### (3aR,6aS-3-Methyl-1-phenylbutyl 4-benzoyl-6-oxohexahydropyrrolo[3,2-b]pyrrole-1(2H)-carboxylate

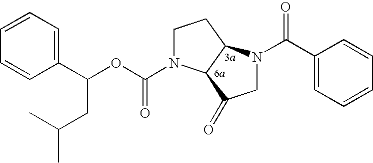

As detailed for EXAMPLE 12 but through use of 3-methyl-1-phenylbutan-1-ol (ALCOHOL 1) with Step 1 employing 2 mole equivalents of 1,1′-carbonyldiimidazole and reaction time adjusted to 2 days, and Step 2 reaction time adjusted to 6 days at 40° C.

Analytical HPLC main peaks, Rₜ=19.06-20.52 min., HPLC-MS 439.2 [M+H₂O+H]⁺, 863.4 [2M+Na]⁺, 899.4 [2(M+H₂O)+Na]⁺.

### Example 21

### (3aS,6aR)-1-Benzylcyclobutyl 3-oxotetrahydro-2H-furo[3,2-b]pyrrole-4(5H)-carboxylate

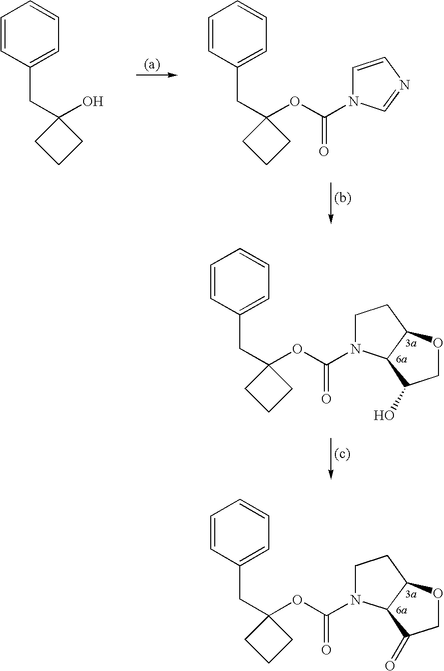

(a) CDI, DCM, 40° C.; (b) (3R,3aR,6aR)-hexahydro-2H-furo[3,2-b]pyrrol-3-ol hydrochloride NEt₃, DCM, 40-45° C., (c) Dess-Martin periodinane, DCM.

### Step 1

1,1′-Carbonyldiimidazole (114 mg, 0.705 mmol) in dichloromethane (1 mL) was added to 1-benzylcyclobutanol (ALCOHOL 12) (76 mg, 0.470 mmol). The mixture was heated at 40° C. for 17 hours then diluted with dichloromethane (10 mL). The solution was washed with water (7 mL) then dried (Na₂SO₄), filtered and reduced in vacuo to give 1-benzylcyclobutyl 1H-imidazole-1-carboxylate which was used without further purification.

### Step 2

A solution of 1-benzylcyclobutyl 1H-imidazole-1-carboxylate (assumed to be 0.282 mmol, prepared as above) in dichloromethane (1.5 mL) was added to (3R,3aR,6aR)-hexahydro-2H-furo[3,2-b]pyrrol-3-ol hydrochloride (see compound 51, WO02057270) (50 mg, 0.302 mmol) followed by triethylamine (46 μL, 0.332 mmol). The mixture was heated at 40° C. with stirring for 3 days then at 45° C. for 10 days then concentrated in vacuo. The residue was purified by short path flash chromatography over silica, eluting with ethyl acetate:heptane mixtures 0:100 to 100:0 to give (3R,3aR,6aR)-1-benzylcyclobutyl 3-hydroxytetrahydro-2H-furo[3,2-b]pyrrole-4(5H)-carboxylate as a colourless oil (41 mg). TLC (R_{f}=0.39, EtOAc:heptane 3:2), analytical HPLC single main peak, Rₜ=13.25 min., HPLC-MS 340.2 [M+Na]⁺.

### Step 3

(3R,3aR,6aR)-1-Benzylcyclobutyl 3-hydroxytetrahydro-2H-furo[3,2-b]pyrrole-4(5H)-carboxylate (41 mg, assumed to be 0.13 mmol) in dichloromethane (1 mL) was added to Dess-Martin periodinane (110 mg, 0.26 mmol) under an atmosphere of argon. The mixture was stirred for 18 hours then diluted with dichloromethane (10 mL). The mixture was then washed with a mixture of 0.5M sodium thiosulphate:saturated aqueous sodium hydrogen carbonate (1:1, 8 mL), then saturated aqueous sodium hydrogen carbonate (8 mL), then brine (8 mL), then water (8 mL), then dried (Na₂SO₄), filtered and reduced in vacuo. Preparative thin layer chromatography eluting with ethylacetate:heptane 3:2 gave (3aS,6aR)-1-benzylcyclobutyl 3-oxotetrahydro-2H-furo[3,2-b]pyrrole-4(5B)-carboxylate as a colourless oil (18 mg, 44%). TLC (R_{f}=0.47, EtOAc:heptane 1:1), analytical HPLC broad main peak Rₜ=13.01-15.32 min., HPLC-MS 172.1 [M-PhCH₂^{c}Bu+H]⁺, 316.2 [M+H]⁺, 338.2 [M+Na]⁺, 356.2 [M+H₂O+Na]⁺, 653.3 [2M+Na]⁺.

### Example 22

### (3aS,6aR)-1-Phenethylcyclobutyl 3-oxotetrahydro-2H-furo[3,2-b]pyrrole-4(5H)-carboxylate

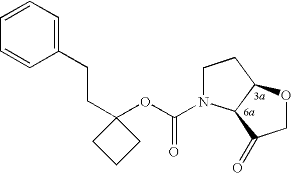

As detailed for EXAMPLE 21 but through use of 1-phenethylcyclobutanol (ALCOHOL 19).

TLC (R_{f}=0.50, EtOAc:heptane 1:1), analytical HPLC broad main peak, Rₜ=14.33-16.79 min., HPLC-MS 172.1 [M-Ph(CH₂)₂^{c}Bu+H]⁺, 352.2 [M+Na]⁺, 370.2 [M+H₂O+Na]⁺, 681.3 [2M+Na]⁺.

### Example 23

### (3aS,6aR)-1-(Thiophen-3-yl)butan-2-yl 3-oxotetrahydro-2H-furo[3,2-b]pyrrole-4(5H)-carboxylate

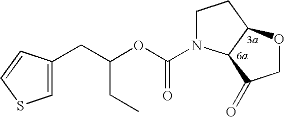

As detailed for EXAMPLE 21 but through use of 1-(thiophen-3-yl)butan-2-ol (ALCOHOL 40).

TLC (R_{f}=0.48, EtOAc:heptane 1:1), analytical HPLC broad main peak, Rₜ=11.77-14.92 min., HPLC-MS 310.1 [M+H]⁺, 350.1 [M+H₂O+Na]⁺, 641.3 [2M+Na]⁺.

### Example 24

### (3aS,6aR)-(1-(Phenoxymethyl)cyclobutyl)methyl 3-oxotetrahydro-2H-furo[3,2-b]pyrrole-4(5H)-carboxylate

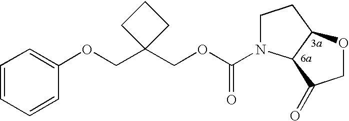

As detailed for EXAMPLE 21 but through use of (1-(phenoxymethyl)cyclobutyl)methanol (ALCOHOL 30) with Step 1 reaction time adjusted to 3 hours and Step 2 reaction time adjusted to 14 days at 40° C.

Analytical HPLC main peaks, Rₜ=16.21-19.03 min., HPLC-MS 346.2 [M+H]⁺, 368.1 [M+Na]⁺, 713.3 [2M+Na]⁺.

### Example 25

### (3aS,6aR)-1-(Thiophen-2-yl)butan-2-yl 3-oxotetrahydro-2H-furo[3,2-b]pyrrole-4(5H)-carboxylate

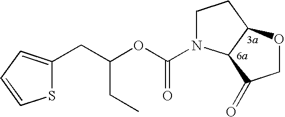

As detailed for EXAMPLE 21 but through use of 1-(thiophen-2-yl)butan-2-ol (ALCOHOL 42) with Step 1 reaction time adjusted to 15 hours and Step 2 reaction time adjusted to 13 days at 40° C.

Analytical HPLC broad main peak, Rₜ=14.51-13.67 min, HPLC-MS 310.1 [M+H]⁺, 350.1 [M+H₂O+Na]⁺, 641.2 [2M+Na]⁺.

### Example 26

### (3aS,6aR)-1-Isopropylcyclopropyl 3-oxotetrahydro-2H-furo[3,2-b]pyrrole-4(5H)-carboxylate

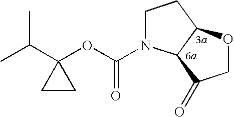

As detailed for EXAMPLE 21 but through use of 1-isopropylcyclopropanol (AlCOHOL 35) with Step 1 reaction time adjusted to 3 hours and Step 2 reaction time adjusted to 12 days at 40° C.

HPLC-MS two broad overlapping peaks, Rₜ=1.91 and 2.32 mins., 254.1 [M+H]⁺, 529.2 [2M+Na]⁺.

### Example 27

### (3aS,6aR)-5-Methyl-1-(thiophen-2-yl)hexan-3-yl 3-oxotetrahydro-2H-furo[3,2-b]pyrrole-4(5H)-carboxylate

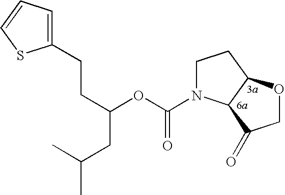

As detailed for EXAMPLE 21 but through use of 5-methyl-1-(thiophen-2-yl)hexan-3-ol (ALCOHOL 2) with Step 1 reaction time adjusted to 3 hours and Step 2 reaction time adjusted to 19 days at 40° C.

Analytical HPLC two main peaks, Rₜ=18.60 and 20.57 mm., HPLC-MS 352.2 [M+H₂O+H]⁺, 392.2 [M+Na]⁺, 725.3 [2M+Na]⁺.

### Example 28

### (3aS,6aR)-3-Methyl-1-phenylbutyl 3-oxotetrahydro-2H-furo[3,2-b]pyrrole-4(5H)-carboxylate

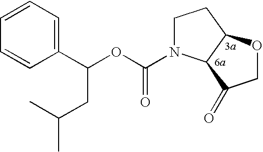

As detailed for EXAMPLE 21 but through use of 3-methyl-1-phenylbutan-1-ol (ALCOHOL 1) with Step 1 employing 2 mole equivalents of 1,1′-carbonyldiimidazole and reaction time adjusted to 2 days, and Step 2 reaction time adjusted to 5 days at 40° C.

Analytical HPLC main peaks, Rₜ=16.94-19.19 min., HPLC-MS 340.2 [M+Na]⁺, 358.2 [M+H₂O+Na]⁺, 657.3 [2M+Na]⁺, 693.3 [2(M+H₂O)+Na]⁺.

### Example 29

### (3aS,6aR)-5,5-Dimethylhexan-3-yl 3-oxotetrahydro-2H-furo[3,2-b]pyrrole-4(5H)-carboxylate

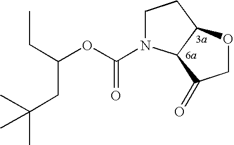

As detailed for EXAMPLE 21 but through use of 5,5-dimethylhexan-3-ol (ALCOHOL 28) with Step 1 reaction time adjusted to 2 hours and Step 2 reaction time adjusted to 19 days at 40° C. followed by microwave irradiation. (Catalyst model RG 31L, 700 W) for 40 minutes.

HPLC-MS two main peaks, Rₜ=2.72 and 3.08 mins., 284.2 [M+H]⁺, 324.2 [M+H₂O+Na]⁺, 589.3 [2M+Na]⁺, 625.3 [2(M+H₂O)+Na]⁺.

### Example 30

### (3aS,6aR)-4-Ethylbiphenyl-3-yl 3-oxotetrahydro-2H-furo[3,2-b]pyrrole-4(5H)-carboxylate

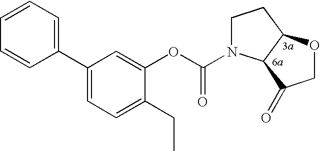

As detailed for EXAMPLE 21 but through use of 4-ethylbiphenyl-3-ol (ALCOHOL 29) with Step 1 reaction time adjusted to 2 hours and Step 2 reaction carried out by microwave irradiation. (Catalyst model RG 31L, 550 W) for 20 minutes.

Analytical HPLC broad main peak, Rₜ=14.90-17.52 min., HPLC-MS 352.2 [M+H]⁺, 725.3 [2M+Na]⁺.

### Preparation of Amines

1-(Thiophen-3-yl)butan-2-amine (AMINE 40)

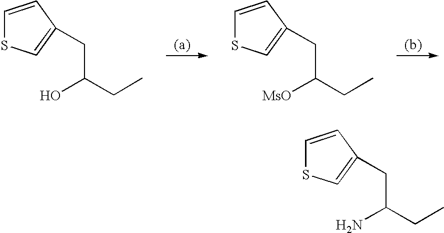

(a) MsCl, Et₃N, CH₂Cl₂; (b) NH₃, ⁱPrOH, H₂O, 60-C.

1-(Thiophen-3-yl)butan-2-yl methanesulfonate. Triethylamine (0.285 mL, 2.05 mmol) then methanesulfonyl chloride (0.149 mL, 1.92 mmol) were added consecutively to a stirred solution of 1-(thiophen-3-yl)butan-2-ol (ALCOHOL 40) (200 mg, 1.28 mmol) in dichloromethane (10 mL) under an atmosphere of argon. The solution was stirred for 2.25 hours then the solvents removed in vacuo. Flash chromatography over silica, eluting with ethyl acetate:pentane mixtures 0:100 to 25:75 gave 1-(thiophen-3-yl)butan-2-yl 0.75 methanesulfonate as a colourless oil (264 mg, 88%). TLC (R_{f}=0.30, EtOAc:heptane 1:4), analytical HPLC single main peak, Rₜ=12.91 min., HPLC-MS 139.1 [M-CH₃SO₃]⁺, 252.1 [M+H₂O]⁺, 257.0 [M+Na]⁺; δ_{H} (500, CDCl₃) 1.02 (3H, t, J=7.45 Hz, CH₂CH₃), 1.69-1.80 (2H, m, CH₂CH₃), 2.60 (3H, s, OSO₂CH₃), 3.00 (1H, brs, CH₂CHCH₂CH₃), 3.01 (1H, d J=2.52 Hz, CH₂CHCH₂CH₃), 4.74-4.80 (1H, m, CHCH₂), 6.99 (1H, dd, J=4.92 and 1.27 Hz, SCHCH), 7.07-7.09 (1H, m, CHCHSCH), 7.28 (1H, dd, J=4.92 and 2.96 Hz, SCHCO); δ_{C} (125 MHz, CDCl₃) 9.40 (CH₂CH₃), 27.77 (CH₂CH₃), 34.90 (CH₂CHCH₂CH₃), 37.82 (OSO₂CH₃), 85.45 (CHCOSO₂CH₃), 122.99/126.02 and 128.69 (heterocyclic CH), 136.96 (heterocyclic quaternary).

1-(Thiophen-3-yl)butan-2-amine. A stirred mixture of 1-(thiophen-3-yl)butan-2-yl methanesulfonate (50 mg, 0.21 mmol), ammonium hydroxide (1 mL) and ammonia in 2-propanol (2 mL, 2.0M, 4 mmol) was heated in a sealed tube at 60° C. for 26 hours. Similarly, a stirred mixture of 1-(thiophen-3-yl)butan-2-yl methanesulfonate (205 mg, 0.88 mmol), ammonium hydroxide (2 mL) and ammonia in 2-propanol (4 mL, 2.0M, a mmol) was heated in a sealed tube at 60° C. for 26 hours. The two reaction mixtures were combined then the solvents were removed in vacuo. The residue was azeotroped with toluene (2×2 mL) then the solvents removed in vacuo. Flash chromatography over silica, eluting with methanol:dichloromethane mixtures 2.5:97.5 to 10:90 gave 1-(thiophen-3-yl)butan-2-amine 0.75 methanesulfonate as a yellow oil (116 mg, 47%). TLC (R_{f}=0.10, methanol:dichloromethane 1:9), analytical HPLC single main peak, Rₜ=4.41 min., HPLC-MS 156.1 [M+H]⁺; δ_{H} (500 MHz, CD₃OD) 1.01 (3H, t, J=7.53 Hz, CH₂CH₃), 1.47-1.66 (2H, m, CH₂CH₃), 2.70 (2.25H, s, OSO₂CH₃), 2.79 (11H, dd, J=14.30 and 7.30 Hz, CH₂CHCH₂CH₃), 2.92 (1H, dd, J=14.29 and 6.30 Hz, CH₂CHCH₂CH₃), 3.17-3.23 (1H, m, CHN), 7.01 (1H, dd, J 4.94 and 1.24 Hz, SCHCH), 7.17-7.19 (1H, m, CHCHSCH), 7.40 (1H, dd, J 4.93 and 2.95 Hz, SCHCH); δ_{C} (125 MHz, CDCl₃) 9.74 (CH₂CH₃), 26.41 (CH₂CH₃), 33.81 (CH₂CHCH₂CH₃), 39.46 (OSO₂CH₃), 54.90 (CHNH₂), 124.36/127.76 and 129.21 (heterocyclic CH), 137.17 (heterocyclic quaternary).

### 1-(Thiophen-2-yl)butan-2-amine (AMINE 42)

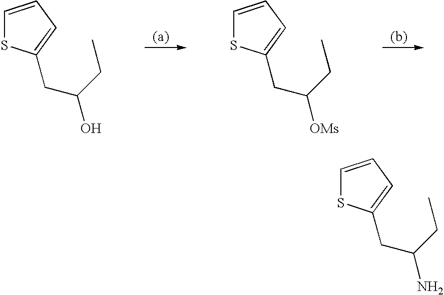

(a) MsCl, Et₃N, CH₂Cl₂; (b) NH₃, ⁱPrOH, H₂O, 60° C.

1-(Thiophen-2-yl)butan-2-yl methanesulfonate. Triethylamine (0.285 mL, 2.05 mmol) then methanesulfonyl chloride (0.149 mL, 1.92 mmol) were added consecutively to a stirred solution of 1-(thiophen-2-yl)butan-2-ol (ALCOHOL 42) (200 mg, 1.28 mmol) in dichloromethane (10 mL) under an atmosphere of argon. The solution was stirred for 3 hours then the solvents removed in vacuo. Flash chromatography over silica, eluting with ethyl acetate:pentane mixtures 0:100 to 20:80 gave 1-(thiophen-2-yl)butan-2-yl methanesulfonate as a colourless oil (261 mg, 88%). TLC (R_{f}=0.35, EtOAc: heptane 1:3), analytical HPLC single main peak, Rₜ=13.35 mill., HPLC-MS 139.1 [M CH₃SO₃]⁺, 252.1 [M+H₂O]⁺, 257.0 [M+Na]⁺; OH (500 MHz, CDCl₃) 1.02 (3H, t, J=7.45 Hz, CH₂CH₃), 1.72-1.84 (2H, m, CH₂CH₃), 2.69 (3H, S, OSO₂CH₃), 3.19 (2H, d, J=6.21 Hz, CH₂CHCH₂CH₃), 4.74-4.80 (1H, m, CHCH₂), 6.89 (1H, dd, J=3.41 and 0.88 Hz, SCHCHCH), 6.95 (1H, dd, J=5.12 and 3.44 Hz, SCHCHCH), 7.18 (1H, dd, J=5.13 and 1.18 Hz, SCHCHCH).

1-(Thiophen-2-yl)butan-2-amine. A stirred mixture of 1-(thiophen-2-yl)butan-2-yl methanesulfonate (253 mg, 1.08 mmol), ammonium hydroxide (3 mL) and ammonia in 2-propanol (6 mL, 2.0M, 12 mmol) was heated in a sealed tube at 60° C. for 24 hours then the solvents were removed in vacuo. The residue was azeotroped with toluene (2×2 mL) then the solvents removed in vacuo. The residue was stirred and heated at 60° C. in a sealed tube for 24 hours with ammonia in 2-propanol (6 mL, 2.0M, 12 mmol) then ammonium hydroxide (3 mL) added and heating continued at 60° C. in a sealed tube for 24 hours. The solvents were then removed in vacuo. Flash chromatography over silica, eluting with methanol:dichloromethane mixtures 1.5:98.5 to 10:90 gave 1-(thiophen-2-yl)butan-2-amine 0.8 methanesulfonate as a yellow oil (135 mg, 54%). TLC (R_{f}=0.15, methanol:dichloromethane 1:9), analytical HPLC single main peak, Rₜ=4.35 min., HPLC-MS 156.1 [M+H]⁺; δ_{C} (500 MHz, CD₃OD) 1.01 (3H, t, J=7.53 Hz, CH₂CH₃), 1.52-1.70 (2H, m, CH₂CH₃), 2.69 (2.4H, S, OSO₂CH₃), 2.99 (1H, dd, J=14.84 and 7.12 Hz, CH₂CHCH₂CH₃), 3.12 (1H, dd, J=14.84 and 6.19 Hz, CH₂CHCH₂CH₃), 3.15-3.23 (1H, m, CHN), 6.93 (1H, dd, J=3.41 and 0.88 Hz, SCHCHCB), 6.99 (1H, dd, J=5.15 and 3.46 Hz, SCHCHCH), 7.30 (1H, dd, J=5.16 and 1.14 Hz, SCHCHCH).

### (1-(Phenoxymethyl)cyclobutyl)methanamine (AMINE 309

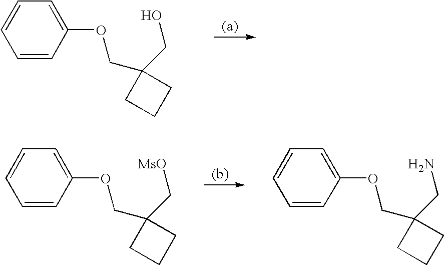

(a) MsCl, Et₃N, CH₂Cl₂; (b) NH₃, ⁱPrOH, H₂O, 60° C.

(1-(Phenoxymethyl)cyclobutyl)methyl methanesulfonate. Triethylamine (0.174 mL, 1.25 mmol) then methanesulfonyl chloride (0.091 mL, 1.17 mmol) were added consecutively to a stirred solution of (1-(phenoxymethyl)cyclobutyl)methanol (ALCOHOL 30) (150 mg, 0.78 mmol) in dichloromethane (7.5 mL) under an atmosphere of argon. The solution was stirred for 3 hours then the solvents removed in vacuo. Flash chromatography over silica, eluting with ethyl acetate:pentane mixtures 0:100 to 20:80 gave (1-(phenoxymethyl)cyclobutyl)methyl methanesulfonate as a colourless oil (201 mg, 95%). TLC (R_{f}=0.25, EtOAc:heptane 1:3), analytical HPLC single main peak, Rₜ=16.43 min., HPLC-MS 175.1 [M-CH₃SO₃]⁺, 288.1 [M+H₂O]⁺, 293.1 [M+Na]⁺; δ_{H} (500, CDCl₃) 2.00 (6H, s, CH₂CH₂CH₂), 2.94 (3H, s, OSO₂CH₃), 3.97 (2H, s, CH₂OS), 4.36 (2H, m, PhOCH₂), 6.88-6.93 (3H, m, aromatic CH), 7.26-7.30 (2H, m, aromatic CH).

(1-(Phenoxymethyl)cyclobutyl)methanamine. A stirred mixture of (1-(phenoxymethyl)cyclobutyl)methyl methanesulfonate (195 mg, 0.72 mmol), ammonium hydroxide (2.5 mL) and ammonia in 2-propanol (5 mL, 2.0M, 10 mmol) was heated in a sealed tube at 60° C. for 48 hours then the solvents were removed in vacuo. Flash chromatography over silica, eluting with methanol:dichloromethane mixtures 1.5:98.5 to 10:90 gave (1-(phenoxymethyl)cyclobutyl)methanamine hemimethanesulfonate as a colourless oil (92 mg, 53%). TLC (R_{f}=0.10, methanol dichloromethane 1:9), analytical HPLC single main peak, Rₜ=8.59 min., HPLC-MS 192.2 [M+H]⁺; 5H (500 MHz, CD₃OD) 1.92-2.06 (6H, m, CH₂CH₂CH₂), 2.69 (1.5H, s, OSO₂CH₃), 3.00 (2H, s, CH₂NH₂), 4.03 (2H, m, CH₂OPh), 6.91-6.97 (3H, m, aromatic CB), 7.24-7.29 (2H, m, aromatic CH).

### 5-Methyl-1-(thiophen-2-yl)hexan-3-amine (AMINE 2)

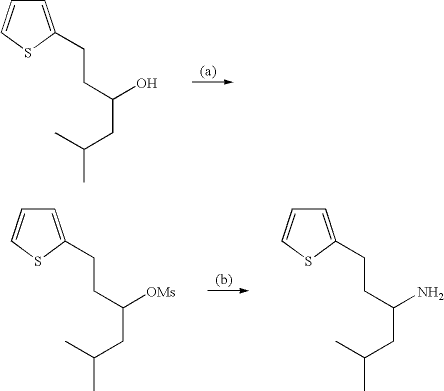

(b) MsCl, Et₃N, CH₂Cl₂; (b) NH₃, ⁱPrOH, H₂O, 60° C.

5-Methyl-1-(thiophen-2-yl)hexan-3-yl methanesulfonate. Triethylamine (0.101 mL, 0.73 mmol) then methanesulfonyl chloride (0.053 mL, 0.68 mmol) were added consecutively to a stirred solution of 5-methyl-1-(thiophen-2-yl)hexan-3-ol (ALCOHOL 2) (90 mg, 0.45 mmol) in dichloromethane (4.5 mL) under an atmosphere of argon. The solution was stirred for 3.75 hours then the solvents removed in vacuo. Flash chromatography over silica, eluting with ethyl acetate:pentane mixtures 0:100 to 20:80 gave 5-methyl-1-(thiophen-2-yl)hexan-3-yl methanesulfonate as a colourless oil (100 mg, 80%). TLC (R_{f}=0.30, EtOAc:heptane 1:4), analytical HPLC single main peak, Rₜ=17.93 min., HPLC-MS 181.1 [M-CH₃SO₃]⁺, 294.1 [+H₂O]⁺, 299.1 [M+Na]⁺; 5H (500 MHz, CDCl₃) 0.92 (3H, d, J=6.34 Hz, CHCH₃), 0.94 (3H, d, J=6.42 Hz, CHCH₃), 1.44-1.52 (1H, m, CHCH₃), 1.68-1.76 (2H, m, CHCH₂CHO), 2.07 (2H, dt, J=5.83 and 7.91 Hz, CH₂CH₂CHO), 2.93-3.00 (2H, m, CH₂CH₂CHO), 3.00 (3H, s, OSO₂CH₃), 4.82-4.87 (1H, m, CHO), 6.82-6.84 (1H, m, SCHCHCH), 6.91 (1H, dd, J=5.13 and 3.42 Hz, SCHCHCH), 7.12 (1H, dd, J=5.13 and 1.18 Hz, SCHCHCH).

5-Methyl-1-(thiophen-2-yl)hexan-3-amine. A stirred mixture of 5-methyl-1-(thiophen-2-yl)hexan-3-yl methanesulfonate (95 mg, 0.34 mmol), ammonium hydroxide (1.5 mL) and ammonia in 2-propanol (3 mL, 2.0M, 6 mmol) was heated in a sealed tube at 60° C. for 30.5 hours then the solvents were removed in vacuo. Flash chromatography over silica, eluting with methanol:dichloromethane mixtures 2.5:97.5 to 10:90 gave 5-methyl-1-(thiophen-2-yl)hexan-3-amine hemimethanesulfonate as a yellow oil (35 mg, 41%). TLC (R_{f}=0.10, methanol:dichloromethane 1:9), analytical HPLC single main peak, Rₜ=10.52 min., HPLC-MS 198.1 [M+H]⁺; 8H (500 M CD₃OD) 0.91 (3H, d, J=2.07 Hz, CHCH₃), 0.92 (3H, d, J=2.11 Hz, CHCH₃), 1.32-1.43 (2H, n, CHCH₂CHN and 1×CHCH₂CHN), 1.67-1.91 (3H, m, CH₂CH₂CHN and 1×CHCH₂CHN), 2.69 (1.5H, s, OSO₂CH₃), 2.88-3.04 (3H, m, CH₂CH₂CHN), 6.85 (1H, dd, J=3.41 and 1.01 Hz, SCHCHCH), 6.91 (1H, dd, J=5.14 and 3.42 Hz, SCHCHCH), 7.19 (1H, dd, J=5.15 and 1.17 Hz, SCHCHCH).

### Preparation of Isocyanates

3-(2-Isocyanatobutyl)thiophene (ISOCYANATE 40)

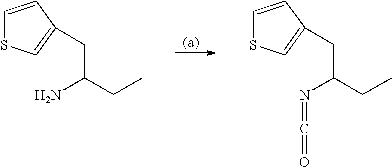

(a) COCl₂, toluene, THF.

A solution of phosgene in toluene (20%, 1.31 mL, 2.49 mmol) was added to a solution of 1-(thiophen-3-yl)butan-2-amine 0.75 methanesulfonate (AMINE 40) (113 mg, 0.498 mmol) in tetrahydrofuran (2 mL). The mixture was stirred for 24 hours then solvents were removed in vacuo. The residue was azeotroped with toluene (2 mL) to obtain 3-(2-isocyanatobutyl)thiophene which was used without further purification.

HPLC-MS of morpholine adduct Rₜ=1.98 nm in, 269.2 [M+H]⁺, 559.2 [M+Na]⁺.

### 2-(2-Isocyanatobutyl)thiophene (ISOCYANATE 42)

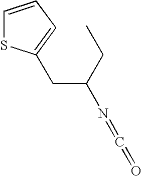

Prepared as detailed for ISOCYANATIE 40 but through use of 1-(thiophen-2-yl)butan-2-amine 0.8 methanesulfonate (AMINE 42).

HPLC-MS of morpholine adduct Rₜ=1.98 ml, 269.2 [M+H]⁺, 559.2 [M+Na]⁺.

### ((1-(Isocyanatomethyl)cyclobutyl)methoxy)benzene (ISOCYANATE 30)

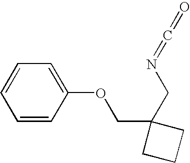

Prepared as detailed for ISOCYANATE 40 but through use of (1-(phenoxymethyl)cyclobutyl)methanamine hemimethanesulfonate (AMINE 30).

HPLC-MS of morpholine adduct Rₜ=2.66 min, 305.2 [M+H]⁺, 631.4 [M+Na]⁺.

### 1-Chloro-3-fluoro-2-(isocyanatomethyl)-4-methylbenzene (ISOCYANATE 43)

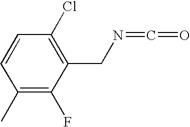

Prepared as detailed for ISOCYANATE 40 but through use of (6-chloro-2-fluoro-3-methylphenyl)methanamine (ex Fluorochem).

HPLC-MS of morpholine adduct Rₜ=2.15 min, 287.1 [M+H]⁺, 595.2 [M+Na]⁺.

### 2-(3-Isocyanato-5-methylhexyl)thiophene (ISOCYANATE 2)

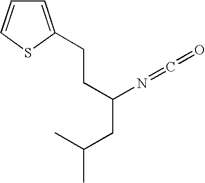

A solution of phosgene in toluene (20%, 0.366 mL, 0.695 mmol) was added to a solution of 5-methyl-1-(thiophen-2-yl)hexan-3-amine hemimethanesulfonate (AMINE 2) (34 mg, 0.139 mmol) in tetrahydrofuran (0.5 mL). The mixture was stirred for 24 hours then a solution of phosgene in toluene (20%, 0.366 mL, 0.695 mmol) was added. The mixture was sired for 24 hours then solvents were removed in vacuo to obtain 2-(3-isocyanato-5-methylhexyl)thiophene which was used without further purification. HPLC-MS of morpholine adduct Rₜ=2.87 min, 311.2 [M+H]⁺, 643.3 [M+Na]⁺.

### Example 31

### (3aR,6aS)-4-Benzoyl-6-oxo-N-(1-(thiophen-3-yl)butan-2-yl)hexahydropyrrolo[3,2-b]pyrrole-1(2H)-carboxamide

Prepared following the general solid phase protocols through use of ISOCYANATE 40.

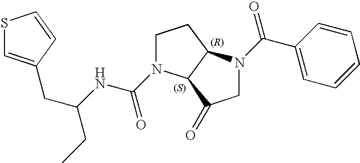

Analytical HPLC main peaks Rₜ=14.06-15.32 min, HPLC-MS, 412.2 [M+H]⁺, 845.2 [M+Na]⁺.

### Example 32

### (3aR,6aS)-6-Oxo-4-(pyrrolidine-1-carbonyl)-N-(1-(thiophen-3-yl)butan-2-yl)hexahydropyrrolo[3,2-b]pyrrole-1(2H)-carboxamide

Prepared following the general solid phase protocols through use of ISOCYANATE 40.

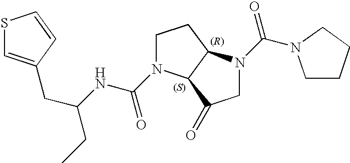

Analytical HPLC two main peaks Rₜ=13.85 and 14.38 min, HPLC-MS, 405.2 [M+H]⁺, 427.2 [M+Na]⁺, 831.2 [2M+Na]⁺.

### Example 33

### (3aR,6aS)-4-Benzoyl-6-oxo-N-(1-(thiophen-2-yl)butan-2-yl)hexahydropyrrolo[3,2-b]pyrrole-1(2H)-carboxamide

Prepared following the general solid phase protocols through use of ISOCYANATE 42.

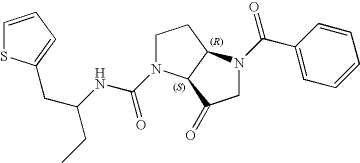

Analytical HPLC two main peaks Rₜ=14.91 and 15.55 min. HPLC-MS, 412.1 [M+H]⁺, 845.2 [2M+Na]⁺.

### Example 34

### (3aR,6aS)-6-Oxo-4-(pyrrolidine-1-carbonyl)-N-(1-(thiophen-2-yl)butan-2-yl)hexahydropyrrolo[3,2-b]pyrrole-1(2H)-carboxamide

Prepared following the general solid phase protocols through use of ISOCYANATE 42.

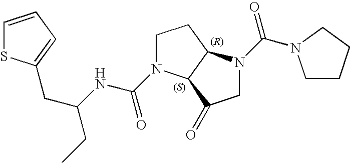

Analytical HPLC two main peaks Rₜ=13.97 and 14.42 min, HPLC-MS, 405.2 [M+H]⁺, 831.3 [2M+Na]⁺.

### Example 35

### (3aR,6aS)-4-Benzoyl-6-oxo-N-((1-(phenoxymethyl cyclobutyl)methyl)hexahydropyrrolo[3,2-b]pyrrole-1(2H)-carboxamide

Prepared following the general solid phase protocols through use of ISOCYANATE 30.

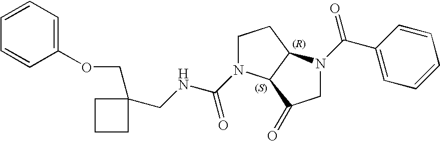

Analytical HPLC main peak Rₜ=17.72 min, HPLC-MS, 448.2 [M+H]⁺.

### Example 36

### (3aR,6aS)-4-Benzoyl-N-(5-methyl-1-(thiophen-2-yl)hexan-3-yl)-6-oxohexahydro pyrrolo[3,2-b]pyrrole-1(2H)-carboxamide

Prepared following the general solid phase protocols through use of ISOCYANATE 2.

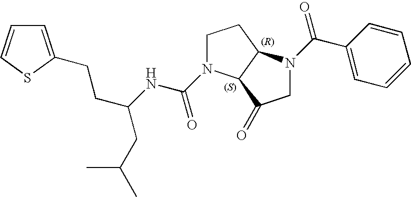

Analytical HPLC two main peaks Rₜ=18.50 and 18.81 min, HPLC-MS, 454.2 [M+H]⁺, 907.3 [2M+H]⁺.

### Example 37

### (3aR,6aS)-4-Benzoyl-N-(6-chloro-2-fluoro-3-methylbenzyl)-6-oxohexahydro pyrrolo[3,2-b]pyrrole-1(2H)-carboxamide

Prepared following the general solid phase protocols through use of ISOCYANATE 43.

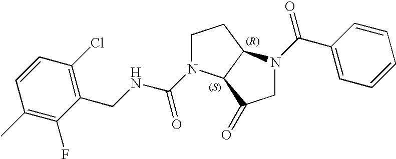

Analytical HPLC single main peak Rₜ=14.64 min, HPLC-MS, 430.1/432.1 [M+H]⁺, 881.1/885.1 [2M+Na]⁺.

### Example 38

### (3aR,6aS)—N-(6-Chloro-2-fluoro-3-methylbenzyl)-6-oxo-4-(pyrrolidine-1-carbonyl-hexahydropyrrolo[3,2-b]pyrrole-1(2H)-carboxamide

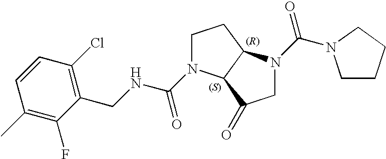

Analytical HPLC single main peak Rₜ=13.09 min, HPLC-MS, 423.1/425.1 [M+H]⁺, 867.1/871.2 [2M+Na]⁺.

### Example 39

### (3aR,6aS-4-Benzoyl-N-(biphenyl-2-yl)-6-oxohexahydropyrrolo[3,2-b]pyrrole-1(2H)-carboxamide

Prepared following the general solid phase protocols through use of 2-isocyanatobiphenyl (ex Maybridge).

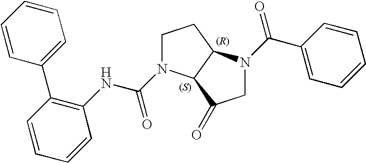

Analytical HPLC single main peak Rₜ=14.75 min, HPLC-MS, 426.2 [M+H]⁺, 851.2 [2M+H]⁺, 873.2 [2M+Na]⁺.

### Example 40

### (3aR,6aS)-4-Benzoyl-N-(2-ethoxyphenyl)-6-oxohexahydropyrrolo[3,2-b]pyrrole-1(2H)-carboxamide

Prepared following the general solid phase protocols through use of 1-ethoxy-2-isocyanatobenzene (ex Lancaster).

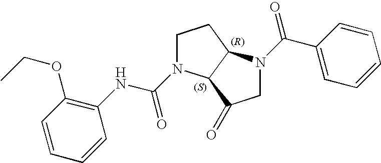

Analytical HPLC single main peak Rₜ=12.23 min, HPLC-MS, 394.2 [M+H]⁺, 809.2 [2M+Na]⁺.

### Example 41

### (3aR,6aS)-4-Benzoyl-6-oxo N-(2-propylphenyl)hexahydropyrrolo[3,2-b]pyrrole-1(2H)-carboxamide

Prepared following the general solid phase protocols through use of 1-isocyanato-2-propylbenzene (ex Aldrich).

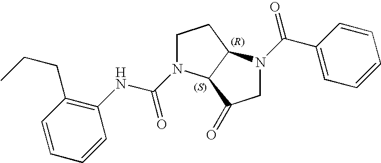

Analytical HPLC single main peak Rₜ=14.67 min, HPLC-MS, 392.2 [M+H]⁺, 783.3 [2M+H]⁺.

### Example 42

### (3a,6aS)-4-Benzoyl-N-(2-chloro-5-(trifluoromethyl)phenyl)-6-oxohexahydropyrrolo[3,2-b]pyrrole-1(2H)-carboxamide

Prepared following the general solid phase protocols through use of 1-chloro-2-isocyanato-4-(trifluoromethyl)benzene (ex Lancaster).

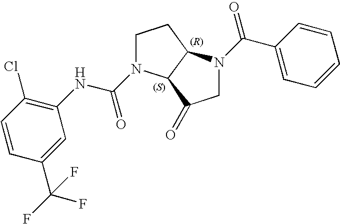

Analytical HPLC single main peak Rₜ=15.43 min, HPLC-MS, 452.1/454.1 [M+H]⁺, 925.0/927.0 [2M+Na]⁺.

### Example 43

### (3aS,6aR)-3-Oxo-N-(1-(thiophen-3-yl)butan-2-yl)tetrahydro-2H-furo[3,2-b]pyrrole-4(5H)carboxamide

Prepared following the general solid phase protocols through use of ISOCYANATE 40.

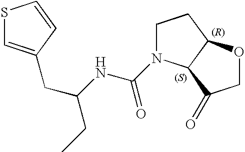

Analytical HPLC two main peaks Rₜ=12.08 and 12.55 min, HPLC-MS, 309.1 [M+H]⁺, 331.1 [M+Na]⁺, 639.2 [2M+Na]⁺.

### Example 44

### (3aS,6aR)-3-Oxo-N-(1-(thiophen-2-yl)butan-2-yl)tetrahydro-2H-furo[3,2-b]pyrrole-4(5H)-carboxamide

Prepared following the general solid phase protocols through use of ISOCYANATE 42.

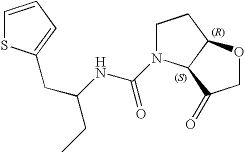

Analytical HPLC two main peaks Rₜ=12.23 and 12.55 min, HPLC-MS, 309.1 [M+H]⁺, 331.1[M+Na]⁺, 639.2 [2M+Na]⁺.

### Example 45

### (3aS,6aR)-3-Oxo-N-((1-(phenoxymethyl)cyclobutyl)methyl)tetrahydro-2H-furo[3,2-b]pyrrole-4(5H)-carboxamide

Prepared following the general solid phase protocols through use of ISOCYANATE 30.

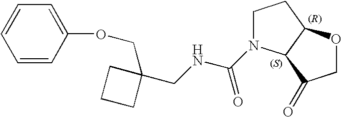

Analytical HPLC single main peak Rₜ=15.80 min, HPLC-MS, 345.2 [M+H]⁺, 711.2 [2M+Na]⁺.

### Example 46

### (3aS,6aR)—N-(5-Methyl-1-(thiophen-2-yl)hexan-3-yl)-3-oxotetrahydro-2H-furo[3,2-b]pyrrole-4(5H)-carboxamide

Prepared following the general solid phase protocols through use of ISOCYANATE 2.

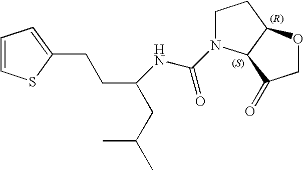

Analytical HPLC two main peaks Rₜ=16.99 and 17.23 min, HPLC-MS, 351.2 [M+H]⁺, 373.2 [M+Na]⁺, 701.2 [2M+H]⁺, 723.2 [2M+Na]⁺.

### Example 47

### (3aS,6aR)—N-(6-Chloro-2-fluoro-3-methylbenzyl)-3-oxotetrahydro-2H-furo[3,2-b]pyrrole-4(5H)-carboxamide

Prepared following the general solid phase protocols through use of ISOCYANATE 43.

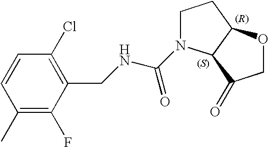

Analytical HPLC single main peak Rₜ=12.14 min, HPLC-MS, 327.1/329.1 [M+H]⁺, 675.1/679.1 [2M+Na]⁺.

### Example 48

### (3aS,6aR)—N-(Biphenyl-2-yl)-3-oxotetrahydro-2H-furo[3,2-b]pyrrole-4(5H)carboxamide

Prepared following the general solid phase protocols through use of 2-isocyanatobiphenyl (ex Maybridge).

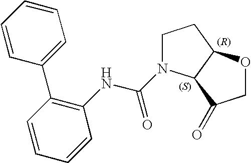

Analytical HPLC single main peak Rₜ=11.85 min, HPLC-MS, 323.2 [M+H]⁺, 667.2 [2M+Na]⁺.

### Example 49

### (3aS,6aR)—N-(2-Ethoxyphenyl)-3-oxotetrahydro-2H-furo[3,2-b]pyrrole-4(5H)-carboxamide

Prepared following the general solid phase protocols through use of 1-ethoxy-2-isocyanatobenzene (ex Lancaster).

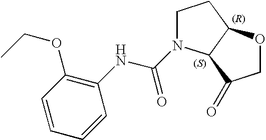

Analytical HPLC single main peak Rₜ=8.50 min, HPLC-MS, 291.1 [M+H]⁺, 603.2 [2M+Na]⁺.

### Example 50

### (3aS,6aR)-3-Oxo-N-(2-propylphenyl)tetrahydro-2H-furo[3,2-b]pyrrole-4(5H)-carboxamide

Prepared following the general solid phase protocols through use of 1-isocyanato-2-propylbenzene (ex Aldrich).

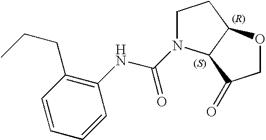

Analytical HPLC single main peak Rₜ=11.86 min, HPLC-MS, 289.2 [M+H]⁺, 577.2 [2M+H]⁺, 599.2 [2M+Na]⁺.

### Example 51

### (3aS,6aR)—N-(2-Chloro-5-(trifluoromethyl)phenyl)-3-oxotetrahydro-2H-furo[3,2-b]pyrrole-4(5H)-carboxamide

Prepared following the general solid phase protocols through use of 1-chloro-2-isocyanato-4-(trifluoromethyl)benzene (ex Lancaster).

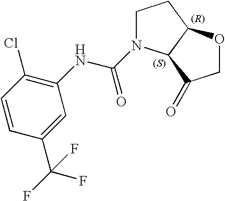

Analytical HPLC single main peak Rₜ=12.45 min, HPLC-MS, 349.0/351.1 [M+H]⁺, 697.0/699.0 [2M+H]⁺, 719.01721.0 [2M+Na]⁺.

### Example 52

### (3aR,6aS)—S-6-Chloro-2-fluoro-3-methylbenzyl 4-benzoyl-6-oxohexahydropyrrolo[3,2-b]pyrrole-1(2H)-carbothioate

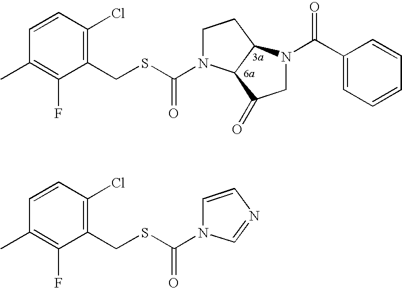

(i) S-(6-Chloro-2-fluoro-3-methylphenyl)methanethiol. 6-Chloro-2-fluoro-3-methylbenzylalcohol (200 mg, 1.15 mmol) and thiourea (92 mg, 1.20 mmol) were suspended in aqueous hydrobromic acid (48% v/v, 1.5 mL) and the mixture heated at 95° C. with stirring for 24 hours then the solvents were removed in vacuo. The resulting white solid was dissolved in water (4 mL) and 3M sodium hydroxide (0.67 mL, 2.30 mmol) added then the reaction was stirred for 18 hours. The mixture was acidified to pH=1 with 1M hydrochloric acid and stirred for 2 hours. The reaction was diluted with water (7 mL), then extracted with dichloromethane (2×10 mL). The organic layer was dried (Na₂SO₄), filtered and reduced in vacuo to give S-(6-chloro-2-fluoro-3-methylphenyl)methanethiol as a pale yellow oil (190 mg) which was used without further purification.

(ii) S-6-Chloro-2-fluoro-3-methylbenzyl 1H-imidazole-1-carbothioate. Carbonyl diimidazole (242 mg, 1.50 mmol) in dichloromethane (0.5 mL) was added to S-(6-chloro-2-fluoro-3-methylphenyl)methanethiol (190 mg, assumed to be 1.00 mmol, prepared as above). The mixture was stirred for 1 hour then allowed to stand for 18 hours. The mixture was diluted with dichloromethane (10 mL), then washed with water (10 mL), then dried (Na₂SO₄), filtered and reduced in vacuo to give S-6-chloro-2-fluoro-3-methylbenzyl 1H-imidazole-1-carbothioate (280 mg) which was used without further purification.

(iii) (3aR,6S,6aS)—S-6-Chloro-2-fluoro-3-methylbenzyl 4-benzoyl-6-hydroxyhexahydropyrrolo[3,2-b]pyrrole-1(2H)-carbothioate. S-6-Chloro-2-fluoro-3-methylbenzyl 1H-imidazole-1-carbothioate (49 mg, assumed to be 0.17 mmol, prepared as above) in dichloromethane (1 mL) was added to ((3S,3aS,6aR)-3-hydroxyhexahydropyrrolo[3,2-b]pyrrol-1(2H)-yl)(phenyl)methanone (40 mg, 0.17 mmol). The mixture was stirred for 3 days then heated at 40° C. with stirring for 1 day. S-6-Chloro-2-fluoro-3-methylbenzyl 1H-imidazole-1-carbothioate (10 mg, assumed to be 0.04 mmol, prepared as above) was added and the mixture heated at 40° C. for 1 day then the solvents were removed in vacuo. Short path flash chromatography over silica, eluting with ethyl acetate heptane mixtures 0:100 to 100:0 gave (3aR,6S,6aS)—S-6-chloro-2-fluoro-3-methylbenzyl 4-benzoyl-6-hydroxyhexahydropyrrolo[3,2-b]pyrrole-1(2H)-carbothioate as a pale yellow oil (50 mg). TLC (R_{f}=0.34, EtOAc:heptane 3:2), analytical HPLC single main peak, Rₜ=16.51 min., HPLC-MS 449.1 [M+H]⁺, 919.1 [2M+Na]⁺.

(iv) (3aR,6aS)—S-6-Chloro-2-fluoro-3-methylbenzyl 4-benzoyl-6-oxohexahydropyrrolo[3,2-b]pyrrole-1(2H)-carbothioate. A solution of (3aR,6S,6aS)—S-6-chloro-2-fluoro-3-methylbenzyl 4-benzoyl-6-hydroxyhexahydropyrrolo[3,2-b]pyrrole-1(2H)-carbothioate (50 mg, 0.11 mmol) in dichloromethane (1 mL) was added to Dess-Martin periodinane (94 mg, 0.22 mmol) under an atmosphere of argon. The mixture was stirred for 18 hours then diluted with dichloromethane (12 mL). The organic phase was washed with a mixture of 0.5M sodium thiosulphate:saturated aqueous sodium hydrogen carbonate (1:1, 8 mL), then saturated aqueous sodium hydrogen carbonate (8 mL), then brine (8 mL), then water (8 mL), then dried (Na₂SO₄), filtered and reduced in vacuo. Flash chromatography over silica, eluting with ethyl acetate:heptane mixtures 5:95 to 50:50 gave (3aR,6aS)—S-6-chloro-2-fluoro-3-methylbenzyl 4-benzoyl-6-oxohexahydropyrrolo[3,2-b]pyrrole-1(2H)-carbothioate as a white solid (5.81 mg, 8%). TLC (R_{f}=0.23, EtOAc:heptane 1:1), analytical HPLC broad main peak, Rₜ=16.040-17.36 min., HPLC-MS 447.1 [M+H]⁺, 465.1 [M+H₂O+H]⁺, 915.0 [2M+Na]⁺.

### Example 53

### (3R,3aR,6aR)—S-6-chloro-2-fluoro-3-methylbenzyl 3-hydroxytetrahydro-2H-furo[3,2-b]pyrrole-4(5H)-carbothioate

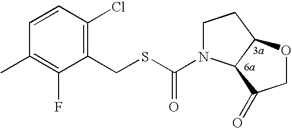

(i) (3R,3aR,6aR)—S-6-Chloro-2-fluoro-3-methylbenzyl. 3-hydroxytetrahydro-2H-furo[3,2-b]pyrrole-4(5H)-carbothioate. S-6-Chloro-2-fluoro-3-methylbenzyl 1H-imidazole-1-carbothioate (86 mg, assumed to be 0.30 mmol, prepared as above) in dichloromethane (1 mL) was added to (3R,3aR,6aR)-hexahydro-2H-furo[3,2-b]pyrrol-3-ol hydrochloride (50 mg, 0.30 mmol). The mixture was heated at 40° C. with stirring for 3 days then S-6-chloro-2-fluoro-3-methylbenzyl 1H-imidazole-1-carbothioate (30 mg, assumed to be 0.11 mmol, prepared as above) was added and the mixture heated at 40° C. for 1 day then the solvents were removed in vacuo. Short path flash chromatography over silica, eluting with ethyl acetate:heptane mixtures 0:100 to 100:0 gave (3R,3aR,6aR)—S-6-chloro-2-fluoro-3-methylbenzyl 3-hydroxytetrahydro-2H-furo[3,2-b]pyrrole-4(5H)-carbothioate as a pale yellow oil (28 mg). Analytical HPLC broad main peak, Rₜ=16.13-17.48 min., HPLC-MS 346.1 [M+H]⁺, 713.1 [2M+Na]⁺.

(ii) (3aS,6aR)—S-6-Chloro-2-fluoro-3-methylbenzyl 3-oxotetrahydro-2H-furo[3,2-b]pyrrole-4(5H)-carbothioate. A solution of (3R,3aR,6aR)—6-chloro-2-fluoro-3-methylbenzyl-3-hydroxytetrahydro-2H-furo[3,2-b]pyrrol-4(5H)-carbothioate (28 mg, 0.11 mmol) in dichloromethane (1 mL) was added to Dess-Martin periodinane (97 mg, 0.23 mmol) under an atmosphere of argon. The mixture was stirred for 18 hours then diluted with dichloromethane (12 mL). The organic phase was washed with a mixture of 0.5M sodium thiosulphate:saturated aqueous sodium hydrogen carbonate (1:1, 8 mL), then saturated aqueous sodium hydrogen carbonate (8 mL), then brine (8 mL), then water (8 mL), then dried Na₂SO₄), filtered and reduced in vacuo. Flash chromatography over silica, eluting with ethyl acetate:heptane mixtures 5:95 to 50:50 gave (3aS,6aR)—S-6-chloro-2-fluoro-3-methylbenzyl 3-oxotetrahydro-2H-furo[3,2-b]pyrrole-4(5H)-carbothioate as a pale yellow oil (10 mg, 26%). TLC (R_{f}=0.42, EtOAc:heptane 1:1), analytical HPLC broad main peak, Rₜ=13.86-16.32 ml. HPLC-MS 344.1 [M+H]⁺, 709.0 [2M+Na]⁺.

### Example 54

### (3aR,6aS-2-Ethoxy-4-methylphenyl 4-benzoyl-6-oxohexahydropyrrolo[3,2-b]pyrrole-1(2H)-carboxylate

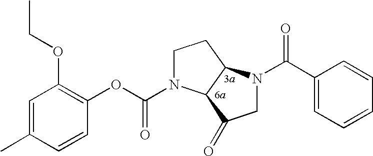

(i) (3aR,6S,6aS)-2-Ethoxy-4-methylphenyl 4-benzoyl-6-hydroxyhexahydropyrrolo[3,2-b]pyrrole-1(2H)-carboxylate. 1,1′-Carbonyldiimidazole (30 mg, 0.17 mmol) in dichloromethane (0.5 mL) was added to a stirred solution of 2-ethoxy-4-methylphenol (22 mg, 0.14 mmol) in dichloromethane (0.5 mL). The mixture was heated at 40° C. for 2.5 hours then diluted with dichloromethane (10 mL). The solution was washed with water (7 mL) then dried (Na₂SO₄), filtered and reduced in vacuo to give 2-ethoxy-4-methylphenyl 1H-imidazole-1-carboxylate which was used without further purification.

A solution of 2-ethoxy-4-methylphenyl 1H-imidazole-1-carboxylate (assumed to be 0.14 mmol, prepared as above) in tetrahydrofuran (0.5 mL) was added to ((3S,3aS,6aR)-3-hydroxyhexahydropyrrolo[3,2-b]pyrrol-1(2H)-yl)(phenyl)methanone (40 mg, 0.17 mmol) in tetrahydrofuran (0.5 mL). The mixture was heated at 65° C. with stirring for 3 days then concentrated in vacuo. The residue was purified by short path chromatography using an ISOLUTE SPE PE-AX/SCX-2 cartridge eluting with methanol:dichloromethane mixtures 0:100 to 10:90 followed by short path flash chromatography over silica, eluting with ethyl acetate:heptane mixtures 0:100 to 100:0 to give (3aR,6S,6aS)-2-ethoxy-4-methylphenyl 4-benzoyl-6-hydroxyhexahydropyrrolo[3,2-b]pyrrole-1(2H)-carboxylate as a white solid (27 mg). TLC (R_{f}=0.07, EtOAc:heptane 1:1), HPLC-MS 411.2 [M+H]⁺, 843.4 [2M+Na]⁺.

(ii) (3aR,6aS)-2-Ethoxy-4-methylphenyl 4-benzoyl-6-oxohexahydropyrrolo[3,2-b]pyrrole-1(2H)-carboxylate. Dess-Martin periodinane (56 mg, 0.13 mmol) was added to a solution of (3aR,6S,6aS)-2-ethoxy-4-methylphenyl 4-benzoyl-6-hydroxyhexahydropyrrolo[3,2-b]pyrrole-1(2H)-carboxylate (27 mg, 0.06 mmol) in dichloromethane (1 mL) under an atmosphere of argon. The mixture was stirred for 4 hours then Dess-Martin periodinane (56 mg, 0.13 mmol) added. The reaction was stirred for a further 18 hours then heated to 40° C. for 20 hours. The mixture was diluted with dichloromethane (10 mL) then the organic phase was washed with a mixture of 0.5M sodium thiosulphate:saturated aqueous sodium hydrogen carbonate (1:1, 8 mL), then saturated aqueous sodium hydrogen carbonate (8 mL), then brine (8 mL), then water (8 mL), then dried (Na₂SO₄), filtered and reduced in vacuo. Flash chromatography over silica, eluting with ethyl acetate:heptane mixtures 15:85 to 60:40 gave (3aR,6aS)-2-ethoxy-4-methylphenyl 4-benzoyl-6-oxohexahydropyrrolo[3,2-b]pyrrole-1 (2H)-carboxylate as a white solid (17 mg, 69%). TLC (R_{f}=0.21, EtOAc:heptane 3:1), analytical TALC single main peak, Rₜ=15.04 min., HPLC-MS 409.2 [M+H]⁺, 427.2 [M+H₂O+H]⁺, 839.4 [2M+Na]⁺, 875.4 [2(M+H₂O)+Na]⁺.

### Example 55

### (3aR,6aS)-2-Isopropoxyphenyl 4-benzoyl-6-oxohexahydropyrrolo[3,2-b]pyrrole-1(2H)-carboxylate

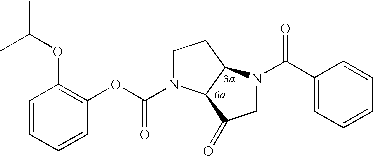

(i) (3aR,6S,6aS)-2-Isopropoxyphenyl 4-benzoyl-6-hydroxyhexahydropyrrolo[3,2-b]pyrrole-1(2H)-carboxylate. 1,1′,-Carbonyldiimidazole (42 mg, 0.26 mmol) in dichloromethane (0.5 mL) was added to a stirred solution of 2-isopropoxyphenol (33 mg, 0.22 mmol) in dichloromethane (0.5 mL). The mixture was heated at 40° C. for 2 hours then diluted with dichloromethane (10 mL). The solution was washed with water (7 mL) then dried (Na₂SO₄), filtered and reduced in vacuo to give 2-isopropoxyphenyl 1H-imidazole-1-carboxylate which was used without further purification

A solution of 2-isopropoxyphenyl 1H-imidazole-1-carboxylate (assumed to be 0.22 mmol, prepared as above) in dichloromethane (0.5 mL) was added to ((3S,3aS,6aR)-3-hydroxyhexahydropyrrolo[3,2-b]pyrrol-1(2H)-yl)(phenyl)methanone (60 mg, 0.26 mmol) in dichloromethane (0.5 mL). The mixture was heated at 40° C. with stirring for 2 days then concentrated in vacuo. The residue was purified by short path chromatography using an ISOLUTE SPE PE-AX/SCX-2 cartridge eluting with methanol:dichloromethane mixtures 0:100 to 10:90 followed by short path flash chromatography over silica, eluting with ethyl acetate:heptane mixtures 0:100 to 100:0 to give (3aR,6S,6aS)-2-isopropoxyphenyl 4-benzoyl-6-hydroxyhexahydropyrrolo[3,2-b]pyrrole-1(2H)-carboxylate as a colourless oil (28 mg). TLC (R_{f}=0.07, EtOAc:heptane 1:1), HPLC-MS 411.2 [M+H]⁺, 843.4 [2M+Na]⁺.

(ii) (3aR,6aS)-2-Isopropoxyphenyl 4-benzoyl-6-oxohexahydropyrrolo[3,2-b]pyrrole 1(2H)-carboxylate. Dess-Martin periodinane (116 mg, 0.27 mmol) was added to a solution of (3aR,6S,6aS)-2-isopropoxyphenyl 4-benzoyl-6-hydroxyhexahydropyrrolo[3,2-b]pyrrole-1(2H)-carboxylate (28 mg, 0.07 mmol) in dichloromethane (1 mL) under an atmosphere of argon. The mixture was heated at 40° C. with stirring for 24 hours. The mixture was diluted with dichloromethane (10 mL) then the organic phase was washed with a mixture of 0.5M sodium thiosulphate:saturated aqueous sodium hydrogen carbonate (1:1, 8 mL), then saturated aqueous sodium hydrogen carbonate (8 mL), then brine (8 mL), then water (8 mL), then dried Na₂SO₄), filtered and reduced in vacuo. Flash chromatography over silica, eluting with ethyl acetate:heptane mixtures 15:85 to 60:40 gave (3aR,6aS)-2-isopropoxyphenyl 4-benzoyl-6-oxohexahydropyrrolo[3,2-b]pyrrole-1(2H)-carboxylate as a white solid (18 mg, 63%). TLC (R_{f}=0.24, EtOAc:heptane 3:1), analytical HPLC single main peak, Rₜ=14.78 min., HPLC-MS 409.2 [M+H]⁺, 427.2 [M+H₂O+H]⁺, 839.3 [2M+Na]⁺, 875.4 [2(M+H₂O)+Na]⁺.

### Example 56

### (3aR,6aS)-2-Propylphenyl 4-benzoyl-6-oxohexahydropyrrolo[3,2-b]pyrrole-1(2H)-carboxylate

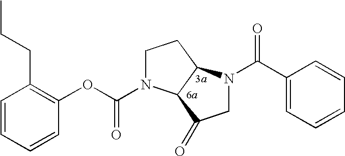

(i) (3aR,6S,6aS)-2-Propylphenyl 4-benzoyl-6-hydroxyhexahydropyrrolo[3,2-b]pyrrole-1(2H)-carboxylate. 1,1′-Carbonyldiimidazole (35 mg, 0.21 mmol) in dichloromethane (0.5 mL) was added to a stirred solution of 2-propylphenol (24 mg, 0.18 mmol) in dichloromethane (0.5 mL). The mixture was heated at 40° C. for 2 hours then diluted with dichloromethane (10 mL). The solution was washed with water (7 mL) then dried (Na₂SO₄), filtered and reduced in vacuo to give 2-propylphenyl 1H-imidazole-1-carboxylate which was used without further purification.

A solution of 2-propylphenyl 1H-imidazole-1-carboxylate (assumed to be 0.18 mmol, prepared as above) in tetrahydrofuran (1 mL) was added to ((3S,3aS,6aR)-3-hydroxyhexahydropyrrolo[3,2-b]pyrrol-1(2H)-yl)(phenyl)methanone (60 mg, 0.26 mmol). The mixture was heated at 65° C. with stirring for 4 days then concentrated in vacuo. The residue was purified by short path chromatography using an ISOLUTE SPE PE-AX/SCX-2 cartridge eluting with methanol:dichloromethane mixtures 0:100 to 10:90 followed by short path flash chromatography over silica, eluting with ethyl acetate:heptane mixtures 0:100 to 100:0 to give (3aR,6S,6aS)-2-propylphenyl 4-benzoyl-6-hydroxyhexahydropyrrolo[3,2-b]pyrrole-1(2H)-carboxylate as a colourless oil (26 mg). TLC (R_{f}=0.06, EtOAc:heptane 1:1), HPLC-MS 395.2 [M+H]⁺, 811.4 [2M+Na]⁺.

(ii) (3aR,6aS)-2-Propylphenyl 4-benzoyl-6-oxohexahydropyrrolo[3,2-b]pyrrole-1(2H)-carboxylate. Dess-Martin periodinane (56 mg, 0.13 mmol) was added to a solution of (3aR,6S,6aS)-2-propylphenyl 4-benzoyl-6-hydroxyhexahydropyrrolo[3,2-b]pyrrole-1(2H)-carboxylate (26 mg, 0.07 mmol) in dichloromethane (1 mL) under an atmosphere of argon. The mixture was stirred for 1.5 hours then Dess-Martin perodinane (56 mg, 0.13 mmol) was added and stirring continued for 18 hours. Dess-Martin periodinane (56 mg, 0.13 mmol) was added and the mixture stirred for 24 hours. The mixture was diluted with dichloromethane (10 mL) then the organic phase was washed with a mixture of 0.5M sodium thiosulphate:saturated aqueous sodium hydrogen carbonate (1:1, 8 mL), then saturated aqueous sodium hydrogen carbonate (8 mL), then brine (8 mL), then water (8 mL), then dried Na₂SO₄), filtered and reduced in vacuo. Flash chromatography over silica, eluting with ethyl acetate:heptane mixtures 15:85 to 60:40 gave (3aR,6aS)-2-propylphenyl 4-benzoyl-6-oxohexahydropyrrolo[3,2-b]pyrrole-1(2H)-carboxylate as a white solid (16 mg, 58%). TLC (R_{f}=0.46, EtOAc:heptane 3:1), analytical HPLC broad main peak, Rₜ=16.40-18.59 min., HPLC-MS 393.2 [M+H]⁺, 411.2 [M+H₂O+H]⁺, 807.3 [2M+Na]⁺, 843.4 [2(M+H₂O)+Na]⁺.

### Example 57

### (3aR,6aS)-(2-Methyl-6-(trifluoromethyl)pyridin-3-yl)methyl 4-benzoyl-6-oxohexahydropyrrolo[3,2-b]pyrrole-1(2H)-carboxylate

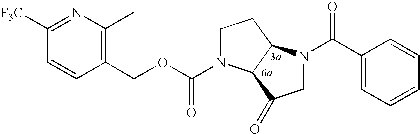

(i) (3aR,6S,6aS)-(2-Methyl-6-(trifluoromethyl)pyridin-3-yl)methyl 4-benzoyl-6-hydroxyhexahydropyrrolo[3,2-b]pyrrole-1(2H)-carboxylate. 1,1′-Carbonyldiimidazole (35 mg, 0.22 mmol) in dichloromethane (0.5 mL) was added to a stirred solution of (2-methyl-6-(trifluoromethyl)pyridin-3-yl)methanol (34 mg, 0.18 mmol) in dichloromethane (0.5 mL). The mixture was heated at 40° C. for 3 hours then diluted with dichloromethane (10 mL). The solution was washed with water (7 mL) then dried (Na₂SO₄), filtered and reduced in vacuo to give (2-methyl-6-(trifluoromethyl)pyridin-3-yl)methyl 1H-imidazole-1-carboxylate which was used without further purification.

A solution of (2-methyl-6-(trifluoromethyl)pyridin-3-yl)methyl 1H-imidazole-1-carboxylate (assumed to be 0.18 mmol, prepared as above) in dichloromethane (1 mL) was added to ((3S,3aS,6aR)-3-hydroxyhexahydropyrrolo[3,2-b]pyrrol-1(2H)-yl)(phenyl)methanone (50 mg, 0.22 mmol). The mixture was heated at 40° C. with stirring for 3 days then concentrated in vacuo. The residue was purified by short path flash chromatography over silica, eluting with ethyl acetate:heptane mixtures 0:100 to 100:0 to give (3aR,6S,6aS)-(2-methyl-6-(trifluoromethyl)pyridin-3-yl)methyl 4-benzoyl-6-hydroxyhexahydropyrrolo[3,2-b]pyrrole-1(2H)-carboxylate as a white solid (65 mg). TLC (R_{f}=0.15, EtOAc:heptane 4:1), analytical HPLC single main peak, Rₜ=14.68 min., HPLC-MS 450.2 [M+H]⁺, 472.2 [M+Na]⁺.

(ii) (3aR,6aS)-(2-Methyl-6-(trifluoromethyl)pyridin-3-yl)methyl 4-benzoyl-6-oxohexahydropyrrolo[3,2-b]pyrrole-1(2H)-carboxylate. Dess-Martin periodinane (234 mg, 0.55 mmol) was added to a solution of (3aR,6S,6aS)-(2-methyl-6-(trifluoromethyl)pyridin-3-yl)methyl 4-benzoyl-6-hydroxyhexahydropyrrolo[3,2-b]pyrrole-1(2H)-carboxylate (62 mg, 0.14 mmol) in dichloromethane (1 mL) under an atmosphere of argon. The mixture was stirred for 24 hours then diluted with dichloromethane (10 mL) then the organic phase was washed with a mixture of 0.5M sodium thiosulphate:saturated aqueous sodium hydrogen carbonate (1:1, 8 mL), then saturated aqueous sodium hydrogen carbonate (8 mL), then brine (8 mL), then water (8 mL), then dried (Na₂SO₄), filtered and reduced in vacuo. Flash chromatography over silica, eluting with ethyl acetate:heptane mixtures 15:85 to 65:35 gave (3aR,6aS)-(2-methyl-6-(trifluoromethyl)pyridin-3-yl)methyl 4-benzoyl-6-oxohexahydropyrrolo[3,2-b]pyrrole-1(2H)-carboxylate as a white solid (48 mg, 77%). TLC (R_{f}=0.29, EtOAc:heptane 4:1), analytical HPLC broad main peak, Rₜ=13.97-15.74 min., HPLC-MS 448.2 [M+H]⁺, 488.1 [M+H₂O+H]⁺.

### Example 58

### (3aR,6aS)-2-Fluoro-6-(trifluoromethyl)benzyl 4-benzoyl-6-oxohexahydropyrrolo[3,2-b]pyrrole-1(2H)-carboxylate

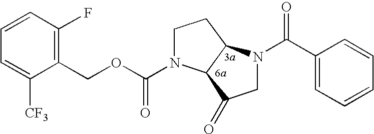

(i) (3aR,6S,6aS)-2-Fluoro-6-(trifluoromethyl)benzyl 4-benzoyl-6-hydroxyhexahydropyrrolo[3,2-b]pyrrole-1(2H)-carboxylate. 1,1′-Carbonyldiimidazole (52 mg, 0.32 mmol) in dichloromethane (1 mL) was added to (2-fluoro-6-(trifluoromethyl)phenyl)methanol (52 mg, 0.27 mmol). The mixture was heated at 40° C. for 2 hours with stirring. 1,1′-Carbonyldiimidazole (15 mg, 0.09 mmol) was added and stirring continued for 1 hour then diluted with dichloromethane (10 mL). The solution was washed with water (7 mL) then dried (Na₂SO₄), filtered and reduced in vacuo to give 2-fluoro-6-(trifluoromethyl)benzyl 1H-imidazole-1-carboxylate which was used without further purification.

A solution of (2-fluoro-6-(trifluoromethyl)benzyl 1H-imidazole-1-carboxylate (assumed to be 0.27 mmol, prepared as above) in dichloromethane (1 mL) was added to a solution of ((3S,3aS,6aR)-3-hydroxyhexahydropyrrolo[3,2-b]pyrrol-1(2H)-yl)(phenyl)methanone (50 mg, 0.22 mmol) in dichloromethane (1 mL). The mixture was heated at 40° C. with stirring for 4 days then concentrated in vacuo. The residue was purified by short path flash chromatography over silica, eluting with ethyl acetate:heptane mixtures 20:80 to 60:40 to give (3aR,6S,6aS)-2-fluoro-6-(trifluoromethyl)benzyl 4-benzoyl-6-hydroxyhexahydropyrrolo[3,2-b]pyrrole-1(2H)-carboxylate as a white solid (105 mg). TLC (R_{f}=0.16, EtOAc:heptane 1:1), HPLC-MS 453.1 [M+H]⁺, 927.2 [2M+Na]⁺.

(ii) (3aR,6aS)-2-Fluoro-6-(trifluoromethyl)benzyl 4-benzoyl-6-oxohexahydropyrrolo[3,2-b]pyrrole-1(2H)-carboxylate. Dess-Martin periodinane (150 mg, 0.35 mmol) was added to a solution of (3aR,6S,6aS)-2-fluoro-6-(trifluoromethyl)benzyl 4-benzoyl-6-hydroxyhexahydropyrrolo[3,2-b]pyrrole-1(2H)-carboxylate (80 mg, 0.18 mmol) in dichloromethane (4 mL) under an atmosphere of argon. The mixture was stirred for 2 hours then Dess-Martin periodinane (150 mg, 0.35 mmol) was added and stirred for 18 hours. The mixture was diluted with dichloromethane (20 mL) then the organic phase was washed with a mixture of 0.5M sodium thiosulphate:saturated aqueous sodium hydrogen carbonate (1:1, 15 mL), then saturated aqueous sodium hydrogen carbonate (15 mL), then brine (10 mL), then dried (Na₂SO₄), filtered and reduced in vacuo. Flash chromatography over silica, eluting with ethyl acetate:heptane mixtures 30:70 to 50:50 gave (3aR,6aS)-2-fluoro-6-(trifluoromethyl)benzyl 4-benzoyl-6-oxohexahydropyrrolo[3,2-b]pyrrole-1(2H)-carboxylate as a white solid (47 mg, 58%). TLC (R_{f}=0.59, EtOAc:heptane 4:1), analytical HPLC broad main peak, Rₜ=15.91-17.60 min., HPLC-MS 451.2 [M+H]⁺, 469.2 [M+H₂O+H]⁺, 923.2 [2M+Na]⁺, 959.2 [2(M+H₂O)+Na]⁺.

### Example 59

### (3aR,6aS)-6-Chloro-2-fluoro-3-methylbenzyl 4-benzoyl-6-oxohexahydropyrrolo[3,2-b]pyrrole-1(2H)-carboxylate

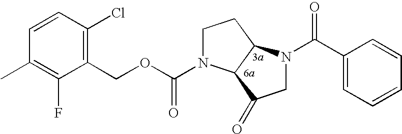

(i) (3aR,6S,6aS)-6-Chloro-2-fluoro-3-methylbenzyl 4-benzoyl-6-hydroxyhexahydropyrrolo[3,2-b]pyrrole-1(2H)-carboxylate. 1,1′-Carbonyldiimidazole (14 mg, 0.09 mmol) in dichloromethane (0.5 mL) was added to a stirred solution of (6-chloro-2-fluoro-3-methylphenyl)methanol (10 mg, 0.07 mmol) in dichloromethane (0.5 mL). The mixture was heated at 40° C. for 30 minutes then diluted with dichloromethane (10 mL). The solution was washed with water (7 mL) then dried (Na₂SO₄), filtered and reduced in vacuo to give 6-chloro-2-fluoro-3-methylbenzyl 1H-imidazole-1-carboxylate which was used without further purification.

A solution of 6-chloro-2-fluoro-3-methylbenzyl 1H-imidazole-1-carboxylate (assumed to be 0.07 mmol, prepared as above) in dichloromethane (0.5 mL) was added to ((3S,3aS,6aR)-3-hydroxyhexahydropyrrolo[3,2-b]pyrrol-1(2H)-yl)(phenyl)methanone (20 mg, 0.09 mmol). The mixture was heated at 40° C. with stirring for 6 days then concentrated in vacuo. The residue was purified by short path chromatography using an ISOLUTE SPE PE-AX/SCX-2 cartridge eluting with methanol:dichloromethane mixtures 0:100 to 10:90 to give (3aR,6S,6aS)-6-chloro-2-fluoro-3-methylbenzyl 4-benzoyl-6-hydroxyhexahydropyrrolo[3,2-b]pyrrole-1(2H)-carboxylate as a colourless oil (15 mg). TLC (R_{f}=0.04, EtOAc:heptane 1:1), HPLC-MS 433.2 [M+H]⁺, 887.3 [2M+Na]⁺.

(ii) (3aR,6aS)-6-Chloro-2-fluoro-3-methylbenzyl 4-benzoyl-6-oxohexahydropyrrolo[3,2-b]pyrrole-1(2H)-carboxylate. Dess-Martin periodinane (78 mg, 0.18 mmol) was added to a solution of (3aR,6S,64S)-6-chloro-2-fluoro-3-methylbenzyl 4-benzoyl-6-hydroxyhexahydropyrrolo[3,2-b]pyrrole-1(2H)-carboxylate (20 mg, 0.05 mmol) in dichloromethane (1 mL) under an atmosphere of argon. The mixture was stirred for 18 hours then heated at 40° C. for 4 hours. Dess-Martin periodinane (39 mg, 0.09 mmol) was added and the mixture heated at 40° C. with stirring for 3 hours then stirred at ambient temperature for 3 days. The mixture was diluted with dichloromethane (10 mL) then the organic phase was washed with a mixture of 0.5M sodium thiosulphate:saturated aqueous sodium hydrogen carbonate (1:1, 8 mL), then saturated aqueous sodium hydrogen carbonate (8 mL), then brine (8 mL), then water (8 mL), then dried (Na₂SO₄), filtered and reduced in vacuo. Flash chromatography over silica, eluting with ethyl acetate:heptane mixtures 15:85 to 60:40 gave (3aR,6aS)-6-chloro-2-fluoro-3-methylbenzyl 4-benzoyl-6-oxohexahydropyrrolo[3,2-b]pyrrole-1(2H)-carboxylate as a white solid (13 mg, 60%). TLC (R_{f}=0.40, EtOAc:heptane 3:1), analytical HPLC broad main peak, Rₜ=16.05-17.74 min., HPLC-MS 431.1 [M+H]⁺, 449.1 [M+H₂O+H]⁺, 883.2 [2M+Na]⁺, 919.2 [2(M+H₂O)+Na]⁺.

### Example 60

### (3aS,6aR)-2-Propylphenyl 3-oxotetrahydro-2H-furo[3,2-b]pyrrole-4(5H)-carboxylate

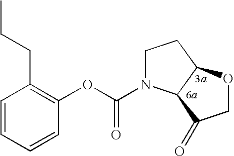

(i) (3R,3aR,6aR)-2-Propylphenyl 3-hydroxytetrahydro-2H-furo[3,2-b]pyrrole-4(5H)-carboxylate. 1,1′-Carbonyldiimidazole (51 mg, 0.32 mmol) in dichloromethane (1 mL) was added to 2-propylphenol (29 mg, 0.21 mmol). The mixture was heated at 40° C. for 2 hours then diluted with dichloromethane (10 mL). The solution was washed with water (7 mL) then dried (Na₂SO₄), filtered and reduced in vacuo to give 2-propylphenyl 1H-imidazole-1-carboxylate which was used without further purification.

A solution of 2-propylphenyl 1H-imidazole-1-carboxylate (assumed to be 0.21 mmol, prepared as above) in dichloromethane (1 mL) was added to (3R,3aR,6aR)hexahydro-2H-furo[3,2-b]pyrrol-3-ol hydrochloride (30 mg, 0.23 mmol). The mixture was heated at 40° C. with stirring for 3 days. Triethylamine (35 μL, 0.25 mmol) was added and the mixture heated at 40° C. with stirring for 1 day then concentrated in vacuo. The residue was dissolved in diethyl ether (20 mL) then washed with 1M hydrochloric acid (3×10 mL), then dried (MgSO₄), filtered and reduced in vacuo to give (3R,3aR,6aR)-2-propylphenyl 3-hydroxytetrahydro-2H-furo[3,2-b]pyrrole-4(5H)-carboxylate as a colourless oil (50 mg). HPLC-MS 292.2 [M+H]⁺.

(ii) (3aS,6aR)-2-Propylphenyl 3-oxotetrahydro-2H-furo[3,2-b]pyrrole-4(5H)-carboxylate. Dess-Martin periodinane (131 mg, 0.31 mmol) was added to a solution of (3R,3aR,6aR)-2-propylphenyl 3-hydroxytetrahydro-2H-furo[3,2-b]pyrrole-4(5H)-carboxylate (45 mg, 0.15 mmol) in dichloromethane (1 mL) under an atmosphere of argon. The mixture was stirred for 18 hours then diluted with dichloromethane (10 mL). The organic phase was washed with a mixture of 0.5M sodium thiosulphate:saturated aqueous sodium hydrogen carbonate (1:1, 6 mL), then saturated aqueous sodium hydrogen carbonate (6 mL), then brine (5 mL), then water (5 mL), then dried (Na₂SO₄), filtered and reduced in vacuo. Flash chromatography over silica, eluting with ethyl acetate:heptane mixtures 5:95 to 45:55 gave (3aS,6aR)-2-propylphenyl 3-oxotetrahydro-2H-furo[3,2-b]pyrrole-4(5H)-carboxylate as a pale yellow oil (5 mg, 12%). TLC (R_{f}=0.67, EtOAc:heptane 4:1), analytical HPLC broad main peak, Rₜ=11.64-14.06 min., HPLC-MS 290.1 [M+H]⁺, 308.1 [M+H₂O+H]⁺, 330.1 [M+H₂O+Na]⁺, 601.3 [2M+Na]⁺, 619.3 [2(M+H₂O)+Na]⁺.

### Assays for Cysteine Proteinase Activity

The compounds of the invention may be tested in one of a number of literature based biochemical assays that are designed to elucidate the characteristics of compound inhibition. The data from these types of assays enables compound potency and the rates of reaction to be measured and quantified. This information, either alone or in combination with other information, would allow the amount of compound required to produce a given pharmacological effect to be determined.

### General Materials and Methods

Unless otherwise stated, all general chemicals and biochemicals were purchased from either the Sigma Chemical Company, Poole, Dorset, U.K. or from Fisher Scientific UK, Loughborough, Leicestershire, U.K. Absorbance assays were carried out in flat-bottomed 96-well plates (Spectra; Greiner Bio-One Ltd., Stonehouse, Gloucestershire, U.K.) using a SpectraMax PLUS384 plate reader (Molecular Devices, Crawley, U.K.). Fluorescence high throughput assays were carried out in either 384-well microtitre plates (Corning Costar 3705 plates, Fisher Scientific) or 96-well ‘U’ bottomed Microfluor W1 microtitre plates (Thermo Labsystems, Ashford, Middlesex, U.K.). Fluorescence assays were monitored using a SpectraMax Gemini fluorescence plate reader (Molecular Devices). For substrates employing either a 7-amino-4-methylcoumarin (AMC) or a 7-amino-4-trifluoromethylcoumarin (AFC) fluorophore, assays were monitored at an excitation wavelength of 365 nm and an emission wavelength of 450 nm and the fluorescence plate reader calibrated with AMC. For substrates employing a 3-amino-benzoyl (Abz) fluorophore, assays were monitored at an excitation wavelength of 310 nm and an emission wavelength of 445 nm; the fluorescence plate reader calibrated with 3-amino-benzamide (Fluka). Unless otherwise indicated, all the peptidase substrates were purchased from Bachem UK, St. Helens, Merseyside, UK. Substrates utilizing fluorescence resonance energy transfer methodology (i.e. FRET-based substrates) were synthesized at Amura Therapeutics Limited using published methods (Atherton & Sheppard, Solid Phase Peptide Synthesis, IRL Press, Oxford, U.K., 1989) and employed Abz (2-aminobenzoyl) as the fluorescence donor and 3-nitro-tyrosine [Tyr(NO₂)] as the fluorescence quencher (Meldal, M. and Breddam, K., Anal. Biochem., 195, 141-147, 1991). Hydroxyethylpiperazine ethanesulfonate (HEPES), tris-hydroxylmethyl aminomethane (tris) base, bis-tris-propane and all the biological detergents (e.g. CHAPS, zwittergents, etc.) were purchased from CN Biosciences UK, Beeston, Nottinghamshire, U.K. Glycerol was purchased from Amersham Pharmacia Biotech, Little Chalfont, Buckinghamshire, U.K. Stock solutions of substrate or inhibitor were made up to 10 mM in 100% dimethylsulfoxide (DMSO) (Rathburns, Glasgow, U.K.) and diluted as appropriately required. In all cases the DMSO concentration in the assays was maintained at less than 1% (vol./vol.).

Assay protocols were based on literature precedent (Table 1; Barrett, A. J., Rawlings, N. D. and Woessner, J. F., 1998, Handbook of Proteolytic Enzymes, Academic Press, London and references therein) and modified as required to suit local assay protocols. Substrate was added as required to initiate the reaction and the activity, as judged by the change in fluorescence upon conversion of substrate to product, was monitored over time. All assays were carried out at 25±1° C. The results for selected compounds of the invention are shown in Table 1.

Trypanosoma cruzi Cruzipain Peptidase Activity Assays

Wild-type cruzipain, derived from Trypanosoma cruzi Dm28 epimastigotes, was obtained from Dr. Julio Scharfstein (Instituto de Biofisica Carlos Chagas Filho, Universidade Federal do Rio de Janeiro, Rio de Janeiro, Brazil). Activity assays were carried out in 100 mM sodium phosphate, pH 6.75 containing 1 mM EDTA and 10 mM L-cysteine using 2.5 nM enzyme. Ac-Phe-Arg-AMC (K_{M}^{app}≈12 μM) and D-Val-Leu-Lys-AMC (K_{M}^{app}≈4 μM) were used as the substrates. Routinely, Ac-FR-AMC was used at a concentration equivalent to K_{M}^{app} and D-Val-Leu-Lys-AMC was used at a concentration of 25 μM. The rate of conversion of substrate to product was derived from the slope of the increase in fluorescence monitored continuously over time.

Leishmania mexicana Cysteine Proteinase B (CPB) Peptidase Activity Assays

Wild-type recombinant CPB without the C-terminal extention (i.e. CPB2.8□CTE; Sanderson, S. J., et. al, Biochem. J, 347, 383-388, 2000) was obtained from Dr. Jeremy Mottram (Wellcome Centre for Molecular Parasitology, The Anderson College, University of Glasgow, Glasgow, U.K.). Activity assays were carried out in 100 mM sodium acetate; pH 5.5 containing 1 mM EDTA; 200 mM NaCl and 10 mM DTT (Alves, L. C., et. al., Mol. Biochem. Parasitol, 1,6,1-9, 2001) using 0.25 nM enzyme. Pro-Phe-Arg-AMC (K_{M}^{app}≈38 μM) was used as the substrate at a concentration equivalent to K_{M}^{app}. The rate of conversion of substrate to product was derived from the slope of the increase in fluorescence monitored continuously over time.

### Cathepsin Peptidase Activity Assays

Bovine cathepsin S, human cathepsin L, human cathepsin H and human cathepsin B were obtained from CN Biosciences. Recombinant human cathepsin S, human cathepsin K and human cathepsin X were obtained from Dr. Boris Turk (Josef Stefan Institute, Ljubljana, Slovenia). Unless otherwise stated, all peptidase activity assays were carried out in 10 mM bis-tris-propane (BTP), pH 6.5 containing 1 mM EDTA, 5 mM 2-mercaptoethanol and 1 mM CaCl₂. Human cathepsin H activity assays were carried out in 10 mM BTP pH 6.5, 142 mM NaCl₂ 1 mM CaCl₂, 1 mM EDTA, 1 mM DTT, 0.035 mM Zwittergent 3-16. Human cathepsin K assays were carried out in 100 mM sodium acetate; pH 5.5 containing 20 mM L-cysteine and 1 mM EDTA (Bossard, M. J., et. al., J. Biol. Chem., X, 12517-12524, 1996). Human cathepsin X assays were carried out in 100 mM sodium acetate; pH 5.5 containing 20 mM L-cysteine; 0.05% (w/v) Brij 35 and 1 mM EDTA (Santamaria, I., et. al., J. Biol. Chem., 273, 16816-16823, 1998; Klemencic, J, et al., Eur. J. Biochem., 267, 5404-5412, 2000). The final enzyme concentrations used in the assays were 0.5 nM bovine cathepsin S, 1 nM cathepsin L, 0.1 nM cathepsin B, 0.25 nM Cathepsin K; 1 nM cathepsin X and 10 nM cathepsin H. For the inhibition assays, the substrates used for cathepsin S, cathepsin L, cathepsin B, cathepsin K and cathepsin H were boc-Val-Leu-Lys-AMC (K_{M}^{app}≈30 μM), Ac-Phe-Arg-AMC (K_{M}^{app}≈2 μM), Z-Phe-Arg-AMC (K_{M}^{app}≈40 μM), Z-Leu-Arg-AMC (K_{M}^{app}≈2 μM); Bz-Phe-Val-Arg-AMC (K_{M}^{app}≈150 μM) respectively. In each case the substrate concentration used in each assay was equivalent to the K_{M}^{app}. The rate of conversion of substrate to product was derived from the slope of the increase in fluorescence monitored continuously over time.

### Trypsin Peptidase Activity Assays

Human pancreatic trypsin (iodination grade; CN Biosciences) activity assays were carried out in 10 mM HEPES, pH 8.0 containing 5 mM CaCl₂ using 0.1 nM trypsin. For the inhibition assays, Z-Gly-Gly-Arg-AMC (K_{M}^{app}≈84 μM) was used as the substrate at a concentration equivalent to K_{M}^{app}. The rate of conversion of substrate to product was derived from the slope of the increase in fluorescence monitored continuously over time.

### Clostripain Peptidase Activity Assays

Clostripain (Sigma) activity assays were carried out in 10 mM BTP, pH 6.5 containing 1 mM EDTA, 5 mM 2-mercaptoethanol and 1 mM CaCl₂ using 0.3 nM enzyme. For the inhibition assays, Z-Gly-Gly-Arg-AMC (K_{M}^{app}≈100 μM) was used as the substrate at a concentration equivalent to K_{M}^{app}. The rate of conversion of substrate to product was derived from the slope of the increase in fluorescence monitored continuously over time.

### Calpain Peptidase Activity Assays

Calpain (human erythrocyte μ-calpain and porcine kidney m-calpain; CN Biosciences) activity assays were carried out in 10 mM HEPES, pH 7.5 containing 2 mM 2-mercaptoethanol and CaCl₂ using 25 nM of either enzyme (Sasaki, et. al., J. Biol. Chem., 259, 12489-12494, 1984). For μ-calpain inhibition assays, the buffer contained 100 μM CaCl₂ and Abz-Ala-Asn-Leu-Gly-Arg-ProAla-Leu-Tyr(NO₂)-Asp-NH₂ (K_{M}^{app}≈20 μM; Amura Therapeutics Limited) was used as the substrate. For m-calpain inhibition assays, the assay buffer contained 200 μM CaCl₂ and Abz-Lys-Leu-Cys(Bzl)-Phe-Ser-Lys-Gln-Tyr(NO₂)-Asp-NH₂ (K_{M}^{app}≈22 μM; Amura Therapeutics Limited) was used as the substrate. In both cases the substrate concentration employed in the assays was equivalent to the K_{M}^{app}. The rate of conversion of substrate to product was derived from the slope of the increase in fluorescence monitored continuously over time.

Extracellular S. aureus V8 Cysteine Peptidase (Staphylopain) Peptidase Activity Assays

S. aureus V8 was obtained from Prof. S. Arvidson, Karolinska Institute, Stockholm, Sweden. Extracellular S. aureus V8 cysteine peptidase (staphylopain) activity assays were carried out using partially purified S. aureus V8 culture supernatant (obtained from Dr. Peter Lambert, Aston University, Birmingham, U.K.). Activity assays were carried out in 10 mM BTP, pH 6.5 containing 1 mM EDTA, 5 mM 2-mercaptoethanol and 1 mM CaCl₂ using two-times diluted partially purified extract. For the inhibition assays, Abz-Ile-Ala-Ala-Pro-Tyr(NO₂)-Glu-NH₂ (K_{M}^{app}≈117 μM; Amura Therapeutics Limited) was used as the substrate at a concentration equivalent to K_{M}^{app}. The rate of conversion of substrate to product was derived from the slope of the increase in fluorescence monitored continuously over time.

### Foot-and-Mouth Disease Leader Peptidase (FMDV-LP) Activity Assays

Recombinant wild-type FMDV-LP was obtained from Dr. Tim Skem (Institut für Medizinische Biochemie, Abteilung für Biochemie, Universtät Wien, Wien, Austria). Activity assays were carried out in 50 mM tris-acetate, pH 8.4 containing 1 mM EDTA, 10 mM L-cysteine and 0.25% (w/v) CHAPS using 10 nM enzyme. For the inhibition assays, Abz-Arg-Lys-Leu-Lys-Gly-Ala-Gly-Ser-Tyr602)-Glu-NH₂ (K_{M}^{app}≈51 μM, Amura Therapeutics Limited) was used as the substrate at a concentration equivalent to K_{M}^{app}. The rate of conversion of substrate to product was derived from the slope of the increase in fluorescence monitored continuously over time.

### Caspase Peptidase Activity Assays

Caspases 1-10 were obtained from CN Biosciences or BioVision Inc. (Mountain View, Calif., USA) and all assays were carried out in 50 mM HEPES; pH 7.2, 10% (v/v) glycerol, 0.1% (w/v) CHAPS, 142 mM NaCl, 1 mM EDTA, 5 mM dithiothreitol (DTT) using 0.1-1 Upper assay. For caspase 1, Ac-Leu-Glu-His-Asp-AMC was used as the substrate; for caspase 2, Z-Val-Asp-Val-Ala-Asp-AFC was used as the substrate; for caspase 3, Ac-Asp-Glu-Val-Asp-AMC was used as the substrate; for caspase 4, Suc-Tyr-Val-Ala-Asp-AMC was used as the substrate; for caspase 5, Ac-Leu-Glu-His-Asp-AMC was used as the substrate; for caspase 6, Ac-Val-Glu-Ile-Asp-AMC was used as the substrate; for caspase 7, Ac-Asp-Glu-Val-Asp-AMC was used as the substrate; for caspase 8, Ac-Ile-Glu-Thr-AspAMC was used as the substrate; for caspase 9, Ac-Leu-Glu-His-Asp-AMC was used as the substrate; for caspase 10, Ac-Ile-Glu-Thr-Asp-AMC was used as the substrate (Nicholson, D. W. and Thornberry, N.A., TIBS, 22a299-306, 1997; Stennicke, H. R. and Salvesen, G. S., J. Biol. Chem., 272(41), 25719-25723, 1997; Talanian, R. V., et. al., J. Biol. Chem., 272(15), 9677-9682, 1997; Wolf, B. B. and Green, D. R., J. Biol. Chem., 274(29), 20049-20052, 1999). The rate of conversion of substrate to product was derived from the slope of the increase in fluorescence monitored continuously over time.

### Measurement of the Apparent Macroscopic Binding (Michaelis) Constants (K_{M}^{app}) for Substrates

The apparent macroscopic binding constant (K_{M}^{app}) for each substrate was calculated, from the dependence of enzyme activity as a function of substrate concentration. The observed rates were plotted on the ordinate against the related substrate concentration on the abscissa and the data fitted by direct regression analysis (Prism v 3.02; GraphPad, San Diego, USA) using Equation 1 (Cornish-Bowden, A. Fundamentals of enzyme kinetics Portland Press; 1995, 93-128).

$${v}_{i} = \frac{{V}_{max}^{app} \cdot [ {S}_{o} ]}{[ {S}_{o} ] + {K}_{M}^{app}} \qquad ( 1 )$$

In Equation 1 ‘vᵢ’ is the observed initial rate, ‘Vₘₐₓ^{app}’ is the observed maximum activity at saturating substrate concentration, ‘K_{M}^{app}’ is the apparent macroscopic binding (Michaelis) constant for the substrate, ‘[Sₒ]’ is the initial substrate concentration.

### Measurement of the Inhibition Constants

The apparent inhibition constant (Kᵢ) for each compound was determined on the basis that inhibition was reversible and occurred by a pure-competitive mechanism. The Kᵢ values were calculated, from the dependence of enzyme activity as a function of inhibitor concentration, by direct regression analysis (Prism v 3.02) using Equation 2 (Cornish-Bowden, A., 1995).

$${v}_{i} = \frac{{V}_{max}^{app} \cdot [ S ]}{[ S ] + \{ {K}_{M}^{app} \cdot ( [ I ] / {K}_{i} ) \}} \qquad ( 2 )$$

In Equation 2 ‘vᵢ’ is the observed residual activity, ‘Vₘₐₓ^{app}’ is the observed maximum activity (i.e. in the absence of inhibitor), ‘K_{M}^{app}’ is the apparent macroscopic binding (Michaelis) constant for the substrate, ‘[S]’ is the initial substrate concentration, ‘Kᵢ’ is the apparent dissociation constant and ‘[I]’ is the inhibitor concentration.

In situations where the apparent dissociation constant (Kᵢ^{app}) approached the enzyme concentrations, the Kᵢ^{app} values were calculated using a quadratic solution in the form described by Equation 3 (Morrison, J. F. Trends Biochem. Sci., 102-105, 1982; Morrison, J. F. Biochim. Biophys. Acta. a, 269-286, 1969; Stone, S. R and Hofsteenge, J. Biochemistry, 25, 4622-4628, 1986).

$$\begin{matrix}{v}_{i} = \frac{F \{ \begin{matrix}{E}_{o} - {I}_{o} - {K}_{i}^{app} + \\ \sqrt{{( {E}_{o} - {I}_{o} - {K}_{i}^{app} )}^{2} + 4 \cdot {K}_{i}^{app} \cdot {E}_{o}}\end{matrix} \}}{2} & ( 3 ) \\ {K}_{i}^{app} = {K}_{i} ( 1 + [ {S}_{o} ] / {K}_{M}^{app} ) & ( 4 )\end{matrix}$$

In Equation 3 ‘vᵢ’ is the observed residual activity, ‘F’ is the difference between the maximum activity (i.e. in the absence of inhibitor) and minimum enzyme activity, ‘Eₒ’ is the total enzyme concentration, ‘Kᵢ^{app}’ is the apparent dissociation constant and ‘Iₒ’ is the inhibitor concentration. Curves were fitted by non-linear regression analysis (Prism) using a fixed value for the enzyme concentration. Equation 4 was used to account for the substrate kinetics, where ‘Kᵢ’ is the inhibition constant, ‘[Sₒ]’ is the initial substrate concentration and ‘K_{M}^{app}’ is the apparent macroscopic binding (Michaelis) constant for the substrate (Morrison, 1982).

The Second-Order Rate of Reaction of Inhibitor with Enzyme

Where applicable, the concentration dependence of the observed rate of reaction (k_{obs}) of each compound with enzyme was analysed by determining the rate of enzyme inactivation under pseudo-first order conditions in the presence of substrate (Morrison, J. F., TIBS, 102-105, 1982; Tian, W. X. and Tsou, C. L., Biochemistry, 21, 1028-1032, 1982; Morrison, J. F. and Walsh, C. T., from Meister (Ed.), Advances in Enzymol., 61, 201-301, 1988; Tsou, C. L., from Meister (Ed.), Advances in Enzymol., X, 381436, 1988). Assays were carried out by addition of various concentrations of inhibitor to assay buffer containing substrate. Assays were initiated by the addition of enzyme to the reaction mixture and the change in fluorescence monitored over time. During the course of the assay less than 10% of the substrate was consumed.

$$F = {v}_{s} t + \frac{( {v}_{o} - {v}_{s} ) ⌊ 1 - {e}^{( {k}_{obs} \cdot i )} ⌋}{{k}_{obs}} + D \qquad ( 5 )$$

The activity fluorescence progress curves were fitted by non-linear regression analysis (Prism) using Eq. 5 (Morrison, 1969; Morrison, 1982); where ‘F’ is the fluorescence response, ‘t’ is time, ‘vₒ’ is the initial velocity, ‘v_{g}’ is the equilibrium steady-state velocity, ‘k_{obs}’ is the observed pseudo first-order rate constant and ‘D’ is the intercept at time zero (i.e. the ordinate displacement of the curve). The second order rate constant was obtained from the slope of the line of a plot of k_{obs} versus the inhibitor concentration (i.e. k_{obs}/[I]). To correct for substrate kinetics, Eq. 6 was used, where ‘[Sₒ]’ is the initial substrate concentration and ‘K_{M}^{app}’ is the apparent macroscopic binding (Michaelis) constant for the substrate.

$${k}_{inact} = \frac{{k}_{obs} ( 1 + [ {S}_{o} ] / {K}_{M}^{app} )}{[ I ]} \qquad ( 6 )$$

Compounds of the invention were tested by the above described assays and observed to exhibit cathepsin K inhibitory activity or inhibitory activity against an alternative CAC1 proteinase with an in vitro Kᵢ<100 μM.

Various modifications and variations of the described aspects of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the relevant fields are intended to be within the scope of the following claims.

### REFERENCES

^{a} Barrett, A. J., Biochem. J, 1, 909-912, 1980
^{b} Barrett, A. J. and Kirschke, H., Methods Enzymol, 80, 535-561, 1981
^{c} Quibell, M. and Taylor, S., WO0069855, 2000
^{d} Bromme, D., Steinert, Freibe, S., Fittkau, S., Wiederanders, B., and Kirschke, H., Biochem. J, 2, 475-481, 1989
^{e} Rano, T. A., et. al., Chem. Biol, 4, 149, 1997
^{f} Talanian, R. V., et. al., J. Biol. Chem., 272, 9677, 1997
^{g} Lazebnik, Y. A., Kaufmann, S. H., Desnoyers, S., Poirer, G. G. and Earnshaw, W. C., Nature., 371, 768-774, 1994
^{h} Han, Z., et. al., J. Biol. Chem., 13432, 1997
ⁱ Takahashi, A., et. al., PNAS, 93, 8395, 1996
^{j} Martins, L. M., et. al., J. Biol. Chem., 272, 7421, 1997
^{k} Nagata, S., Cell., 88, 355, 1997
^{l} Harris, J. L., et al., J. Biol. Chem., 273, 27364, 1998
^{m} Cazzulo, J. J., Cazzulo Franke, M. C., Martinez, J. and Franke de Cazzulo, B. M., Biochim. Biophys. Acta., 1037, 186-191, 1990
ⁿ Cazzulo, J. J., Bravo, M., Raimondi, A., Engstrom, U., Lindeberg, G. and Hellman, U., Cell Mol. Biol, 42, 691-696, 1996
^{o} Potempa, J., Dubin, A., Korzus, G. and Travis, J., Biochem. J, 263, 2664-2667, 1988
^{p} Kembhavi, A. A., Buttle, D. J., Rauber, P. and Barrett, A. J., FEBS Lett., 283, 277-280,
^{q} Alves, L. C., et. al., Mol. Biochem. Parasitol, 116, 1-9, 2001.
^{r} Guarné, et al., J. Mol. Biol., 302, 1227-1240, 2000.
^{s} Halfon and Craik, (Barret, Rawlings and Woessner, eds.), in Handbook of Proteolytic Enymes, Academic Press, London, 12-21, 1998.
^{t} Sasaki, et. al., (1984), J. Biol. Chem., 259, 12489-12494, 1984.
^{u} Bossard, M. J., et. al., J. Biol. Chem., 21, 12517-12524, 1996
^{v} Santamaria, I., et. al., J. Biol. Chem., 273, 16816-16823, 1998
^{w} Klemencic, J, et al., Eur. J. Biochem., 267, 5404-5412, 2000

TABLE 1

The enzyme assays described herein were carried out
according to literature precedents.

| | | | |
|---|---|---|---|
| Enzyme | Buffer | Substrate | Reference |

| | | | |
|---|---|---|---|
| Cathepsin B | I | Z-Phe-Arg-AMC | a, b |
| Cathepsin H | II | Bz-Phe-Val-Arg-AMC | a, b |
| Cathepsin L | I | Ae-Phe-Arg-AMC | b, c |
| Cathepsin S | I | Boc-Val-Leu-Lys-AMC | c, d |
| Caspase 1 | III | Ac-Leu-Glu-His-Asp-AMC | e |
| Caspase 2 | III | Z-Val-Asp-Val-Ala-Asp-AFC | f |
| Caspase 3 | III | Ac-Asp-Glu-Val-Asp-AMC | g, h |
| Caspase 4 | III | Suc-Tyr-Val-Ala-Asp-AMC | f |
| Caspase 5 | III | Ac-Leu-Glu-His-Asp-AMC | |
| Caspase 6 | III | Ac-Val-Glu-Ile-Asp-AMC | i, j, k |
| Caspase 7 | III | Ac-Asp-Glu-Val-Asp-AMC | |
| Caspase 8 | III | Ac-Ile-Glu-Thr-Asp-AMC | l |
| Caspase 9 | III | Ac-Leu-Glu-His-Asp-AMC | |
| Caspase 10 | III | Ac-IIe-Glu-Thr-Asp-AMC | |
| Cruzipain | IV | D-Val-Leu-Lys-AMC | m, n |
| CPB2.8ΔCTE | XI | Pro-Phe-Arg-AMC | q |
| S. Aureus | I | Abz-Ile-Ala-Ala-Pro- | o |
| Extracellular | | Tyr(NO₂)-Glu-NH₂ | |
| cysteine | | | |
| peptidase | | | |
| Clostripain | | Z-Gly-Gly-Arg-AMC | p |
| FMDV LP | V | Abz-Arg-Lys-Leu-Lys-Gly- | r |
| | | Ala-Gly-Ser-Tyr(NO₂)-Glu- | |
| | | NH₂ | |
| Trypsin | VI | Z-Gly-Gly-Arg-AMC | s |
| Calpain μ | VII | Abz-Ala-Asn-Leu-Gly-Arg- | t |
| | | Pro-Ala-Leu-Tyr(NO₂)-Asp- | |
| | | NH₂ | |
| Calpain m | VIII | Abz-Lys-Leu-Cys(Bzl)-Phe- | t |
| | | Ser-Lys-Gln-Tyr(NO₂)-Asp- | |
| | | NH₂ | |
| Cathepsin K | IX | Z-Phe-Arg-AMC | u |
| Cathepsin X | X | | v, w |

I: 10 mM BTP, pH 6.5 containing 1 mM EDTA, 5 mM 2-mercaptoethanol and 1 mM CaCl₂
II: 10 mM BTP, pH 6.5 containing 1 mM EDTA, 142 mM NaCl, 1 mM DTT, 1 mM CaCl₂, 0.035 mM Zwittergent 3-16
III: 50 mM HEPES pH 7.2, 10% Glycerol, 0.1% CHAPS, 142 mM NaCl, 1 mM EDTA, 5 mM DTT
IV: 100 mM sodium phosphate, pH 6.75 containing 1 mM EDTA and 10 mM L-cysteine
V: 50 mM tris·acetate, pH 8.4 containing 1 mM EDTA, 10 mM L-cysteine and 0.25% (w/v) CHAPS
VI: 10 mM HEPES, pH 8.0 containing 5 mM CaCl₂
VII: 10 mM HEPES, pH 7.5 containing 2 mM 2-mercaptoethanol and 100 μM CaCl₂
VIII: 10 mM HEPES, pH 7.5 containing 2 mM 2-mercaptoethanol and 200 μM CaCl₂
IX: 100 mM sodium acetate; pH 5.5 containing 10 mM L-cysteine and 1 mM EDTA
X: 100 mM sodium acetate; pH 5.5 containing 10 mM L-cysteine; 0.05% (w/v) Brij 35 and 1 mM EDTA
XI: 100 mM sodium acetate; pH 5.5 containing 10 mM L-cysteine; 142 mM sodium chloride and 1 mM EDTA

1. A compound of formula (I), or a pharmaceutically acceptable salt thereof, 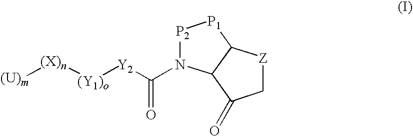
wherein:
Z is O, 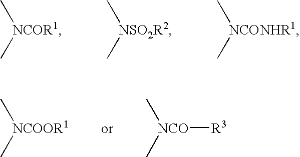
where R¹ and R² are each independently a hydrocarbyl group, and R³ is a saturated heterocycle defined by 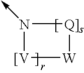
where
Q and V are each independently selected from 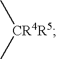
W is selected from 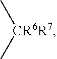
O, S, 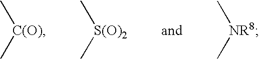
‘r’ and ‘s’ are each independently 1 or 2;
P₁ is 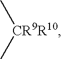
where R⁹ and R¹⁰ are each independently selected from H, alkyl, cycloalkyl, Ar-alkyl, Ar, halogen, alkoxy, hydroxyl and NR⁴⁶R⁴⁷, wherein R⁴⁶ and R⁴⁷ are each independently H or alkyl;
P₂ is O, 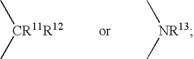
Y₂ is O, S or 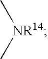
or where (U)ₘ, (X)ₙ and (Y₁)ₒ are absent, Y₂ is OR⁴⁸, SR⁴⁸ or —NR¹⁴R⁴⁴, where R⁴⁸ is alkyl, and R¹⁴ and R⁴⁴ are each independently selected from H and alkyl, or R¹⁴ and R⁴⁴ are linked to form a cyclic group together with the nitrogen to which they are attached;
each Y₁ is independently 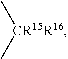
and ‘o’ is 0, 1, 2 or 3;
or when ‘o’ is 1, Y₁ may additionally be selected from 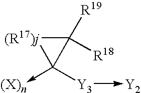
where Y₃ is methylene or absent;
R¹⁷ is selected from 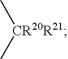
‘j’ is 1, 2, 3 or 4, where when ‘j’ is 2, 3 or 4, R¹⁷ may additionally be selected from O, S, SO₂, NR²² and —N(R²²)C(O);
or when ‘o’ is 1, 2, or 3 and (U)ₘ and (X)ₙ are absent, the terminal Y₁ group is selected from CR¹⁵R¹⁶R⁴² and 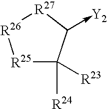
R²⁵ is selected from 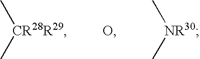
R²⁶ is selected from 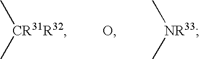
except when R²⁵ is O, then R²⁶ is selected from 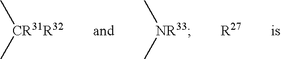
selected from 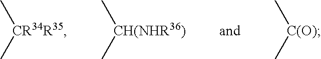
each X is independently 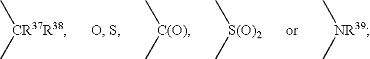
‘n’ is 0, 1 or 2, provided that when (Y₁)ₒ is absent, (X)ₙ is CR³⁷R³⁸ or is absent, and also provided that when ‘n’ is 2, (X)ₙ contains a minimum of one 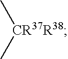
and when (U)ₘ is absent and n is 1 or 2, the terminal X group is CR³⁷R³⁸R⁴³;
each U is independently a 5- to 7-membered monocyclic or a 8- to 11-membered bicyclic ring which is either saturated or unsaturated and which includes up to four heteroatoms as shown below. 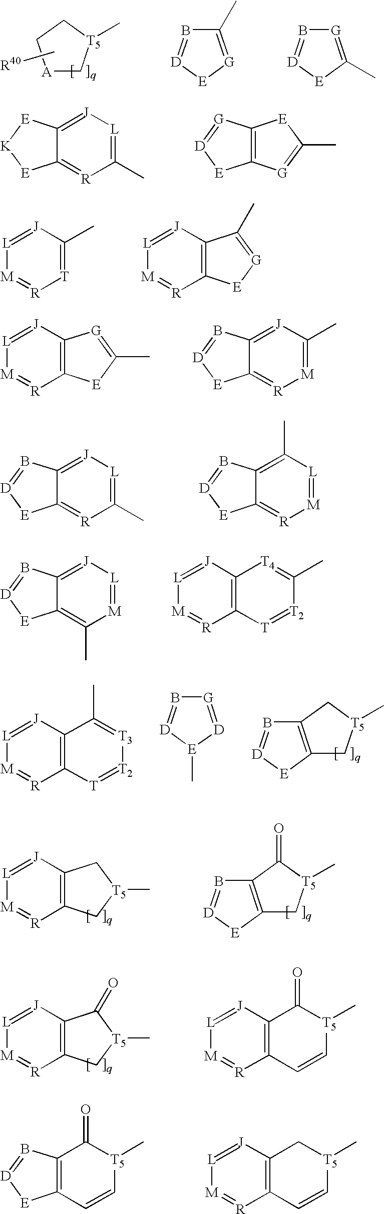 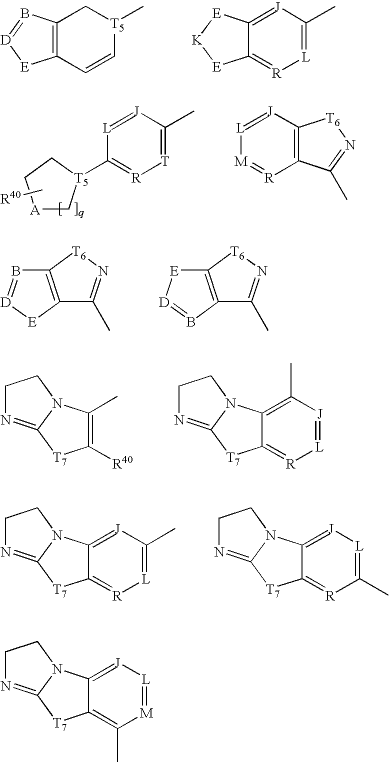
wherein R⁴⁰ is:
H, haloalkyl, alkyl, cycloalkyl, Ar-alkyl, Ar, OH, O-alkyl, O-cycloalkyl, O-alkyl, OAr, S-alkyl, SH, S-cycloalkyl, S—Ar-alkyl, SAr, SO₂-alkyl, NHCO-alkyl, SO₂H, SO₂-cycloalkyl, SO₂—Ar-alkyl, SO₂Ar, NH-alkyl, NH₂, NH-cycloalkyl, NH—Ar-alkyl, NHAr, N(alkyl)₂, NH₂, NH(alkyl), N(cycloalkyl)₂ or N(Ar-alkyl)₂ or NAr₂; or, when part of a CHR⁴⁰ or CR⁴⁰ group, R⁴⁰ may be halogen;
A is selected from:
CH 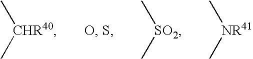
and N-oxide 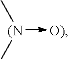
where R⁴⁰ is as defined above; and R⁴¹ is selected from H, alkyl, cycloalkyl, Ar and Ar-alkyl;
B, D and G are each independently selected from: 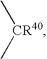
where R⁴⁰ is as defined above, N and N-oxide 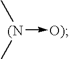
E is selected from:
CH₂, 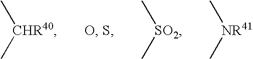
and N-oxide 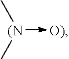
where
R⁴⁰ and R⁴¹ are defined as above;
K is selected from:
CH₂, 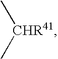
where R⁴¹ is defined as above;
J, L, M, R, T, T₂, T₃ and T₄ are independently selected from:
CR⁴⁰ where R⁴⁰ is as defined above, N and N-oxide 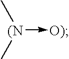
T₅ is selected from:
CH and N;
T₆ is selected from: 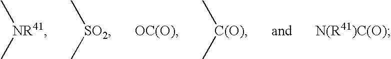
T₇ is selected from:
O, S, 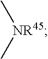
‘q’ is 1, 2 or 3;
‘m’ is 0 or 1;
R⁴⁻⁷, R¹¹⁻¹², R¹⁵⁻¹⁶, R¹⁸⁻²¹, R²³⁻²⁴, R²⁸⁻²⁹, R³¹⁻³², R³⁴⁻³⁵, R³⁷⁻³⁸ and R⁴²⁻⁴³ are each independently selected from H, alkyl, cycloalkyl, Ar-alkyl, Ar and halogen; and R⁸, R¹³, R²², R³⁰, R³³, R³⁶, R³⁹ and R⁴⁵ are each independently selected from H, alkyl, cycloalkyl, Ar-alkyl and Ar.

2. A compound according to claim 1 wherein R¹ and R² are each independently selected from alkyl, cycloalkyl, Ar-alkyl and Ar, each of which may be optionally substituted by one or more R⁴⁰, NO₂, CN, CF₃ and/or halo groups.

3. A compound according to claim 1 wherein R¹ is selected from alkyl and aryl, each of which may be optionally substituted by one or more R⁴⁰, NO₂, CN, CF₃ and/or halo groups.

4. A compound according to a claim 1 wherein P₁ is 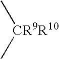
R⁹ and R¹⁰ are each independently H, alkyl, alkoxy, NR⁴⁶R⁴⁷ or halogen.

5. A compound according to claim 1 wherein P₁ is CH-halogen, CH₂, CH(OMe), CH(NH₂) or CH(NHMe).

6. A compound according to claim 1 wherein P₁ is CH₂.

7. A compound according to claim 1 wherein P₂ is 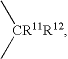
or NR¹³, and R¹¹⁻¹³ are each independently H or alkyl.

8. A compound according to claim 1 wherein P₂ is CH₂, O or NH.

9. A compound according to claim 1 wherein P₂ is CH₂.

10. A compound according to claim 1 wherein Z is O or NCOR¹.

11. A compound according to claim 1 wherein Z is O or NCOAr.

12. A compound according to claim 1 wherein Z is O or NCOPh.

13. A compound according to claim 1 wherein Y₂ is O, NH or S.

14. A compound according to claim 1 wherein Y₁ is

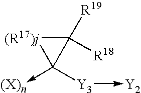

15. A compound according to claim 14 wherein R¹⁷ is CH₂, j is 2 and R¹⁹ and R¹⁸ are both H.

16. A compound according to claim 14 wherein Y₃ is absent.

17. A compound according to claim 14 wherein (Y₁)ₒ is cyclobutyl and o is 1.

18. A compound according to a claim 14 wherein (X)ₙ is CH₂O.

19. A compound according to claim 14 wherein U is 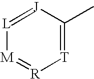
and J, L, M, R and T are each independently selected from CR⁴⁰.

20. A compound according to claim 14 wherein U is phenyl and m is 1.

21. A compound according to claim 1 wherein P₂ is 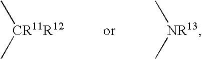
and the stereochemistry is (3aS,6aR) or (3aR,6aS).

22. A compound according to claim 1 wherein P₂ is O, and the stereochemistry is (3aS,6aS) or (3aR,6aR).

23. A compound according to claim 1 wherein P₂ is 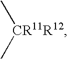
Z is O and the stereochemistry is (3aS,6aR).

24. A compound according to claim 1 wherein P₂ is O, Z is O, and the stereochemistry is (3aS,6aS).

25. A compound according to claim 1 wherein P₂ is 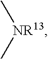
Z is O, and the stereochemistry is (3aR,6aS).

26. A compound according to claim 1 wherein P₂ is 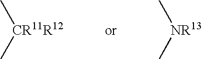
and Z is 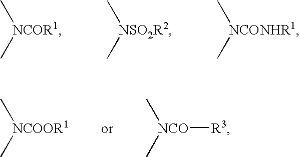
and the stereochemistry is (3aR,6aS).

27. A compound according to claim 1 wherein P₂ is O, Z is 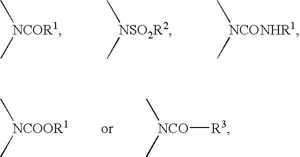
and the stereochemistry is (3aS,6aS).

28. A compound according to claim 1 wherein m is 0, (X)ₙ is CR³⁷R³⁸R⁴³ and n is 1.

29. A compound according to claim 28 wherein n is 1 and X is CH₃, CH(alkyl)₂ or C(alkyl)₃.

30. A compound according to claim 29 wherein n is 1 and X is CH₃, CH₂Me, CH(Me)₂ or CMe₃.

31. A compound according to claim 1 wherein o is 1 or 2 and each Y₁ is independently

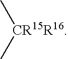

32. A compound according to claim 31 wherein R¹⁵ and R¹⁶ are each independently H or alkyl.

33. A compound according to claim 32 wherein (Y₁)ₒ is CHⁱPr, CHMe, CH₂, or CH(Me)CH₂.

34. A compound according to claim 1 wherein when ‘o’ is 1, 2, or 3 and (U)ₘ and (X)ₙ are absent, the terminal Y₁ group is

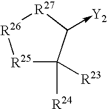

35. A compound according to claim 34 wherein R²⁷ is CO, R²⁶ is O, R²⁵ is CH₂ and R²³ and R²⁴ are both CH₃.

36. A compound according to claim 34 or claim 35 wherein o is 1.

37. A compound according to claim 1 wherein (U)ₘ, (X)ₙ and (Y₁)ₒ are absent, and Y₂ is —NR¹⁴R⁴⁴, where R¹⁴ and R⁴⁴ are linked to form a cyclic group together with the nitrogen to which they are attached.

38. A compound according to claim 37 wherein R¹⁴ and R⁴⁴ are linked to together with the nitrogen to which they are attached to form a pyrrolidine group.

39. A compound according to claim 1 wherein R¹ is alkyl optionally substituted by one or more NHCO-alkyl groups.

40. A compound according to claim 39 wherein R¹ is

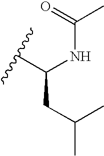

41. A compound according to claim 39 which is selected from the following:
(3aS,6aR)-3-Oxo-hexahydro-furo[3,2-b]pyrrole-4-carboxylic acid 1-phenoxymethyl-cyclobutyl ester [1];
(3aS,6aR)-3-Oxo-hexahydro-furo[3,2-b]pyrrole-4-carboxylic acid (1-phenoxymethyl-cyclobutyl)-amide [2];
(3aS,6aR)-3-Oxo-hexahydro-furo[3,2-b]pyrrole-4-carbothioic acid S-(1-phenoxy methyl-cyclobutyl)ester [3];
(3aS,6aS)-6-Oxo-tetrahydro-furo[3,2-c]isoxazole-1-carboxylic acid 1-phenoxymethyl-cyclobutyl ester [4];
(3aR,6aS)-6-Oxo-hexahydro-furo[3,2-c]pyrazole-1-carboxylic acid 1-phenoxymethyl-cyclobutyl ester [5];
(3aR,6aS)-4-Benzoyl-6-oxo-hexahydro-pyrrolo[3,2-b]pyrrole-1-carboxylic acid 1-phenoxymethyl-cyclobutyl ester [6];
(3aS,6aS)-4-Benzoyl-6-oxo-hexahydro-2-oxa-1,4-diaza-pentalene-1-carboxylic acid 1-phenoxymethyl-cyclobutyl ester [7];
(3aR,6aS)-4-Benzoyl-6-oxo-hexahydro-pyrrolo[3,2-c]pyrazole-1-carboxylic acid 1-phenoxymethyl-cyclobutyl ester [8];
(3aR,6aS)-4-Benzoyl-6-oxo-hexahydro-pyrrolo[3,2-b]pyrrole-1-carboxylic acid 1-isopropyl-2-methyl-propyl ester [9];
(3aR,6aS)-4-(2S-Acetylamino-4-methyl-pentanoyl)-6-oxo-hexahydro-pyrrolo[3,2-b]pyrrole-1-carboxylic acid 1-isopropyl-2-methyl-propyl ester [10];
(3aR,6aS)-(2S-Acetylamino-methyl-pentanoyl)-6-oxo-hexahydro-pyrrolo[3,2-b]pyrrole-1-carboxylic acid isobutyl ester [11];
(3aR,6aS)-4-(2S-Acetylamino-4-methyl-pentanoyl)-6-oxo-hexahydro-pyrrolo[3,2-b]pyrrole-1-carboxylic acid isopropyl ester [12];
(3aR,6aS)-4-(2S-Acetylamino-4-methyl-pentanoyl)-oxo-hexahydro-pyrrolo[3,2-b]pyrrole-1-carboxylic acid 2,2-dimethyl-propyl ester [13];
(3aR,6aS)-4-(2S-Acetylaminomethyl-pentanoyl)-6-oxo-hexahydro-pyrrolo[3,2-b]pyrrole-1-carboxylic acid diethylamide [14];
(3aR,6aS)-4-(2S-Acetylamino-methyl-pentanoyl)-6-oxo-hexahydro-pyrrolo[3,2-b]pyrrole-1-carboxylic acid sec-butylamide [15];
(3aR,6aS)—N-{3-Methyl-1-[3-oxo-4(pyrrolidine-1-carbonyl)-hexahydro-pyrrolo[3,2-b]pyrrole-1-carbonyl]-butyl}-acetamide [16];
(3aR,6aS)-4-Benzoyl-6-oxo-hexahydro-pyrrolo[3,2-b]pyrrole-1-carboxylic acid 4,4-dimethyl-2-oxo-tetrahydro-furan-3R-yl ester [17];
(3aR,6aS)-4-Benzoyl-6-oxo-hexahydro-pyrrolo[3,2-b]pyrrole-1-carboxylic acid 4,4-dimethyl-2-oxo-tetrahydro-furan-3S-yl ester [18];
(3aS,6aR)-3-Oxo-hexahydro-furo[3,2-b]pyrrolecarboxylic acid 2,2-dimethyl-propyl ester [19];
(3aR,6aS)-1-Benzylcyclobutyl 4-benzoyl-6-oxohexahydropyrrolo[3,2-b]pyrrole-1(2H)-carboxylate;
(3aR,6aS)-1-Phenethylcyclobutyl 4-benzoyl-6-oxohexahydropyrrolo[3,2-b]pyrrole-1(2H)-carboxylate;
(3aR,6aS)-1-(Thiophen-3-yl)butan-2-yl 4-benzoyl-6-oxohexahydropyrrolo[3,2-b]pyrrole-1(2H)-carboxylate;
(3aR,6aS)-(1-(Phenoxymethyl)cyclobutyl)methyl 4-benzoyl-6-oxohexahydropyrrolo[3,2-b]pyrrole-1(2H)-carboxylate;
(3aR,6aS)-1-(Thiophen-2-yl)butan-2-yl 4-benzoyl-6-oxohexahydropyrrolo[3,2-b]pyrrole-1(2H)-carboxylate;
(3aR,6aS)-1-Isopropylcyclopropyl 4-benzoyl-6-oxohexahydropyrrolo[3,2-b]pyrrole-1(2H)-carboxylate;
(3aR,6aS)-5-Methyl-1-(thiophen-2-yl)hexan-3-yl 4-benzoyl-6-oxohexahydropyrrolo[3,2-b]pyrrole-1(2H)-carboxylate;
(3aR,6aS)-5,5-Dimethylhexan-3-yl 4-benzoyl-6-oxohexahydropyrrolo[3,2-b]pyrrole-1(2H)-carboxylate;
(3aR,6a)-3-Methyl-1-phenylbutyl 4-benzoyl-6-oxohexahydropyrrolo[3,2-b]pyrrole-1(2H)-carboxylate;
(3aS,6aR)-1-Benzylcyclobutyl 3-oxotetrahydro-2H-furo[3,2-b]pyrrole-4(5H)-carboxylate;
(3aS,6aR)-1-Phenethylcyclobutyl 3-oxotetrahydro-2H-furo[3,2-b]pyrrole-4(5H)-carboxylate;
(3aS,6aR)-1-(Thiophen-3-yl)butan-2-yl 3-oxotetrahydro-2H-furo[3,2-b]pyrrole-4(5H)-carboxylate;
(3aS,6aR)-(1-(Phenoxymethyl)cyclobutyl)methyl 3-oxotetrahydro-2H-furo[3,2-b]pyrrole-4(5H)-carboxylate;
(3aS,6aR)-1-(Thiophen-2-yl)butan-2-yl 3-oxotetrahydro-2H-furo[3,2-b]pyrrole-4(5H)-carboxylate;
(3aS,6aR)-1-Isopropylcyclopropyl 3-oxotetrahydro-2H-furo[3,2-b]pyrrole-4(5H)-carboxylate;
(3aS,6aR)-5-Methyl-1-(thiophen-2-yl)hexan-3-yl 3-oxotetrahydro-2H-furo[3,2-b]pyrrole-4(5H)carboxylate;
(3 aS,6aR)-3-Methyl-1-phenylbutyl 3-oxotetrahydro-2H-furo[3,2-b]pyrrole-4(5H)-carboxylate;
(3aS,6aR)-5,5-Dimethylhexan-3-yl 3-oxotetrahydro-2H-furo[3,2-b]pyrrole-4(5H)-carboxylate;
(3aS,6aR)-4-Ethylbiphenyl-3-yl 3-oxotetrahydro-2H-furo[3,2-b]pyrrole-4(5H)-carboxylate;
(3aR,6aS)-4-Benzoyl-6-oxo-N-(1-(thiophen-3-yl)butan-2-yl)hexahydropyrrolo[3,2-b]pyrrole-1(2H)-carboxamide;
(3aR,6aS)-6-Oxo-4(pyrrolidine-1-carbonyl)-N-(1-(thiophen-3-yl)butan-2-yl)hexahydropyrrolo[3,2-b]pyrrole-1(2H)-carboxamide;
(3aR,6aS)-4-Benzoyl-6-oxo-A-(1-(thiophen-2-yl)butan-2-yl)hexahydropyrrolo[3,2-b]pyrrole-1(2H)-carboxamide;
(3aR,6aS)-6-Oxo-pyrrolidine-1-carbonyl)-N-(1-(thiophen-2-yl)butan-2-yl)hexahydropyrrolo[3,2-b]pyrrole-1(2H)-carboxamide;
(3aR,6aS)-4-Benzoyl-6-oxo-N-((1-(phenoxymethyl)cyclobutyl)methyl)hexahydropyrrolo[3,2-b]pyrrole-1(2H)-carboxamide;
(3aR,6aS)-4-Benzoyl-N-(5-methyl-1-(thiophen-2-yl)hexan-3-yl)-6-oxohexahydro pyrrolo[3,2-b]pyrrole-1(2H)-carboxamide;
(3aR,6aS)-4-Benzoyl-N-(6-chloro-2-fluoro-3-methylbenzyl)-6-oxohexahydro pyrrolo[3,2-b]pyrrole-1(2H)-carboxamide;
(3aR,6aS)—N-(6-Chloro-2-fluoro-3-methylbenzyl)-6-oxo-4-(pyrrolidine-1-carbonyl)hexahydropyrrolo[3,2-b]pyrrole-1(2H)-carboxamide;
(3aR,6aS)-4-Benzoyl-N-(biphenyl-2-yl)-6-oxohexahydropyrrolo[3,2-b]pyrrole-1(2H)-carboxamide;
(3aR,6aS)-4-Benzoyl-N-(2-ethoxyphenyl)-6-oxohexahydropyrrolo[3,2-b]pyrrole-1(2H)-carboxamide;
(3aR,6aS)-4-Benzoyl-6-oxo N-(2-propylphenyl)hexahydropyrrolo[3,2-b]pyrrole-1(2H)-carboxamide;
(3aR,6aS)-4-Benzoyl-N-(2-chloro-5-(trifluoromethyl)phenyl)-6-oxohexahydropyrrolo[3,2-b]pyrrole-1(2H)-carboxamide;
(3aS,6aR)-3-Oxo-N-(1-(thiophen-3-yl)butan-2-yl)tetrahydro-2H-furo[3,2-b]pyrrole-4(5H)-carboxamide;
(3aS,6aR)-3-Oxo-N-(1-(thiophen-2-yl)butan-2-yl)tetrahydro-2H-furo[3,2-b]pyrrole-4(5H)-carboxamide;
(3aS,6aR)-3-Oxo-N-((1-(phenoxymethyl)cyclobutyl)methyl)tetrahydro-2H-furo[3,2-b]pyrrole-4(5H)-carboxamide;
(3aS,6aR)—N-(5-Methyl-1-(thiophen-2-yl)hexan-3-yl)-3-oxotetrahydro-2H-furo[3,2-b]pyrrole-4(5H)-carboxamide;
(3aS,6aR)—N-(6-Chloro-2-fluoro-3-methylbenzyl)-3-oxotetrahydro-2H-furo[3,2-b]pyrrole-4(5H)carboxamide;
(3aS,6aR)—N-(Biphenyl-2-yl)-3-oxotetrahydro-2H-furo[3,2-b]pyrrole-4(5H)-carboxamide;
(3aS,6aR)—N-(2-Ethoxyphenyl)-3-oxotetrahydro-2H-furo[3,2-b]pyrrole-4(5H)-carboxamide;
(3aS,6aR)-3-Oxo-N-(2-propylphenyl)tetrahydro-2H-furo[3,2-b]pyrrole-4(5H)-carboxamide;
(3aS,6aR)—N-(2-Chloro-5-(trifluoromethyl)phenyl)-3-oxotetrahydro-2H-furo[3,2-b]pyrrole-4(5H)-carboxamide;
(3aR,6aS)—S-6-Chloro-2-fluoro-3-methylbenzyl 4-benzoyl-6-oxohexahydropyrrolo[3,2-b]pyrrole-1(2H)-carbothioate;
(3aR,3aR,6aR)-6-chloro-2-fluoro-3-methylbenzyl 3-hydroxytetrahydro-2H-furo[3,2-b]pyrrole-4(5H)-carbothioate;
(3aR,6aS)-2-Ethoxy-4-methylphenyl 4-benzoyl-6-oxohexahydropyrrolo[3,2-b]pyrrole-1(2H)-carboxylate;
(3aR,6aS)-2-Isopropoxyphenyl 4-benzoyl-6-oxohexahydropyrrolo[3,2-b]pyrrole-1(2H)-carboxylate;
(3aR,6aS)-2-Propylphenyl 4-benzoyl-6-oxohexahydropyrrolo[3,2-b]pyrrole-1(2H)-carboxylate;
(3aR,6aS)-(2-Methyl-6-(trifluoromethyl)pyridin-3-yl)methyl 4-benzoyl-6-oxohexahydropyrrolo[3,2-b]pyrrole-1(2H)-carboxylate;
(3aR,6aS)-2-Fluoro-6-(trifluoromethyl)benzyl 4-benzoyl-6-oxohexahydropyrrolo[3,2-b]pyrrole-1(2H)-carboxylate;
(3aR,6aS)-6-Chloro-2-fluoro-3-methylbenzyl 4-benzoyl-6-oxohexahydropyrrolo[3,2-b]pyrrole-1(2)-carboxylate;
(3aS,6aR)-2-Propylphenyl 3-oxotetrahydro-2H-furo[3,2-b]pyrrole-4(5H)-carboxylate;
and pharmaceutically acceptable salts thereof.

42. A pharmaceutical or veterinary composition comprising a compound according to claim 41 and a pharmaceutically acceptable or veterinarily acceptable diluent, excipient and/or carrier.

43. A process for preparing a pharmaceutical or veterinary composition according to claim 42, said process comprising admixing a compound according to any one of claims 1 to 41 with a pharmaceutically acceptable or veterinarily acceptable diluent, excipient and/or carrier.

44. A compound according to claim 41 for use in medicine.

45. Use of a compound according to, claim 41 in the preparation of a medicament for treating a disease selected from osteoporosis, Paget's disease, Chagas's disease, malaria, gingival diseases, hypercalaemia, metabolic bone disease and diseases involving matrix or cartilage degradation.

46. Use according to claim 45 wherein the gingival disease is gingivitis or periodontitis.

47. Use according to claim 45 wherein the disease involving matrix or cartilage degradation is selected from osteoarthritis, rheumatoid arthritis and neoplastic diseases.

48. Use of a compound according to claim 41 for inhibiting a cysteine proteinase.

49. Use according to claim 48 wherein the cysteine proteinase is a CAC1 cysteine proteinase.

50. Use according to claim 49 wherein the CAC1 cysteine proteinase is selected from cathepsin K, cathepsin S, cathepsin F, cathepsin B, cathepsin L, cathepsin V, cathepsin C, falcipain and cruzipain.

51. A method of inhibiting a cysteine proteinase in a cell, said method comprising contacting said cell with a compound according to any one of claims 1 to 41.

52. A method of inhibiting a cysteine proteinase in a subject, said method comprising administering to the subject a pharmacologically effective amount of a compound according to claim 41.

53. A method of treating a disease selected from osteoporosis, Paget's disease, Chagas's disease, malaria, gingival diseases, hypercalaemia, metabolic bone disease and diseases involving matrix or cartilage degradation, in a subject, said method comprising administering to the subject a pharmacologically effective amount of a compound according to claim 41.

54. Use of a compound according to claim 41 in an assay for identifying further candidate compounds capable of inhibiting one or more cysteine proteinases.

55. Use according to claim 36 wherein said assay is a competitive binding assay.

56. Use according to claim 55 wherein said competitive binding assay comprises contacting a compound according to claim 41 with a cysteine proteinase and detecting any change in the interaction between the compound according to claim 41 and the cysteine proteinase.

57. A method of validating a known or putative cysteine proteinase as a therapeutic target, the method comprising:
(a) assessing the in vitro binding of a compound according to claim 41 to an isolated or known putative cysteine proteinase, providing a measure of potency; and optionally, one or more of the steps of:
(b) assessing the binding of a compound according to claim 41 to closely related homologous proteinases of the target and general housekeeping proteinases (e.g. trypsin) to provide a measure of selectivity;
(c) monitoring a cell-based functional marker of a particular cysteine proteinase activity in the presence of a compound according to claim 41 and
(d) monitoring an animal model-based functional marker of a particular cysteine proteinase activity in the presence of a compound according to claim 41.

58. Use of a compound according to claim 41 in the validation of a known or putative cysteine proteinase as a therapeutic target.

59. A process of preparing a compound of formula I as defined in claim 1, said process comprising the step of converting a compound of formula II to a compound of formula I, 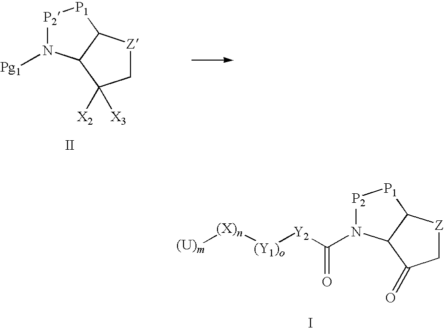
wherein
P₁ is 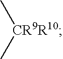
P₂′ is O, 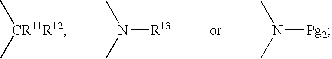
Z′ is O, 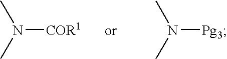
X₂ and X₃ together form ═O, or are each independently OR′, where R′ is H or alkyl;
Pg₁, Pg₂ and Pg₃ are each independently amine protecting groups; and
P₂, Z, Y₂, Y₁, X, U, R¹, R⁹⁻¹³, m, n and o are as defined in claim 1.

60. A process according to claim 59 wherein Pg₁, Pg₂ and Pg₃ are each independently selected from 9-fluorenylmethoxycarbonyl (Fmoc), tert-butoxycarbonyl (Boc), benzyloxycarbonyl (Cbz), allyloxycarbonyl (Alloc) and trichloroethoxycarbonyl (Treoc).

61. A process according to claim 60 which comprises the steps of:
(i) converting a compound of formula III to a compound of formula IV;
(ii) attaching said compound of formula IV to a solid phase resin via a linker to form an intermediate species of formula V;
(iii) removing protecting group Pg₁ from said intermediate species of formula V and converting to an intermediate species of formula VI; and
(iv) removing said compound of formula I from the solid phase resin

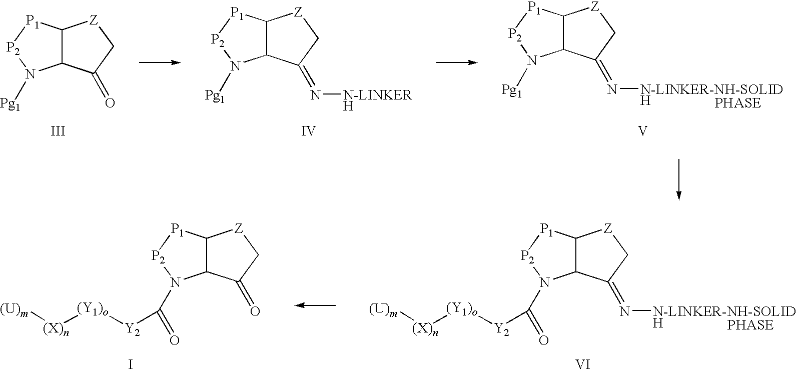

62. A process according to claim 61 wherein Pg₁ is Fmoc.

63. A process according to claim 61 which comprises attaching a compound of formula (15) to a solid phase resin to form an intermediate species of formula (16), and subsequently converting to a species of formula (17)

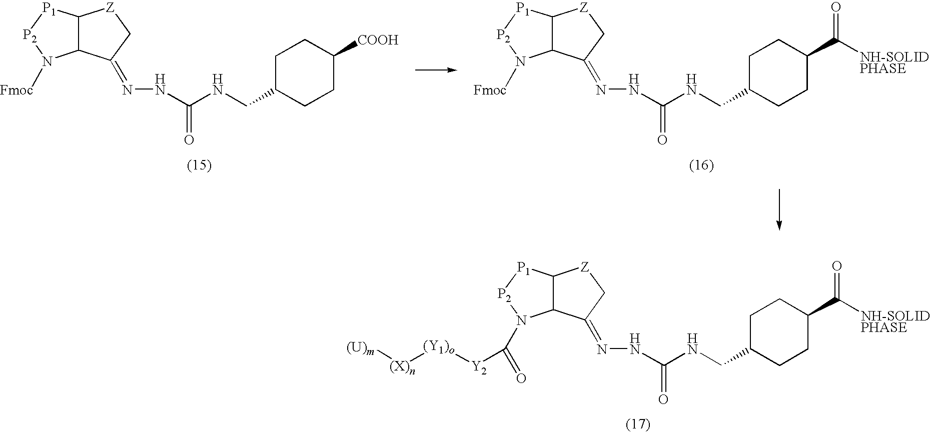

64. A process according to claim 61 which comprises removing protecting group Pg₁ and reacting the intermediate so produced with a compound selected from:
(U)ₘ(X)ₙ(Y₁)ₒ—O(CO)Cl;
(U)ₘ(X)ₙ(Y₁)ₒ—S(CO)Cl;
(U)ₘ(X)ₙ(Y₁)ₒ—N═C≡O; and
(U)ₘ(X)ₙ(Y₁)ₒ—NH(CO)Cl.

65. A process according to claim 64 wherein Z′ is 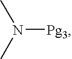
which further comprises the step of removing said Pg₃ group and reacting the compound so produced with a compound selected from:
R¹COOH;
R²SO₂Cl;
R¹N═C═O;
R¹OCOCl; and
R³COCl;
where R¹, R² and R³ are as defined in claim 1.

66. A process according to claim 61 which comprises the steps of: 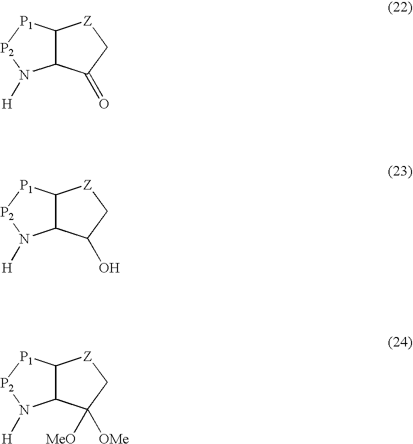
reacting a compound of formula (22), (23) or (24), where Z is O, 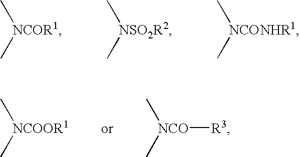
with a compound selected from:
(U)ₘ(X)ₙ(Y₁)ₒ—O(CO)Cl;
(U)ₘ(X)ₙ(Y₁)ₒ—S(CO)Cl;
(U)ₘ(X)ₙ(Y₁)ₒ—N═C≡O; and
(U)ₘ(X)ₙ(Y₁)ₒ—NH(CO)Cl.
where P₁, P₂, U, X, Y₁, m, n and o are as defined in claim 1.

67. A process according to claim 61 which comprises the steps of: 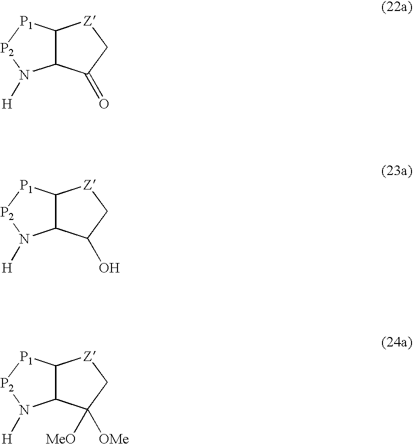
reacting a compound of formula (22a), (23a) or (24a), where Z′ is 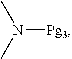
with a compound selected from:
(U)ₘ(X)ₙ(Y₁)ₒ—O(CO)Cl;
(U)ₘ(X)ₙ(Y₁)ₒ—S(CO)Cl;
(U)ₘ(X)ₙ(Y₁)ₒ—N═C≡O; and
(U)ₘ(X)ₙ(Y₁)ₒ—NH(CO)Cl.
where P₁, P₂, U, X, Y₁, m, n and o are as defined in claim 1;
converting said 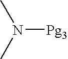
group to a group selected from

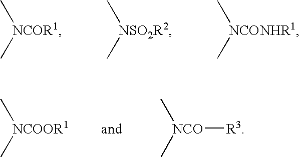

68. A compound, pharmaceutical composition, use or process substantially as described herein.
